SEQUENCE LISTING (104 sequences)

SEQ ID NO: 1 - protein, 716 aa, Influenza virus
MEDFVRQCFNPMIVELAEKTMKEYGEDLKIETNKFAAICTHLEVCFMYSDFHFINEQGESIIVELGDPNALLKHRFEIIEGRDRTMAWTVVNSICNTTGAEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLENFRAYVDGFEPNGYIEGKLSQMSKEVNARIEPFLKTTPRPLRLPNGPPCSQRSKFLLMDALKLSIEDPSHEGEGIPLYDAIKCMRTFFGWKEPNVVKPHEKGINPNYLLSWKQVLAELQDIENEEKIPKTKNMKKTSQLKWALGENMAPEKVDFDDCKDVGDLKQYDSDEPELRSLASWIQNEFNKACELTDSSWIELDEIGEDVAPIEHIASMRRNYFTSEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLIRSAIGQVSRPMFLYVRTNGTSKIKMKWGMEMRRCLLQSLQQIESMIEAESSVKEKDMTKEFFENKSETWPIGESPKGVEESSIGKVCRTLLAKSVFNSLYASPQLEGFSAESRKLLLIVQALRDNLEPGTFDLGGLYEAIEECLINDPWVLLNASWFNSFLTHALS

SEQ ID NO: 2 - protein, 757 aa, Influenza virus
MDVNPTLLFLKVPTQNAISTTFPYTGDPPYSHGTGTGYTMDTVNRTHQYSEKGRWTTNTETGAPQLNPIDGPLPEDNEPSGYAQTDCVLEAMAFLEESHPGIFENSCIETMEVVQQTRVDKLTQGRQTYDWTLNRNQPAATALANTIEVFRSNGLTANESGRLIDFLKDVMESMNKEEMGITTHFQRKRRVRDNMTKKMITQRTMGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYMTRNQPEWFRNVLSIAPIMFSNKMARLGKGYMFESKSMKLRTQIPAEMLASIDLKYFNDSTRKKIEKIRPLLIEGTASLSPGMMMGMFNMLSTVLGVSILNLGQKRYTKTTYWWDGLQSSDDFALIVNAPNHEGIQAGVDRFYRTCKLLGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFEIKKLWEQTRSKAGLLVSDGGPNLYNIRNLHIPEVCLKWELMDEDYQGRLCNPLNPFVSHKEIESMNNAVMMPAHGPAKNMEYDAVATTHSWIPKRNRSILNTSQRGVLEDEQMYQRCCNLFEKFFPSSSYRRPVGISSMVEAMVSRARIDARIDFESGRIKKEEFTEIMKICSTIEELRRQK

SEQ ID NO: 3 - protein, 759 aa, Influenza virus
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPITNTVHYPKIYKTYFERVERLKHGTFGPVHFRNQVKIRRRVDINPGHADLSAKEAQDVIMEVVFPNEVGARILTSESQLTITKEKKEELQDCKISPLMVAYMLERELVRKTRFLPVAGGTSSVYIEVLHLTQGTCWEQMYTPGGEVRNDDVDQSLIIAARNIVRRAAVSADPLASLLEMCHSTQIGGIRMVDILRQNPTEEQAVDICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDARVLFQNWGVEPIDNVMGMIGILPDMTPSIEMSMRGVRISKMGVDEYSSTERVVVSIDRFLRIRDQRGNVLLSPEEVSETQGTEKLTITYSSSMMWEINGPESVLVNTYQWIIRNWETVKIQWSQNPTMLYNKMEFEPFQSLVPKAIRGQYSGFVRTLFQQMRDVLGTFDTAQIIKLLPFAAAPPKQSRMQFSSFTVNVRGSGMRILVRGNSPVFNYNKATKRLTVLGKDAGTLTEDPDEGTAGVESAVLRGFLILGKEDKRYGPALSINELSNLAKGEKANVLIGQGDVVLVMKRKRDSSILTDSQTATKRIRMAIN

SEQ ID NO: 4 - protein, 498 aa, Influenza virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 5 - protein, 252 aa, Influenza virus
MSLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQMVTTTNPLIRHENRMVLASTTAKAMEQMAGSSEQAAEAMEVASQARQMVQAMRTIGTHPSSSAGLKNDLLENLQAYQKRMGVQMQRFK

SEQ ID NO: 6 - protein, 230 aa, Influenza virus
MDPNTVSSFQVDCFLWHVRKRVADQELGDAPFLDRLRRDQKSLRGRGSTLGLDIKTATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 7 - protein, 565 aa, Influenza virus
MKANLLVLLCALAAADADTICIGYHTNNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLEFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 8 - protein, 454 aa, Influenza virus
MNPNQKIITIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTQGALLNDKHSSGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTDKVMCVCRDNWHGSNRPWVSFDQNLDYQIGYICSGVFGDNPRPEDGTGSCGPVYVDGANGVKGFSYRYGNGVWIGRTKSHSSRHGFEMIWDPNGWTETDSKFSVRQDVVAMTDWSGYSGSFVQHPELTGLDCMRPCFWVELIRGRPKEKTIWTSASSISFCGVNSDTVDWSWPDGAELPFSIDK

SEQ ID NO: 9 - DNA, 2233 bp, Influenza virus
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagatttatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggggtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaaccttgaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact

SEQ ID NO: 10 - DNA, 2341 bp, Influenza virus
agcgaaagcaggcaaaccatttgaatggatgtcaatccgaccttacttttcttaaaagtgccaacacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaggcgatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaacaaagaagaaatggggatcacaactcattttcagagaaagagacgggtgagagacaatatgactaagaaaatgataacacagagaacaatgggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagcttcccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaggatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 11 - DNA, 2341 bp, Influenza virus
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaataacaaatacagttcattatccaaaaatctacaaaacttattttgaaagagtagaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggaaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcagcgattgaatcctatgcatcaacttttaagacattttcagaaggatgcgagagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccgttttttgagaatccgggaccaacgaggaaatgtactactgtctcccgaggaggtcagtgaaacacagggaacagagaaactgacaataacttactcatcgtcaatgatgtgggagattaatggtcctgaatcagtattggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 12 - DNA, 1565 bp, Influenza virus
agcaaaagcagggtagataatcactcactgagtgacatcaaaatcatggcgtctcaaggcaccaaacgatcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaataccttgaagaacatcccagtgcgggaaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcagaatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagggagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 13 - DNA, 1027 bp, Influenza virus
agcaaaagcaggtagatattgaaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 14 - DNA, 890 bp, Influenza virus
agcaaaagcagggtgacaaaaacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcagaatggaccaggcgatcatggataagaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 15 - DNA, 1775 bp, Influenza virus
agcaaaagcaggggaaaataaaaacaaccaaaatgaaggcaaacctactggtcctgttatgtgcacttgcagctgcagatgcagacacaatatgtataggctaccatacgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacattccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggaattccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgagattagaatttcagagatatgaggaaaaacacccttgtttctact

SEQ ID NO: 16 - DNA, 1413 bp, Influenza virus
agcaaaagcaggggtttaaaatgaatccaaatcagaaaataataaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaagtgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccgacaaagtgatgtgtgtgtgcagagacaattggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgtgaggcaagatgttgtggcaatgactgattggtcagggtatagcggaagtttcgttcaacatcctgagctgacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 17 - protein, 716 aa, Influenza virus
MEDFVRQCFNPMIVELAEKAMKEYGEDPKIETNKFAAICTHLEVCFMYSDFHFIDERGESIIVESGDPNALLKHRFEIIEGRDRIMAWTVVNSICNTTGVEKPKFLPDLYDYKENRFIEIGVTRREVHIYYLEKANKIKSEKTHIHIFSFTGEEMATKADYTLDEESRARIKTRLFTIRQEMASRSLWDSFRQSERGEETIEEKFEITGTMRKLADQSLPPNFPSLENFRAYVDGFEPNGCIEGKLSQMSKEVNAKIEPFLRTTPRPLRLPDGPLCHQRSKFLLMDALKLSIEDPSHEGEGIPLYDAIKCMKTFFGWKEPNIVKPHEKGINPNYLMAWKQVLAELQDIENEEKIPRTKNMKRTSQLKWALGENMAPEKVDFDDCKDVGDLKQYDSDEPEPRSLASWVQNEFNKACELTDSSWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRTAIGQVSRPMFLYVRTNGTSKIKMKWGMEMRRCLLQSLQQIESMIEAESSVKEKDMTKEFFENKSETWPIGESPRGVEEGSIGKVCRTLLAKSVFNSLYASPQLEGFSAESRKLLLIVQALRDNLEPGTFDLGGLYEAIEECLINDPWVLLNASWFNSFLTHALK

SEQ ID NO: 18 - protein, 757 aa, Influenza virus
MDVNPTLLFLKIPAQNAISTTFPYTGDPPYSHGTGTGYTMDTVNRTHQYSEKGKWTTNTETGAPQLNPIDGPLPEDNEPSGYAQTDCVLEAMAFLEESHPGIFENSCLETMEVVQQTRVDKLTQGRQTYDWTLNRNQPAATALANTIEVFRSNGLTANESGRLIDFLKDVMESMNKEEIEITTHFQRKRRVRDNMTKKMVTQRTIGKKKQRLNKRGYLIRALTLNTMTKDAERGKLKRRAIATPGMQIRGFVYFVETLARSICEKLEQSGLPVGGNEKKAKLANVVRKMMTNSQDTEISFTITGDNTKWNENQNPRMFLAMITYITRNQPEWFRNILSMAPIMFSNKMARLGKGYMFESKRMKIRTQIPAEMLASIDLKYFNESTKKKIEKIRPLLIDGTASLSPGMMMGMFNMLSTVLGVSILNLGQKKYTKTIYWWDGLQSSDDFALIVNAPNHEGIQAGVDRFYRTCKLVGINMSKKKSYINKTGTFEFTSFFYRYGFVANFSMELPSFGVSGVNESADMSIGVTVIKNNMINNDLGPATAQMALQLFIKDYRYTYRCHRGDTQIQTRRSFELKKLWDQTQSKVGLLVSDGGPNLYNIRNLHIPEVCLKWELMDDDYRGRLCNPLNPFVSHKEIDSVNNAVVMPAHGPAKSMEYDAVATTHSWIPKRNRSILNTSQRGILEDEQMYQKCCNLFEKFFPSSSYRRPVGISSMVEAMVSRARIDARVDFESGRIKKEEFSEIMKICSTIEELRRQK

SEQ ID NO: 19 - protein, 759 aa, Influenza virus
MERIKELRDLMSQSRTREILTKTTVDHMAIIKKYTSGRQEKNPALRMKWMMAMRYPITADKRIMDMIPERNEQGQTLWSKTNDAGSDRVMVSPLAVTWWNRNGPTTSTVHYPKVYKTYFEKVERLKHGTFGPVHFRNQVKIRRRVDTNPGHADLSAKEAQDVIMEVVFPNEVGARILTSESQLAITKEKKEELQDCKIAPLMVAYMLERELVRKTRFLPVAGGTGSVYIEVLHLTQGTCWEQMYTPGGEVRNDDVDQSLIIAARNIVRRAAVSADPLASLLEMCHSTQIGGVRMVDILRQNPTEEQAVDICKAAIGLRISSSFSFGGFTFKRTSGSSVKKEEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGIESIDNVMGMIGILPDMTPSTEMSLRGIRVSKMGVDEYSSTERVVVSIDRFLRVRDQRGNVLLSPEEVSETQGTEKLTITYSSSMMWEINGPESVLVNTYQWIIRNWEIVKIQWSQDPTMLYNKMEFEPFQSLVPKATRSRYSGFVRTLFQQMRDVLGTFDTVQIIKLLPFAAAPPEQSRMQFSSLTVNVRGSGLRILVRGNSPVFNYNKATKRLTVLGKDAGALTEDPDEGTSGVESAVLRGFLILGKEDKRYGPALSINELSNLAKGEKANVLIGQGDVVLVMKRKRDSSILTDSQTATKRIRMAIN

SEQ ID NO: 20 - protein, 319 aa, Influenza virus
MASQGTKRSYEQMETGGERQDATEIRASVGRMIGGIGRFYIQMCTELKLSDYDGRLIQNSITIERMVLSAFDERRNKYLEEHPSAGKDPKKTGGPIYRRVDGKWMRELILYDKEEIRRVWRQANNGEDATAGLTHIMIWHSNLNDATYQRTRALVRTGMDPRMCSLMQGSTLPRRSGAAGAAVKGVGTIAMELIRMIKRGINDRNFWRGENGRRTRVAYERMCNILKGKFQTAAQRAMMDQVRESRNPGNAEIEDLIFLARSALILRGSVAHKSCLPACVYGLAVASGHDFEREGYSLVGIDPFKLLQNSQVVSLMRPN

SEQ ID NO: 21 - protein, 252 aa, Influenza virus
MSLLTEVETYVLSIIPSGPLKAEIAQRLESVFAGKNTDLEALMEWLKTRPILSPLTKGILGFVFTLTVPSERGLQRRRFVQNALNGNGDPNNMDRAVKLYKKLKREITFHGAKEVSLSYSTGALASCMGLIYNRMGTVTTEAAFGLVCATCEQIADSQHRSHRQMATTTNPLIRHENRMVLASTTAKAMEQMAGSSEQAAEAMEVANQTRQMVHAMRTIGTHPSSSAGLKDDLLENLQAYQKRMGVQMQRFK

SEQ ID NO: 22 - protein, 219 aa, Influenza virus
MDSNTMSSFQVDCFLWHIRKRFADNGLGDAPFLDRLRRDQKSLKGRGNTLGLDIETATLVGKQIVEWILKEESSETLRMTIASVPTSRYLSDMTLEEMSRDWFMLMPRQKIIGPLCVRLDQAIMEKNIVLKANFSVIFNRLETLILLRAFTEEGAIVGEISPLPSLPGHTYEDVKNAVGVLIGGLEWNGNTVRVSENIQRFAWRNCDENGRPSLPPEQK

SEQ ID NO: 23 - DNA, 2151 bp, Influenza virus
atggaagactttgtgcgacaatgcttcaatccaatgatcgtcgagcttgcggaaaaggcaatgaaagaatatggggaagatccgaaaatcgaaactaacaagtttgctgcaatatgcacacatttggaagtttgtttcatgtattcggatttccatttcatcgacgaacggggtgaatcaataattgtagaatctggtgacccgaatgcactattgaagcaccgatttgagataattgaaggaagagaccgaatcatggcctggacagtggtgaacagtatatgtaacacaacaggggtagagaagcctaaatttcttcctgatttgtatgattacaaagagaaccggttcattgaaattggagtaacacggagggaagtccacatatattacctagagaaagccaacaaaataaaatctgagaagacacacattcacatcttttcattcactggagaggagatggccaccaaagcggactacacccttgacgaagagagcagggcaagaatcaaaactaggcttttcactataagacaagaaatggccagtaggagtctatgggattcctttcgtcagtccgaaagaggcgaagagacaattgaagaaaaatttgagattacaggaactatgcgcaagcttgccgaccaaagtctcccaccgaacttccccagccttgaaaactttagagcctatgtagatggattcgagccgaacggctgcattgagggcaagctttcccaaatgtcaaaagaagtgaacgccaaaattgaaccattcttgaggacgacaccacgccccctcagattgcctgatgggcctctttgccatcagcggtcaaagttcctgctgatggatgctctgaaattaagtattgaagacccgagtcacgagggggagggaataccactatatgatgcaatcaaatgcatgaagacattctttggctggaaagagcctaacatagtcaaaccacatgagaaaggcataaatcccaattacctcatggcttggaagcaggtgctagcagagctacaggacattgaaaatgaagagaagatcccaaggacaaagaacatgaagagaacaagccaattgaagtgggcactcggtgaaaatatggcaccagaaaaagtagactttgatgactgcaaagatgttggagaccttaaacagtatgacagtgatgagccagagcccagatctctagcaagctgggtccaaaatgaattcaataaggcatgtgaattgactgattcaagctggatagaacttgatgaaataggagaagatgttgccccgattgaacatatcgcaagcatgaggaggaactattttacagcagaagtgtcccactgcagggctactgaatacataatgaagggagtgtacataaatacggccttgctcaatgcatcctgtgcagccatggatgactttcagctgatcccaatgataagcaaatgtaggaccaaagaaggaagacggaaaacaaacctgtatgggttcattataaaaggaaggtctcatttgagaaatgatactgatgtggtgaactttgtaagtatggagttctcactcactgacccgagactggagccacacaaatgggaaaaatactgtgttcttgaaataggagacatgctcttgaggactgcgataggccaagtgtcgaggcccatgttcctatatgtgagaaccaatggaacctccaagatcaagatgaaatggggcatggaaatgaggcgctgccttcttcagtctcttcagcagattgagagcatgattgaggccgagtcttctgtcaaagagaaagacatgaccaaggaattctttgaaaacaaatcggaaacatggccaatcggagagtcacccaggggagtggaggaaggctctattgggaaagtgtgcaggaccttactggcaaaatctgtattcaacagtctatatgcgtctccacaacttgaggggttttcggctgaatctagaaaattgcttctcattgttcaggcacttagggacaacctggaacctggaaccttcgatcttggggggctatatgaagcaatcgaggagtgcctgattaatgatccctgggttttgcttaatgcatcttggttcaactccttcctcacacatgcactgaagtag

SEQ ID NO: 24 - DNA, 2341 bp, Influenza virus
agcgaaagcaggcaaaccatttgaatggatgtcaatccgactctacttttcctaaaaattccagcgcaaaatgccataagcaccacattcccttatactggagatcctccatacagccatggaacaggaacaggatacaccatggacacagtaaacagaacacaccaatactcagaaaagggaaagtggacgacaaacacagagactggtgcaccccagctcaacccgattgatggaccactacctgaggataatgaaccaagtgggtatgcacaaacagactgtgttctagaggctatggctttccttgaagaatcccacccaggaatatttgagaattcatgccttgaaacaatggaagttgttcaacaaacaagggtagataaactaactcaaggtcgccagacttatgattggacattaaacagaaatcaaccggcagcaactgcattggccaacaccatagaagtctttagatcgaatggcctaacagctaatgagtcaggaaggctaatagatttcttaaaggatgtaatggaatcaatgaacaaagaggaaatagagataacaacccactttcaaagaaaaaggagagtaagagacaacatgaccaagaagatggtcacgcaaagaacaatagggaagaaaaaacaaagactgaataagagaggctatctaataagagcactgacattaaatacgatgaccaaagatgcagagagaggcaagttaaaaagaagggctatcgcaacacctgggatgcagattagaggtttcgtatactttgttgaaactttagctaggagcatttgcgaaaagcttgaacagtctgggctcccagtagggggcaatgaaaagaaggccaaactggcaaatgttgtgagaaagatgatgactaattcacaagacacagagatttctttcacaatcactggggacaacactaagtggaatgaaaatcaaaatcctcgaatgttcctggcgatgattacatatatcaccagaaatcaacccgagtggttcagaaacatcctgagcatggcacccataatgttctcaaacaaaatggcaagactagggaaagggtacatgttcgagagtaaaagaatgaagattcgaacacaaataccagcagaaatgctagcaagcattgacctgaagtacttcaatgaatcaacaaagaagaaaattgagaaaataaggcctcttctaatagatggcacagcatcactgagtcctgggatgatgatgggcatgttcaacatgctaagtacggtcttgggagtctcgatactgaatcttggacaaaagaaatacaccaagacaatatactggtgggatgggctccaatcatccgacgattttgctctcatagtgaatgcaccaaaccatgagggaatacaagcaggagtggacagattctacaggacctgcaagttagtgggaatcaacatgagcaaaaagaagtcctatataaataagacagggacatttgaattcacaagctttttttatcgctatggatttgtggctaattttagcatggagctacccagctttggagtgtctggagtaaatgaatcagctgacatgagtattggagtaacagtgataaagaacaacatgataaacaatgaccttggacctgcaacggcccagatggctcttcaattgttcatcaaagactacagatacacatataggtgccataggggagacacacaaattcagacaagaagatcatttgagttaaagaagctgtgggatcaaacccaatcaaaggtagggctattagtatcagatggaggaccaaacttatacaatatacggaatcttcacattcctgaagtctgcttaaaatgggagctaatggatgatgattatcggggaagactttgtaatcccctgaatccctttgtcagtcataaagagattgattctgtaaacaatgctgtggtaatgccagcccatggtccagccaaaagcatggaatatgatgccgttgcaactacacattcctggattcccaagaggaatcgttctattctcaacacaagccaaaggggaattcttgaggatgaacagatgtaccagaagtgctgcaatctattcgagaaatttttccctagcagttcatataggagaccggttggaatttctagcatggtggaggccatggtgtctagggcccggattgatgccagggtcgacttcgagtctggacggatcaagaaagaagagttctctgagatcatgaagatctgttccaccattgaagaactcagacggcaaaaataatgaatttaacttgtccttcatgaaaaaatgccttgtttctact

SEQ ID NO: 25 - DNA, 2280 bp, Influenza virus
atggagagaataaaagaactgagagatctaatgtcgcagtcccgcactcgcgagatactcactaagaccactgtggaccatatggccataatcaaaaagtacacatcaggaaggcaagagaagaaccccgcactcagaatgaagtggatgatggcaatgagatacccaattacagcagacaagagaataatggacatgattccagagaggaatgaacaaggacaaaccctctggagcaaaacaaacgatgctggatcagaccgagtgatggtatcacctctggccgtaacatggtggaataggaatggcccaacaacaagtacagttcattaccctaaggtatataaaacttatttcgaaaaggtcgaaaggttgaaacatggtaccttcggccctgtccacttcagaaatcaagttaaaataaggaggagagttgatacaaaccctggccatgcagatctcagtgccaaggaggcacaggatgtgattatggaagttgttttcccaaatgaagtgggggcaagaatactgacatcagagtcacagctggcaataacaaaagagaagaaagaagagctccaggattgtaaaattgctcccttgatggtggcgtacatgctagaaagagaattggtccgtaaaacaaggtttctcccagtagccggcggaacaggcagtgtttatattgaagtgttgcacttaacccaagggacgtgctgggagcagatgtacactccaggaggagaagtgagaaatgatgatgttgaccaaagtttgattatcgctgctagaaacatagtaagaagagcagcagtgtcagcagacccattagcatctctcttggaaatgtgccacagcacacagattggaggagtaaggatggtggacatccttagacagaatccaactgaggaacaagccgtagacatatgcaaggcagcaatagggttgaggattagctcatctttcagttttggtgggttcactttcaaaaggacaagcggatcatcagtcaagaaagaagaagaagtgctaacgggcaacctccaaacactgaaaataagagtacatgaagggtatgaagaattcacaatggttgggagaagagcaacagctattctcagaaaggcaaccaggagattgatccagttgatagtaagcgggagagacgagcagtcaattgctgaggcaataattgtggccatggtattctcacaggaggattgcatgatcaaggcagttaggggcgatctgaactttgtcaatagggcaaaccagcgactgaaccccatgcaccaactcttgaggcatttccaaaaagatgcaaaagtgcttttccagaactggggaattgaatccatcgacaatgtgatgggaatgatcggaatactgcccgacatgaccccaagcacggagatgtcgctgagagggataagagtcagcaaaatgggagtagatgaatactccagcacggagagagtggtagtgagtattgaccgatttttaagggttagagatcaaagagggaacgtactattgtctcccgaagaagtcagtgaaacgcaaggaactgagaagttgacaataacttattcgtcatcaatgatgtgggagatcaatggccctgagtcagtgctagtcaacacttatcaatggataatcaggaactgggaaattgtgaaaattcaatggtcacaagatcccacaatgttatacaacaaaatggaatttgaaccatttcagtctcttgtccctaaggcaaccagaagccggtacagtggattcgtaaggacactgttccagcaaatgcgggatgtgcttgggacatttgacactgtccaaataataaaacttctcccctttgctgctgccccaccagaacagagtaggatgcaattttcctcattgactgtgaatgtgagaggatcagggttgaggatactggtaagaggcaattctccagtattcaattacaacaaggcaaccaaacgacttacagttcttggaaaggatgcaggtgcattgactgaagatccagatgaaggcacatctggggtggagtctgctgtcctgagaggatttctcattttgggcaaagaagacaagagatatggcccagcattaagcatcaatgaactgagcaatcttgcaaaaggagagaaggctaatgtgctaattgggcaaggggacgtagtgttggtaatgaaacgaaaacgggactctagcatacttactgacagccagacagcgaccaaaagaattcggatggccatcaattag

SEQ ID NO: 26 - DNA, 958 bp, Influenza virus
atggcgtctcaaggcaccaaacgatcatatgaacaaatggagactggtggggagcgccaggatgccacagaaatcagagcatctgtcggaagaatgattggtggaatcgggagattctacatccaaatgtgcactgaactcaaactcagtgattatgatggacgactaatccagaatagcataacaatagagaggatggtgctttctgcttttgatgagagaagaaataaatacctagaagagcatcccagtgctgggaaggaccctaagaaaacaggaggacccatatatagaagagtagacggaaagtggatgagagaactcatcctttatgacaaagaagaaataaggagagtttggcgccaagcaaacaatggcgaagatgcaacagcaggtcttactcatatcatgatttggcattccaacctgaatgatgccacatatcagagaacaagagcgcttgttcgcaccggaatggatcccagaatgtgctctctaatgcaaggttcaacacttcccagaaggtctggtgccgcaggtgctgcggtgaaaggagttggaacaatagcaatggagttaatcagaatgatcaaacgtggaatcaatgaccgaaatttctggaggggtgaaaatggacgaaggacaagggttgcttatgaaagaatgtgcaatatcctcaaaggaaaatttcaaacagctgcccagagggcaatgatggatcaagtaagagaaagtcgaaacccaggaaacgctgagattgaagacctcattttcctggcacggtcagcactcattctgaggggatcagttgcacataaatcctgcctgcctgcttgtgtgtatgggcttgcagtagcaagtgggcatgactttgaaagggaagggtactcactggtcgggatagacccattcaaattactccaaaacagccaagtggtcagcctgatgagaccaaatg

SEQ ID NO: 27 - DNA, 982 bp, Influenza virus
atgagtcttctaaccgaggtcgaaacgtacgttctttctatcatcccgtcaggccccctcaaagccgagatcgcgcagagactggaaagtgtctttgcaggaaagaacacagatcttgaggctctcatggaatggctaaagacaagaccaatcttgtcacctctgactaagggaattttaggatttgtgttcacgctcaccgtgcccagtgagcgaggactgcagcgtagacgctttgtccaaaatgccctaaatgggaatggggacccgaacaacatggatagagcagttaaactatacaagaagctcaaaagagaaataacgttccatggggccaaggaggtgtcactaagctattcaactggtgcacttgccagttgcatgggcctcatatacaacaggatgggaacagtgaccacagaagctgcttttggtctagtgtgtgccacttgtgaacagattgctgattcacagcatcggtctcacagacagatggctactaccaccaatccactaatcaggcatgaaaacagaatggtgctggctagcactacggcaaaggctatggaacagatggctggatcgagtgaacaggcagcggaggccatggaggttgctaatcagactaggcagatggtacatgcaatgagaactattgggactcatcctagctccagtgctggtctgaaagatgaccttcttgaaaatttgcaggcctaccagaagcgaatgggagtgcagatgcagcgattcaagtgatcctctcgtcattgcagcaaatatcattgggatcttgcacctgatattgtggattactgatcgtctttttttcaaatgtatttatcgtcgctttaaatacggtttgaaaagagggccttctacggaaggagtgcctgagtccatgagggaagaatatcaacaggaacagcagagtgctgtggatgttgacgatggtcattttgtcaacatagagctagagtaa

SEQ ID NO: 28 - DNA, 865 bp, Influenza virus
atggactccaacaccatgtcaagctttcaggtagactgtttcctttggcatatccgcaagcgatttgcagacaatggattgggtgatgccccattccttgatcggctccgccgagatcaaaagtccttaaaaggaagaggcaacacccttggcctcgatatcgaaacagccactcttgttgggaaacaaatcgtggaatggatcttgaaagaggaatccagcgagacacttagaatgacaattgcatctgtacctacttcgcgctacctttctgacatgaccctcgaggaaatgtcacgagactggttcatgctcatgcctaggcaaaagataataggccctctttgcgtgcgattggaccaggcgatcatggaaaagaacatagtactgaaagcgaacttcagtgtaatctttaaccgattagagaccttgatactactaagggctttcactgaggagggagcaatagttggagaaatttcaccattaccttctcttccaggacatacttatgaggatgtcaaaaatgcagttggggtcctcatcggaggacttgaatggaatggtaacacggttcgagtctctgaaaatatacagagattcgcttggagaaactgtgatgagaatgggagaccttcactacctccagagcagaaatgaaaagtggcgagagcaattgggacagaaatttgaggaaataaggtggttaattgaagaaatgcggcacagattgaaagcgacagagaatagtttcgaacaaataacatttatgcaagccttacaactactgcttgaagtagaacaagagataagagctttctcgtttcagcttatttaatgataaaaaacacccttgtttctactg

SEQ ID NO: 29 - protein, 758 aa, Influenza virus
MDVNPTLLFLKIPAQNAISTTFPYTGDPPYSHGTGTGYTMDTVNRTHQYSEKGKWTTNTETGAPQLNPIDGPLPEDNEPSGYAQTDCVLEAMAFLEESHPGIFENSCLETMEVVQQTRVDRLTQGRQTYDWTLNRNQPAATALANTIEVFRSNGLTANESGRLIDFLKDVMESMDKEEIEITTHFQRKRRVRDNMTKKMVTQRTIGKKKQRVNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYITKNQPEWFRNILSIAPIMFSNKMARLGKGYMFESKRMKLRTQIPAEMLASIDLKYFNESTRKKIEKIRPLLIDGTASLSPGMMMGMFNMLSTVLGVSILNLGQKKYTKTTYWWDGLQSSDDFALIVNAPNHEGIQAGVDRFYRTCKLVGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFELKKLWEQTRSKAGLLVSDGGPNLYNIRNLHIPEVCLKWELMDEDYQGRLCNPLNPFVSHKEIESVNNAVVMPAHGPAKSMEYDAVATTHSWIPKRNRSILNTSQRGILEDEQMYQKCCNLFEKFFPSSSYRRPVGISSMVEAMVSRARIDARIDFESGRIKKEEFSEIMKICSTIEELRRQKQ

SEQ ID NO: 30 - protein, 716 aa, Influenza virus; identical to SEQ ID NO: 1

SEQ ID NO: 31 - protein, 326 aa, Influenza virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRDPGNAEFEDLTFLARSALILRGSVAHKSCLPACVYGPAVASGYDFEREGYSLVGIDPFRLLQNSQVYSLIRPNENPAHKS

SEQ ID NO: 32 - protein, 252 aa, Influenza virus; identical to SEQ ID NO: 5

SEQ ID NO: 33 - DNA, 2299 bp, Influenza virus
aatatggaaagaataaaagagctaaggaatctgatgtcacaatctcgcactcgcgagatacttacaaaaactactgtagaccacatggccataatcaagaaatacacatcaggaagacaggagaaaaacccatcacttagaatgaaatggatgatggcaatgaaatacccaattacagcagataaaaggataacggaaatgattcctgaaagaaatgagcaaggacagacattatggagtaaagtgaatgatgccggatcagaccgagtgatgatatcacccctggctgtgacatggtggaacagaaatggaccagtggcaagtactattcactatccaaaaatctacaaaacttactttgaaaaggttgaaaggttaaaacatggaacctttggccctgtacactttagaaaccaagtcaaaatacgccgaagagtcgacataaatcctggtcatgcagacctcagcgccaaggaggcacaggatgtaattatggaagttgttttccctaatgaagtgggagccagaatactaacatcagaatcgcaattaacgataaccaaggagaaaaaagaagaactccagaattgcaaaatttcccctttgatggttgcatacatgttagagagggaacttgtccgcaaaacgagatttctcccggttgctggtggaacaagcagtgtgtacattgaagttttgcatttaacacaggggacatgctgggagcagatgtacactccaggtggggaggtgaggaatgatgatgttgatcaaagcctaattattgctgctaggaacatagtgagaagagctgcagtatcagcagatccactagcatctttattagaaatgtgccatagcacacagattggtgggacaaggatggtggatattctcaggcaaaatccaacagaagaacaagctgtggatatatgcaaagcagcaatggggctgagaatcagttcatccttcagttttggcggattcacatttaagagaacaagtggatcatcagtcaaaagggaggaagaagtgctcacgggcaatctgcaaacattgaagctaactgtgcatgagggatatgaagagttcacaatggttgggaaaagggcaacagctatactcagaaaagcaaccaggagattgattcaactaatagtgagtggaagagacgaacagtcaatagtcgaagcaatagttgtagcaatggtattctcacaagaagattgcatggtaaaagcagttagaggtgatctgaatttcgttaatagagcgaatcagcggttgaatcccatgcatcaacttttgagacattttcagaaggatgctaaagtacttttcttaaattggggaattgaacctatcgacaatgtgatgggaatgattgggatattacctgatatgactccaagtaccgagatgtcaatgagaggagtgagagtcagcaaaatgggtgtagatgaatactccaatgctgaaagggtagtggtgagcattgaccgttttttgagagtccgggaccaaagaggaaatgtactactgtctccagaggaagtcagtgaaacacagggaacagagaaactgacaataacttactcttcatcaatgatgtgggagattaatggccctgagtcagtgttgatcaatacctatcagtggatcatcagaaactgggagactgttaaaattcagtggtctcagaaccctacaatgctatacaataaaatggaattcgagccatttcagtctctagtccctaaggccattagaggccaatacagtgggtttgttagaactctatttcaacaaatgagggatgtgcttgggacctttgacacaactcagataataaaacttcttccctttgcagccgctccaccaaagcaaagtagaatgcaattctcatcattgactgtgaatgtgaggggatcaggaatgagaatacttgtaaggggtaattctccagtattcaactacaacaagaccactaagagactcacagtcctcggaaaggatgctggcactttaactgaagacccagatgaaggcacagctggagtggaatctgctgttctaaggggattcctcattctaggcaaagaagatagaagatatgggccagcattaagcatcaatgaattgagcaaccttgcgaaaggggaaaaagctaatgtgctaattgggcaaggggacgtagtgttggtaatgaaacgaaaacgggactctagcatacttactgacagccagacagcgaccaaaagaattcggatggccatcaattaatttcgaataatttaaa

SEQ ID NO: 34 - DNA, 2277 bp, Influenza virus
atggaacgcattaaagaactgcgcaacctgatgagccagagccgcacccgcgaaattctgaccaaaaccaccgtggatcatatggcgattattaaaaaatataccagcggccgccaggaaaaaaacccgagcctgcgcatgaaatggatgatggcgatgaaatatccgattaccgcggataaacgcattaccgaaatgattccggaacgcaacgaacagggccagaccctgtggagcaaagtgaacgatgcgggcagcgatcgcgtgatgattagcccgctggcggtgacctggtggaaccgcaacggcccggtggcgagcaccattcattatccgaaaatttataaaacctattttgaaaaagtggaacgcctgaaacatggcacctttggcccggtgcattttcgcaaccaggtgaaaattcgccgccgcgtggatattaacccgggccatgcggatctgagcgcgaaagaagcgcaggatgtgattatggaagtggtgtttccgaacgaagtgggcgcgcgcattctgaccagcgaaagccagctgaccattaccaaagaaaaaaaagaagaactgcagaactgcaaaattagcccgctgatggtggcgtatatgctggaacgcgaactggtgcgcaaaacccgctttctgccggtggcgggcggcaccagcagcgtgtatattgaagtgctgcatctgacccagggcacctgctgggaacagatgtataccccgggcggcgaagtgcgcaacgatgatgtggatcagagcctgattattgcggcgcgcaacattgtgcgccgcgcggcggtgagcgcggatccgctggcgagcctgctggaaatgtgccatagcacccagattggcggcacccgcatggtggatattctgcgccagaacccgaccgaagaacaggcggtggatatttgcaaagcggcgatgggcctgcgcattagcagcagctttagctttggcggctttacctttaaacgcaccagcggcagcagcgtgaaacgcgaagaagaagtgctgaccggcaacctgcagaccctgaaactgaccgtgcatgaaggctatgaagaatttaccatggtgggcaaacgcgcgaccgcgattctgcgcaaagcgacccgccgcctgattcagctgattgtgagcggccgcgatgaacagagcattgtggaagcgattgtggtggcgatggtgtttagccaggaagattgcatggtgaaagcggtgcgcggcgatctgaactttgtgaaccgcgcgaaccagcgcctgaacccgatgcatcagctgctgcgccattttcagaaagatgcgaaagtgctgtttctgaactggggcattgaaccgattgataacgtgatgggcatgattggcattctgccggatatgaccccgagcaccgaaatgagcatgcgcggcgtgcgcgtgagcaaaatgggcgtggatgaatatagcaacgcggaacgcgtggtggtgagcattgatcgctttctgcgcgtgcgcgatcagcgcggcaacgtgctgctgagcccggaagaagtgagcgaaacccagggcaccgaaaaactgaccattacctatagcagcagcatgatgtgggaaattaacggcccggaaagcgtgctgattaacacctatcagtggattattcgcaactgggaaaccgtgaaaattcagtggagccagaacccgaccatgctgtataacaaaatggaatttgaaccgtttcagagcctggtgccgaaagcgattcgcggccagtatagcggctttgtgcgcaccctgtttcagcagatgcgcgatgtgctgggcacctttgataccacccagattattaaactgctgccgtttgcggcggcgccgccgaaacagagccgcatgcagtttagcagcctgaccgtgaacgtgcgcggcagcggcatgcgcattctggtgcgcggcaacagcccggtgtttaactataacaaaaccaccaaacgcctgaccgtgctgggcaaagatgcgggcaccctgaccgaagatccggatgaaggcaccgcgggcgtggaaagcgcggtgctgcgcggctttctgattctgggcaaagaagatcgccgctatggcccggcgctgagcattaacgaactgagcaacctggcgaaaggcgaaaaagcgaacgtgctgattggccagggcgatgtggtgctggtgatgaaacgcaaacgcgatagcagcattctgaccgatagccagaccgcgaccaaacgcattcgcatggcgattaac

SEQ ID NO: 35 - DNA, 2201 bp, Influenza virus
gattcgaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtatgcttcatgtattcagattttcatttcatcaatgagcaaggcgaatcaataatagtagagcctgaggacccaaatgcacttttaaagcacagatttgagataatagagggacgagatcgtacaatggcatggacagttgtaaacagtatttgcaacaccacaggagctgagaaaccaaagtttctgccagatctgtatgattacaaagagaatagattcatcgagattggagtgacaaggagggaagttcacatatactatctggaaaaggccaacaaaattaaatctgagaagacacacattcacattttctcattcactggcgaagaaatggccacaaaggccgattacactctcgatgaagaaagcagggctaggattaaaaccagactattcaccataagacaagaaatggcaagcagaggtctttgggactcctttcgtcagtccgaaagaggcgaagaaacaattgaagaaagatttgaaatcacagggacaatgcgcaggctcgctgaccaaagccttccgccgaacttctcctgcattgagaattttagagcctatgtggatggatttgaaccgaacggctacattgagggcaagctttctcaaatgtccaaagaagtaaatgctagaattgagccttttttgaaaacaacaccacgaccaattagacttccggatgggcctccttgttttcagcggtcaaaattcctgctgatggattctttaaaattaagcattgaggatccaaatcatgaaggagagggaataccactatatgatgcaatcaagtgtatgagaacattctttggatggaaagaaccctctgttgtcaagccacacgggaagggaataaatccgaattatctgctgtcatggaagcaggtattggaagagctgcaggacattgagagtgaggagaagattccaagaacaaaaaacatgaaaaaaacgagtcagctaaagtgggcacttggtgagaacatggcaccagagaaggtggattttgatgactgtaaagatataagcgatttgaagcaatatgatagtgacgaacctgaattaaggtcattttcaagttggatccagaatgagttcaacaaggcatgcgagctgaccgattcaatctggatagagctcgatgagattggagaagatgtggccccgattgaacacattgcaagcatgagaagaaattacttcacagctgaggtgtcccattgcagagccacagaatatataatgaagggggtatacattaatactgctttgcttaatgcatcctgtgcagcaatggatgatttccaactaattcccatgataagcaaatgtagaactaaagagggaaggagaaagaccaatttgtacggcttcatcgtaaaaggaagatctcacttaaggaatgacaccgatgtggtaaactttgtgagcatggagttttccctcactgacccaagacttgagccacacaaatgggagaagtactgtgttcttgagataggagatatgcttctaaggagtgcaataggccaagtgtcaaggcccatgttcttgtatgtaaggacaaatggaacctcaaaaattaaaatgaaatggggaatggagatgaggcgttgcctcctccaatcccttcaacaaatagagagcatgattgaagctgagtcctccgtcaaggagaaagacatgacaaaagagttttttgagaatagatcagaaacatggcccattggagagtcaccaaaaggagtggaagaaggttccattgggaaagtatgcaggacactattggctaagtcagtattcaatagtctgtatgcatctccacaattagaaggattttcagctgagtcaagaaagttgctcctcattgttcaggctcttagggacaatctggaacctgggacctttgatcttggggggctatatgaagcaattgaggagtgcctgattaatgatccctgggttttgcttaatgcttcttggttcaactccttcctaacacatgcattgagatagctggggcaatgctactatttactatccatactgtccaaaaaa

SEQ ID NO: 36 - DNA, 2301 bp, Influenza virus
aatggatgtcaatccgacattacttttcttaaaagtgccagcacaaaatgctataagcacaacttttccttatactggtgaccctccttacagccatgggacaggaacagggtacaccatggatacagtcaacaggacacatcagtactcagaaagaggaagatggacaaaaaataccgaaactggagcaccgcaactcaacccaattgatgggccactaccaaaagacaatgaaccaagtggctatgcccaaacagattgtgtattagaagcaatggctttccttgaggaatcccatcctggtatttttgaaaactcttgtattgaaacaatggaggttgttcagcaaacaagggtggacaaactgacacaaggcagacagacctatgactggactctaaataggaaccagcctgctgccacagcattggccaacactatagaagtgttcagatcaaacggcctcatagcaaatgaatctgggaggctaatagacttccttaaagatgtaatggagtcgatggacagagacgaagtagagatcacaactcattttcaaagaaagaggagagtgagagacaatgtaactaaaaaaatggtgacccaaagaacaataggcaaaaagaaacataaattagacaaaagaagttacctaattagggcattaaccctgaacacaatgaccaaagatgctgagagggggaaactaaaacgcagagcaattgcaaccccaggaatgcaaataagggggtttgtatactttgttgagacactggcaagaagcatatgtgaaaagcttgaacaatcagggttgccagttggaggaaatgaaaagaaagcaaagttagcaaatgttgtaaggaagatgatgaccaactcccaggacactgaaatttctttcaccatcactggagataacacaaaatggaacgaaaatcaaaaccctagaatgttcttggccatgatcacatatataaccaaaaatcagcctgaatggttcagaaatattctaagtattgctccaataatgttttcaaacaaaatggcgagactaggtaaggggtacatgtttgaaagcaagagtatgaaactgagaactcaaatacctgcagagatgctagccaacatagatttgaaatatttcaatgattcaactaaaaagaaaattgaaaaaatccggccattattaatagatggaactgcatcattgagtcctggaatgatgatgggcatgttcaatatgttaagcaccgtcttgggcgtctccattctgaatcttgggcaaaagagatacaccaagactacttactggtgggatggtcttcaatcgtctgatgattttgctctgattgtgaatgcacccaactatgcaggaattcaagctggagttgacaggttttatcgaacctgtaagctgctcggaattaatatgagcaaaaagaagtcttacataaacagaacaggtacctttgagttcacgagctttttctatcgttatgggtttgttgccaatttcagcatggagcttcctagttttggggtgtctggggtcaatgaatctgcagacatgagtattggagtcactgtcatcaaaaacaatatgataaacaatgaccttggcccagcaactgctcaaatggcccttcagttatttataaaagattacaggtacacgtatcgatgccacagaggtgacacacaaatacaaacccggagatcatttgagataaagaaactatgggaccaaacccgctccaaagctgggctgttggtctctgatggaggccccaatttatataacattagaaatctccatattcctgaagtctgcttgaaatgggagttgatggatgaggattaccaggggcgtttatgcaacccattgaacccgtttgtcagtcataaagagattgaatcagtgaacaatgcagtgatgatgccggcacatggtccagccaaaaatatggagtatgacgctgttgcaacaacacactcctgggttcccaaaaggaatcgatccattttgaatacgagccaaagggggatacttgaggatgagcaaatgtatcagaggtgctgcaatttatttgaaaaattcttcccaagtagctcatacagaagaccagttggaatatccagtatggtagaggctatggtttccagagcccgaattgatgcacggattgatttcgaatctggaaggataaaaaaagaggaattcgctgagatcatgaagacctgttccaccattgaagacctcagacggcaaaaatagggaatttggcttgtccttcatgaaaa

SEQ ID NO: 37 - DNA, 1527 bp, Influenza virus
atcactcactgagtgacatcaaagtcatggcgtcccaaggcaccaaacggtcttacgaacagatggagactgatggggaacgccagaatgcaactgaaatcagagcatccgtcggaagaatgattggtggaattgggcgattctacatccaaatgtgcaccgagcttaaactcaatgattatgagggacgactgatccagaacagcttgacaatagagagaatggtgctctctgcttttgatgagaggaggaataaatatctggaagaacatcccagcgcggggaaagatcctaagaaaactggaggacccatatacaagagagtagatggaaagtgggtgagggaactcgtcctttatgacaaagaagaaataaggcggatttggcgccaagccaacaatggtgatgatgcaacggctggtttgactcacattatgatctggcattctaatttgaatgatacaacttaccagaggacaagagctcttgtccgcaccggaatggatcccaggatgtgctctttgatgcaaggttcaactctccctagaagatctggagcagcaggcgctgcagtcaaaggagttgggacaatggtgttggagttaatcaggatgatcaaacgtgggatcaatgaccgaaacttctggaggggtgagaatggaagaaaaacaaggattgcttatgagagaatgtgcaacattctcaaaggaaaatttcaaacagctgcacaaaaagcaatgatggatcaagtgagagaaagccggaacccaggaaatgctgagatcgaagatctcacttttctggcacggtctgcactcatattaagagggtcagttgctcacaagtcttgcctgcctgcctgtgtgtatggaccagccgtagccagtgggtacgacttcgaaaaagagggatactctttggtaggggtagacccttttaaactgcttcaaaccagtcaggtatacagcctaatcagaccaaacgagaatcccgcacacaagagtcagttggtgtggatggcatgcaattctgctgcatttgaagatctaagagtgtcaagcttcatcagagggacaagagtacttccaagggggaagctctccactagaggagtacaaattgcttcaaatgaaaacatggatgctattgtatcaagtactcttgaactgagaagcagatactgggccataagaaccagaagtggagggaacactaatcaacaaagggcctctgcgggccaaatcagcacacaacctacgttttctgtgcagagaaacctcccatttgacaaaacaaccatcatggcagcattcactgggaatacggagggaagaacatcagacatgagggcagaaatcataaagatgatggaaagtgcaagaccagaagaagtgtccttccaggggcggggagtctttgagctctcggacgaaagggcaacgaacccgatcgtgccctcctttgacatgagtaatgaaggatcttatttcttcggagacaatgcagaggagtacgacaattaatgaa

SEQ ID NO: 38 - DNA, 984 bp, Influenza virus
gatgagtcttctaaccgaggtcgaaacgtacgttctctctatcgtcccgtcaggccccctcaaagccgagatcgcacagagacttgaaaatgtctttgctggaaagaataccgatcttgaggctctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaatggggatccaaataatatggacagagcagttaaactgtatcgaaagcttaagagggagataacattccatggggccaaagaaatagcactcagttattctgctggtgcacttgccagttgtatgggactcatatacaacaggatgggggctgtgaccaccgaatcagcatttggccttatatgcgcaacctgtgaacagattgccgactcccagcataagtctcataggcaaatggtaacaacaaccaacccattaataagacatgagaacagaatggttctggccagcactacagctaaggctatggagcaaatggctggatcgagtgaacaagcagctgaggccatggaggttgctagtcaggccaggcagatggtgcaggcaatgagagccattgggactcatcctagctctagcactggtctgaaaaatgatctccttgaaaatttgcaggcctatcagaaacgaatgggggtgcagatgcaacgattcaagtgatcctcttgttgttgccgcaagtataattgggattgtgcacctgatattgtggattattgatcgccttttttccaaaagcatttatcgtatctttaaacacggtttaaaaagagggccttctacggaaggagtaccagagtctatgagggaagaatatcgagaggaacagcagaatgctgtggatgctgacgatggtcattttgtcagcatagagctagagtaaa

SEQ ID NO: 39 - DNA, 844 bp, Influenza virus
atggattcccacactgtgtcaagctttcaggtagattgcttcctttggcatgtccgcaaacaagttgcagaccaagatctaggcgatgccccattccttgatcggcttcgccgagatcagaagtctctaaagggaagaggcagcactctcggtctgaacatcgaaacagccacttgtgttggaaagcaaatagtagagaggattctgaaagaagaatccgatgaggcatttaaaatgaccatggcctccgcacttgcttcgcggtacctaactgacatgactattgaagaaatgtcaagggactggttcatgctcatgcccaagcagaaagtggctggccctctttgtgtcagaatggaccaggcgataatggataagaacatcatactgaaagcgaatttcagtgtgatttttgaccggttggagaatctgacattactaagggctttcaccgaagagggagcaattgttggcgaaatttcaccattgccttctcttccaggacatactaatgaggatgtcaaaaatgcaattggggtcctcatcgggggacttgaatggaatgataacacagttcgagtctctgaaactctacagagattcgcttggagaagcagtaatgagactgggggacctccattcactccaacacagaaacggaaaatggcgggaacaattaggtcagaagtttgaagaaataagatggctgattgaagaagtgaggcataaattgaagacgacagagaatagttttgagcaaataacatttatgcaagcattacagctattgtttgaagtggaacaagagattagaacgttttcgtttcagcttatttaatgataa

SEQ ID NO: 40 - DNA, 1728 bp, Influenza virus
ccaaaatgaaagcaaaactactggtcctgttatgtacatttacagctacatatgcagacacaatatgtataggctaccatgccaacaactcaaccgacactgttgacacagtacttgagaagaatgtgacagtgacacactctgtcaacctacttgaggacagtcacaatggaaaactatgtctactaaaaggaatagccccactacaattgggtaattgcagcgttgccggatggatcttaggaaacccagaatgcgaattactgatttccaaggaatcatggtcctacattgtagaaacaccaaatcctgagaatggaacatgttacccagggtatttcgccgactatgaggaactgagggagcaattgagttcagtatcttcatttgagagattcgaaatattccccaaagaaagctcatggcccaaccacaccgtaaccggagtatcagcatcatgctcccataatgggaaaagcagtttttacagaaatttgctatggctgacggggaagaatggtttgtacccaaacctgagcaagtcctatgtaaacaacaaagagaaagaagtccttgtactatggggtgttcatcacccgcctaacatagggaaccaaagggccctctatcatacagaaaatgcttatgtctctgtagtgtcttcacattatagcagaagattcaccccagaaatagccaaaagacccaaagtaagagatcaggaaggaagaatcaactactactggactctgctggaacctggggatacaataatatttgaggcaaatggaaatctaatagcgccatggtatgcttttgcactgagtagaggctttggatcaggaatcatcacctcaaatgcaccaatggatgaatgtgatgcgaagtgtcaaacacctcagggagctataaacagcagtcttcctttccagaatgtacacccagtcacaataggagagtgtccaaagtatgtcaggagtgcaaaattaaggatggttacaggactaaggaacatcccatccattcaatccagaggtttgtttggagccattgccggtttcattgaaggggggtggactggaatggtagatgggtggtatggttatcatcatcagaatgagcaaggatctggctatgctgcagatcaaaaaagtacacaaaatgccattaacgggattacaaacaaggtgaattctgtaattgagaaaatgaacactcaattcacagctgtgggcaaagaattcaacaaattggaaagaaggatggaaaacttaaataaaaaagttgatgatgggtttctagacatttggacatataatgcagaattgttggttctactggaaaatgaaaggactttggatttccatgactccaatgtgaagaatctgtatgagaaagtaaaaagccaattaaagaataatgccaaagaaataggaaacgggtgttttgaattctatcacaagtgtaacaatgaatgcatggagagtgtgaaaaatggaacttatgactatccaaaatattccgaagaatcaaagttaaacagggagaaaattgatggagtgaaattggaatcaatgggagtctatcagattctggcgatctactcaactgtcgccagttccctggttcttttggtctccctgggggcaatcagcttctggatgtgttccaatgggtctttgcagtgtagaatatgcatctgagaccagaatttcagaaatataagaa

SEQ ID NO: 41 - DNA, 1414 bp, Influenza virus
aatgaatccaaatcaaaaaataataaccattggatcaatcagtatagcaatcggaataattagtctaatgttgcaaataggaaatattatttcaatatgggctagtcactcaatccaaactggaagtcaaaaccacactggagtatgcaaccaaagaatcatcacatatgaaaacagcacctgggtgaatcacacatatgttaatattaacaacactaatgttgttgctggaaaggacaaaacttcagtgacattggccggcaattcatctctttgttctatcagtggatgggctatatacacaaaagacaacagcataagaattggctccaaaggagatgtttttgtcataagagaacctttcatatcatgttctcacttggaatgcagaaccttttttctgacccaaggtgctctattaaatgacaaacattcaaatgggaccgttaaggacagaagtccttatagggccttaatgagctgtcctctaggtgaagctccgtccccatacaattcaaagtttgaatcagttgcatggtcagcaagcgcatgccatgatggcatgggctggttaacaatcggaatttctggtccagacaatggagctgtggctgtactaaaatacaacggcataataactgaaaccataaaaagttggaaaaagcgaatattaagaacacaagagtctgaatgtgtctgtgtgaacgggtcatgtttcaccataatgaccgatggcccgagtaatggggccgcctcgtacaaaatcttcaagatcgaaaaggggaaggttactaaatcaatagagttgaatgcacccaattttcattatgaggaatgttcctgttacccagacactggcacagtgatgtgtgtatgcagggacaactggcatggttcaaatcgaccttgggtgtcttttaatcaaaacctggattatcaaataggatacatctgcagtggggtgttcggtgacaatccgcgtcccaaagatggagagggcagctgtaatccagtgactgttgatggagcagacggagtaaaggggttttcatacaaatatggtaatggtgtttggataggaaggactaaaagtaacagacttagaaaggggtttgagatgatttgggatcctaatggatggacagataccgacagtgatttctcagtgaaacaggatgttgtggcaataactgattggtcagggtacagcggaagtttcgttcaacatcctgagttaacaggattggactgtataagaccttgcttctgggttgagttagtcagaggactgcctagagaaaatacaacaatctggactagtgggagcagcatttctttttgtggcgtaaatagtgatactgcaaactggtcttggccagacggtgctgagttgccgttcaccattgacaagtag

SEQ ID NO: 42 - DNA, 2220 bp, Influenza virus
agcgaaagcaggtactgattcgaaatggaagattttgtgcgacaatgcttcaatccgatgattgtcgagcttgcggaaaaggcaatgaaagagtatggagaggacctgaaaatcgaaacaaacaaatttgcagcaatatgcacccacttggaagtatgcttcatgtattcagattttcatttcatcaatgagcaaggcgaatcaataatagtagagcctgaggacccaaatgcacttttaaaacacagatttgagataatagaggggcgagatcgtacaatggcatggacagttgtaaacagtatttgcaacaccacaggagctgagaaaccaaagtttctgccagatctgtatgattacaaagagaataggttcatcgaaattggagtgacaaggagagaagttcacatatactatctggaaaaggccaacaaaattaaatctgagaagacacatattcacattttctcatttactggcgaagaaatggccacaaaggccgattacactctcgatgaagaaagcagggctagaattaaaaccagactattcaccataaggcaagaaatggcaagcagaggtctttgggactcctttcgtcagtccgaaagaggcgaagagacaattgaagaaaggtttgaaatcacagggacaatgcgcaggctcgctgatcaaagccttccgccgaacttctcctgcattgagaattttagagcctatgtggatggatttgaaccgaacggctacattgagggcaagctttctcaaatgtccaaagaagtaaatgctaaaattgagccttttttgaaaacaacacctcgaccaattagacttccgaatgggcctccttgttttcagcggtcaaaattcctgctgatggattctttaaaattaagcattgaggatccaaatcatgaaggggagggaataccactatatgatgcaatcaagtgtatgagaacattctttggatggaaagaacccactgttgtcaagccacacgagaagggaataaatccgaattatctgctgtcgtggaagcaggtgttggaagagctgcaggacattgagagtgaggagaagattccaagaacaaaaaacatgaaaaaaacgagtcagttaaagtgggcacttggtgagaacatggcaccagagaaggtggattttgatgactgtaaagatataagcgatttgaagcaatatgatagtgacgaacctgaattaaggtcattttcaagttggatccagaatgagttcaacaaggcatgcgagctgaccgattcaatctggatagagctcgatgagattggagaagatgtggccccgattgaacacattgcaagcatgagaagaaattacttcacagctgaggtgtcccattgcagagccactgaatatataatgaaaggggtatacattaatactgctttgcttaatgcatcctgtgcagcaatggatgatttccaactaattcctatgataagcaaatgtagaactaaagagggaaggagaaagaccaatttgtacggcttcatcataaaaggaagatctcacttaaggaatgataccgatgtggtaaactttgtgagcatggagttttccctcactgacccaagacttgagccacacaaatgggagaagtactgtgttcttgagataggagatatgcttctaaggagtgcaataggccaagtgtcaaggcccatgttcttgtatgtaagaacaaatggaacctcaaaaattaaaatgaaatggggaatggagatgaggcgttgcctcctccaatccctccaacaaatagagagcatgattgaagctgagtcctctgtcaaggagaaagacatgacaaaagagttttttgagaatagatcagaaacatggcccattggagagtcaccaaaaggagtggaagaaggttccattgggaaagtatgcaggacactattggctaaatcagtattcaatagtctgtatgcatctccacaattagaaggattttcagctgagtcaagaaagttgctccttattgttcaggctcttagggacaatctggaacctgggacctttgatcttgggggactatatgaagcaattgaggagtgcctgattaatgatccctgggttttgcttaatgcttcttggttcaactccttcctaaaacatgcattgagatagctgaggcaatgctactatttgttatccatactgtccaaaaaagta

SEQ ID NO: 43 - DNA, 2341 bp, Influenza virus
agcgaaagcaggcaaaccatttgaatggatgtcaatccgacattacttttcttaaaagtgccagcacaaaatgctataagcacaacttttccttatactggtgaccctccttacagccatggaacaggaacaggatacaccatggatacagtcaacaggacacatcagtactcagaaagaggaagatggacgaaaaataccgaaactggagcaccgcaactcaacccaattgatgggccactaccagaagacaatgaaccaagtggctatgcccaaacagattgtgtattagaggcaatggctttccttgaagaatcccatcctggtatttttgaaaactcttgtattgaaacaatggaggttgttcagcaaacaagggtggacaaactgacacaaggcagacaaacctatgactggactctaaataggaaccagcctgctgccacagcattggcaaacaccatagaagtattcagatcaaatggcctcatagcaaatgaatctggaaggctaatagacttccttaaagatgtaatggagtcgatggacagagacgaagtagaggtcacaactcattttcaaagaaagaggagagtgagagacaatgtaactaaaaaaatggtgacccaaagaacaataggaaaaaagaaacataaattagacaaaagaagttacctaattagggcattaaccctgaacacaatgaccaaagatgctgagagggggaaactaaaacgcagagcaattgcaaccccaggaatgcaaataagggggtttgtatactttgttgagacactggcaagaagcatatgtgaaaagcttgaacaatcagggttgccagttggaggaaatgagaagaaagcaaagttagcaaatgttgtaaggaagatgatgaccaactcccaggacactgaaatttcttttaccatcactggagataacacaaaatggaacgaaaatcaaaaccctagaatgttcttggccatgatcacatatataaccaaagatcagcctgaatggttcagaaatattctaagtattgctccaataatgttttcaaacaaaatggcgagactaggtagggggtatatgtttgaaagcaagagtatgaaactgagaacccaaatacctgcagagatgctagccaacatagatttgaaatatttcaatgattcaactaaaaagaaaattgaaaaaattcgaccattattaatagatggaactgcatcattgagtcctggaatgatgatgggcatgttcaatatgttaagcaccgtcttgggcgtttccattctgaatcttgggcaaaaaagatacaccaagactacttactggtgggatggtcttcaatcgtctgatgattttgctttgattgtgaatgcacccaattatgcaggaattcaagctggagttgacaggttttatcgaacctgtaagctgctcggaattaatatgagcaaaaagaagtcttacataaacagaacaggtacctttgaattcacgagctttttctatcgttatgggtttgttgccaatttcagcatggagcttcctagttttggggtgtctggggtcaatgaatctgcagacatgagtattggagtcactgtcatcaaaaacaatatgataaacaatgaccttggcccagcaactgctcaaatggcccttcagttatttataaaagattacaggtacacttatcgatgccacagaggtgacacacaaatacaaacccggagatcatttgaaataaagaaactatgggaccaaacccgctccaaagctgggctgttggtctctgatggaggccccaatttatataacattaggaatctacatattcctgaagtctgcttgaaatgggagttgatggatgaggattaccaggggcgtttatgcaacccattgaacccgtttgtcagccataaagagattgaatcagtgaacaatgcagtgataatgccggcacatggtccagccaaaaatatggagtatgacgctgttgcaacaacacactcttgggtccccaaaagaaatcgatccattttaaacacgagccaaagagggatacttgaagatgagcaaatgtaccaaaggtgctgcaatttatttgaaaaattcttcccaagtagctcatacagaagaccagttggaatatccagtatggtagaggctatggtttcaagagcccgaattgatgcacggattgatttcgaatctggaaggataaagaaagaggaattcgctgagatcatgaagacctgttccaccattgaagacctcagacggcaaaaatagggaatttggcttgtccttcatgaaaaaatgccttgtttctact

SEQ ID NO: 44 - DNA, 2341 bp, Influenza virus
agcgaaagcaggtcaattatattcaatatggaaagaataaaagagctaaggaatctgatgtcacaatctcgcactcgcgagatacttaccaaaactactgtagaccacatggccataataaagaaatacacatcaggaagacaggagaaaaacccatcacttaggatgaaatggatgatggcaatgaaatacccaattacagctgataaaaggataacggaaatgattcctgaaagaaatgagcaaggacagacactatggagtaaagtgaatgatgccggatcagaccgagtgatgatatcacccctagctgtgacatggtggaacagaaatggaccagtggcaaacactatccactatccaaaaatctacaaaacttactttgaaaaggttgaaaggttaaaacatggaacctttggccctgtacactttagaaaccaagtcaaaatacgccgaagagtcgacataaatcctggtcatgcagacctcagcgccaaggaggcacaggatgtaattatggaagttgttttccctaatgaagtgggagccagaatactaacatcagaatcgcaattaacgataactaaggagaaaaaagaggaactccagaattgcaaaatttcccctttgatggttgcatacatgttagagagggaacttgtccgcaaaacaagatttctcccggttgcaggtggaacaagcagtgtgtacattgaagttttgcatttaacacaggggacatgctgggagcagatgtacactccaggtggggaggtgaggaatgatgatgttgatcaaagcctaattattgctgctaggaacatagtgagaagagctgcagtatcagcagatccactagcatctttattagaaatgtgccatagcacacagattggtggaacaaggatggtggatattctcaggcaaaatccaacagaagaacaagctgtggacatatgcaaagcagcaatggggctgagaatcagttcatccttcagttttggcggattcacatttaagagaacaagtggatcgtcagtcaaaagggaggaagaagtgctaacgggcaatctgcaaacattgaagctaactgtgcatgagggatatgaagaattcacaatagttgggaaaaaggcaacagctatactcagaaaagcaaccaggagattgattcaactaatagtgagtggaagagacgaacagtcaatagtcgaagcaatagttgtagcaatggtattctcacaagaagattgcatggtaaaagcggttagaggtgatctgaatttcgttaatagagcgaatcagcggttgaatcccatgcatcaacttttgagacattttcagaaggatgctaaagtacttttcctaaattggggaattgaacatattgacaatgtgatgggaatgattgggatattacctgatatgactccaagtaccgagatgtcaatgagaggagtgagagtcagcaaaatgggtgtagatgaatactccaatgctgaaagggtagtggtaagcattgaccgttttttgagggtccgggaccaaagaggaaatgtattactgtctccagaggaagtcagtgaaacacaaggaacagagaaactgacaataacttactcttcatcattgatgtgggagattaatggccctgagtcagtgttgatcaatacctaccaatggatcatcagaaactgggagactgttaaaattcagtggtctcagaaccctacaatgctatacaataaaatggaatttgagccatttcaatctctagtccccaaggccattagaggccaatacagtgggtttgttagaactctatttcaacaaatgagggatgtgctcgggacctttgacacaactcagataataaaacttcttccctttgcagccgctccaccaaagcaaagtagaatgcaattctcgtcattaactgtgaatgtgaggggatcaggaatgagaatacttgtaaggggtaattctccagtattcaactacaacaagaccactaagagactcacaatcctcggaaaggatgctggcactttaactgaagacccagatgaaggcacagctggagtggaatctgctgttttaaggggattcctcattctaggcaaagaagatagaagatatgggccagcattaagcatcagtgaattgagcaaccttgcgaaaggggagaaagctaatgtgctaattgggcaaggggatgtagtgttggtaatgaaacgaaaacgggactctagcatacttactgacagccagacagcgaccaaaagaattcggatggccatcaattaatttcgaataatttaaaaacgaccttgtttctact

SEQ ID NO: 45 - DNA, 1565 bp, Influenza virus
agcaaaagcagggtagataatcactcactgagtgacatcaaagtcatggcgtcccaaggcaccaaacggtcttacgaacagatggagactgatggggaacgccagaatgcaactgaaatcagagcatccgtcggaagaatgattgggggaattgggcgattctacatccaaatgtgcaccgagcttaagctcaatgattatgagggacgactgatccagaacagcttaacaatagagagaatggtgctttctgcttttgatgagaggagaaataaatatctggaagaacatcccagcgcagggaaagatcctaagaaaactggaggacccatatacaagagagtagatggaaagtgggtgagggaactcgtcctttatgacaaagaagaaataaggcggatttggcgccaagccaacaatggtgatgatgcaacagctggtttgactcacattatgatctggcattctaatttgaatgatacaacttaccagaggacaagagctcttgtccgcaccggaatggatcccaggatgtgctctttgatgcaaggttcaactctccctagaagatctggagcagcaggcgctgcagtcaaaggagttgggacaatggtattggagttaatcaggatgatcaaacgtgggatcaacgaccgaaacttctggaggggtgagaatgggagaaaaacaaggattgcttatgagagaatgtgcaacattctcaaaggaaaatttcaaacagctgcacaaaaagcaatgatggatcaagtgagagaaagccggaacccaggaaatgctgagatcgaagatctcacttttctggcacggtctgcactcatattgagaggatcagttgctcacaagtcttgcctgcctgcttgtgtgtatggaccagccgtagccagtgggtatgacttcgaaaaagagggatactctttggtgggagtagaccctttcaaactgcttcaaaccagtcaggtatacagcctaattagaccaaacgagaatcccgcacacaagagccagttggtgtggatggcatgcaattctgctgcatttgaagatctaagagtgtcaagcttcatcagagggacaagagtacttccaagggggaagctctccactagaggagtacaaattgcttcaaatgaaaacatggatgctattgtctcaagtactcttgaactgagaagcagatactgggccataagaaccagaagtggagggaacaccaatcaacaaagggcctctgcgggccaaatcagcacacaacctacgttttctgtgcagagaaacctcccatttgacaaaacaaccatcatggcagcattcactgggaatacagagggaagaacatcagacatgcgggcagaaatcataaagatgatggaaagtgcaagaccagaagaagtgtccttccagggacggggagtctttgagctctcggacgaaagggcaacgaacccgatcgtgccctcctttgacatgagtaatgaaggatcttatttcttcggagacaatgcagaggagtacgacaattaatgaaaaatacccttgtttctact

SEQ ID NO: 46 - DNA, 1027 bp, Influenza virus
agcaaaagcaggtagatattgaaagatgagtcttctaaccgaggtcgaaacgtacgttctctctatcgtcccatcaggccccctcaaagccgagatcgcacagagacttgaagatgtatttgctggaaagaataccgatcttgaggctctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaatggggatccaaataatatggacaaggctgtcaaactgtatcgaaagcttaagagggagataacattccatggggccaaagaaatagcactcagttattctgctggagcacttgccagttgtatgggactcatatacaacaggatgggggctgtgaccaccgaatcagcatttggccttatatgtgcaacctgtgaacagattgccgactcccagcataagtctcataggcaaatggtaacaacaaccaatccattaataagacatgagaacagaatggttctggccagcactacagctaaggctatggagcaaatggctggatcgagtgaacaagcagctgaggccatggaggttgctagtcaggccaggcagatggtgcaggcaatgagagccattgggactcatcctagctctagcactggtctgaaaaatgatctccttgaaaatttgcaggcctatcagaaacgaatgggggtgcagatgcaacgattcaagtgatcctcttgttgttgccgcaagtataattgggattgtgcacctgatattgtggattattgatcgccttttttccaaaagcatttatcgtatttttaaacacggtttaaaaagagggccttctacggaaggagtaccggagtctatgagggaagaatatcgagaggaacagcagaatgctgtggatgctgacgatggtcattttgtcagcatagagctagagtaaaaaactaccttgtttctact

SEQ ID NO: 47 - DNA, 889 bp, Influenza virus
agcaaaagcagggtggcaaagacataatggattcccacactgtgtcaagctttcaggtagattgtttcctttggcatgtccgcaaacaagttgcagaccaagatctaggcgatgcccccttccttgatcggcttcgccgagatcagaagtctctaaagggacgaggcaacactctcggtctgaacatcgaaacagccacttgtgttggaaagcaaatagtagagaggattctgaaagaagaatccgatgagacatttagaatgaccatggcctccgcacttgcttcgcggtacctaactgacatgactgttgaagaaatgtcaagggactggttcatgctcatgcccaagcagaaagtggctggccctctttgtgtcagaatggaccaggcgataatggataagaacatcatactgaaagcgaacttcagtgtgatttttgaccggttggagaatctgacattactaagggctttcaccgaagagggagcaattgttggcgaaatttcaccattgccttcttttccaggacatactaatgaggatgtcaaaaatgcaattggggtcctcatcgggggacttgaatggaatgataacacagttcgagtctctgaagctctacagagattcgcttggagaagcagtaatgagactgggggacctccattcactacaacacagaaacggaaaatggcgggaacaattaggtcagaagtttgaagaaataagatggctgattgaagaagtgaggcataaattgaagacgacagagagtagttttgaacaaataacatttatgcaagcattacagctattgtttgaagtggaacaagagattagaacgttctcgtttcagcttatttaatgataaaaacacccttgtttctact

SEQ ID NO: 48 - DNA, 1775 bp, Influenza virus
agcgaaagcaggggaaaataaaagcaaccaaaatgaaagtaaaactactggttctgttatgtacatttacagctacatatgcagacacaatatgtataggctaccatgccaacaactcaaccgacactgttgacacagtacttgagaagaatgtaacagtgacacactctgtcaacctacttgaggacagtcacaatggaaaactatgtctactaaaaggaatagccccactacaattgggtaattgcagcgttgccggatggatcttaggaaacccagaatgcgaattactgatttccaaggaatcatggtcctacattgtagaaacaccaaatcctgagaatggaacatgttacccagggtatttcgccgactatgaggaactgagggagcaattgagttcagtatcttcatttgaaaggttcgaaatattccccaaagagagctcatggcccaaccacaccgtaaccggagtatcagcatcatgctcccataacgggaaaagcagtttttacagaaatttgctatggctgacggggaagaatggtttgtacccaaacctgagcaagtcctatgcaaacaacaaagagaaagaagtccttgtactatggggtgttcatcacccgcctaacataggggaccaaagggccctctatcatacagaaaatgcttatgtctctgtagtgtcttcacattatagcagaagattcaccccagaaatagccaaaagacccaaggtgagagaccaggaaggaagaatcaactactactggactctgctggaacccggggatacaataatatttgaggcaaatggaaatctaatagcgccaaggtatgctttcgcactgagtagaggcttgggatcaggaatcatcacctcaaatgcaccaatggatgaatgtgatgcaaagtgtcaaacacctcagggagctataaacagcagtcttcctttccagaatgtacacccagtcacaataggagagtgtccaaagtatgtcaggagtgcaaaattaaggatggttacaggactaaggaacatcccatccattcaatccagaggtttgtttggagcaattgccggtttcattgaaggggggtggactggaatggtagatggttggtatggttatcatcatcagaatgagcaaggatctgggtatgctgcagatcaaaaaagcacacaaaatgccattaacgggattacaaacaaggtgaattctgtaattgagaaaatgaacactcaattcacagctgtgggcaaagaattcaacaaattggaaagaaggatggaaaacttaaataaaaaagttgatgatgggtttctagacatttggacctataatgcagaattgttggttctactggaaaatgaaaggactttggatttccatgactccaacgtgaagaatctgtatgagaaagtaaaaagccaattaaagaataatgccaaagaaataggaaacgggtgttttgaattctatcacaagtgtaacgatgaatgcatggagagtgtgaaaaatggaacttatgactatccaaaatattccgaagaatcaaagttaaacagagagaaaattgatggagtgaaattggaatcaatgggagtctatcagattctggcgatctactcaacagtcgccagttccctggttcttttggtctccctgggggcaatcagcttctggatgtgttccaatgggtctttgcagtgtagaatatgcatctaagaccagaatttcagaaatataaggaaaaacacccttgtttctact

SEQ ID NO: 49 - DNA, 1462 bp, Influenza virus
agcaaaagcaggagtttaaaatgaatccaaatcaaaaaataataaccattggatcaatcagtatagcaatcggaataattagtctaatgttgcaaataggaaatattatttcaatatgggctagtcactcaatccaaactggaagtcaaaaccacactggaatatgcaaccaaaaaatcatcacatatgaaaacagcacctgggtgaatcacacatatgttaatattaacaacactaatgttgttgctggaaaggacaaaacttcagtgacactggccggcaattcatctctttgtcctatcagtggatgggctatatacacaaaagacaacagcataagaattggctccaaaggagatgtttttgtcataagagaacctttcatatcatgttctcacttggaatgcagaaccttttttctgacccaaggtgctctattaaatgacaaacattcaaatggaaccgttaaggacagaagtccttatagggccttaatgagctgtcctctaggtgaagccccgtcaccatacaattcaaagtttgaatcagttgcatggtcagcaagcgcatgccatgatggcaagggctggttaacaatcggaatttctggtccagacaatggagctgtggctgtactaaaatacaacggaataataactgaaaccataaaaagttgggaaaagcgaatattgagaacacaagagtctgaatgtgtttgtgtgaacgggtcatgtttcaccataatgaccgatggcccgagtaatggggccgcctcgtacaaaatcttcaagatcgaaaaggggaaggttactaaatcaacagagttgaatgcacccaattttcattatgaggaatgttcctgttacccagacactggcacagtgatgtgtgtatgcagggacaactggcatggttcaaatcgaccttgggtatcttttaatcaaaacttggattatcaaataggatacatctgcagtggagtgttcggtgacaatccgcgtcccaaagatgggaagggcagctgtaatccagtgactgttgatggagcagacggagttaaggggttttcatacaaatatggtaatggtgtttggataggaaggactaaaagtaacagacttagaaaggggtttgagatgatttgggatcctaatggatggacagataccgacagtgatttctcagtgaaacaggatgttgtggcaataactgattggtcagggtacagcggaagtttcgtccaacatcctgagttaacaggattggactgtataagaccttgcttctgggttgagttagtcagaggactgcctagagaaaatacaacaatctggactagtgggagcagcatttctttttgtggcgttgatagtgatactgcaaattggtcttggccagacggtgctgagttgccgttcaccattgacaagtagctcgttgaaaaaaactccttgtttctact

SEQ ID NO: 50 - protein, 566 aa, Influenza virus
MKAKLLVLLCALSATDADTICIGYHANNSTDTVDTVLEKNVTVTHSVNLLEDNHNGKLCKLKGIAPLQLGKCSIAGWILGNPECESLFSKKSWSYIAETPNSENGTCYPGYFADYEELREQLSSVSSFERFEIFPKESSWPKHNVTKGVTAACSHKGKSSFYRNLLWLTEKNGSYPNLSKSYVNNKEKEVLVLWGVHHPSNIEDQKTIYRKENAYVSVVSSHYNRRFTPEIAKRPKVRNQEGRINYYWTLLEPGDTIIFEANGNLIAPWYAFALSRGFGSGIITSNASMDECDAKCQTPQGAINSSLPFQNVHPVTIGECPKYVRSTKLRMVTGLRNIPSIQSRGLFGAIAGFIEGGWTGMIDGWYGYHHQNEQGSGYAADQKSTQNAINGITNKVNSIIEKMNTQFTAVGKEFNKLEKRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 51 - protein, 470 aa, Influenza virus
MNPNQKIITIGSICMTIGIISLILQIGNIISIWVSHSIQTGSQNHTGICNQRIITYENSTWVNQTYVNINNTNVVAGKDTTSVTLAGNSSLCPIRGWAIYSKDNSIRIGSKGDVFVIREPFISCSHLECRTFFLTQGALLNDKHSNGTVKDRSPYRALMSCPIGEAPSPYNSRFESVAWSASACHDGMGWLTIGISGPDDGAVAVLKYNGIITETIKSWRKRILRTQESECVCVNGSCFTIMTDGPSNGPASYRIFKIEKGKITKSIELDAPNSHYEECSCYPDTGTVMCVCRDNWHGSNRPWVSFNQNLDYQIGYICSGVFGDNPRPKDGKGSCDPVTVDGADGVKGFSYRYGNGVWIGRTKSNSSRKGFEMIWDPNGWTDTDSNFLVKQDVVAMTDWSGYSGSFVQHPELTGLDCMRPCFWVELVRGRPREGTTVWTSGSSISFCGVNSDTANWSWPDGAELPFTIDK

SEQ ID NO: 52 - protein, 469 aa, Influenza virus
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 53 - protein, 716 aa, Influenza virus; identical to SEQ ID NO: 17

SEQ ID NO: 54 - protein, 757 aa, Influenza virus
MDVNPTLLFLKVPAQNAISTTFPYTGDPPYSHGTGTGYTMDTVNRTHQYSERGRWTKNTETGAPQLNPIDGPLPKDNEPSGYAQTDCVLEAMAFLEESHPGIFENSCIETMEVVQQTRVDKLTQGRQTYDWTLNRNQPAATALANTIEVFRSNGLIANESGRLIDFLKDVMESMDRDEVEVTTHFQRKRRVRDNVTKKMVTQRTIGKKKHKLDKRSYLIRALTLNTMTKDAERGKLKRRAIATPGMQIRGFVYFVETLARSICEKLEQSGLPVGGNEKKAKLANVVRKMMTNSQDTEISFTITGDNTKWNENQNPRMFLAMITYITKNQPEWFRNILSIAPIMFSNKMARLGKGYMFESKSMKLRTQIPAEMLANIDLKYFNDSTKRKIEKIRPLLIDGTASLSPGMMMGMFNMLSTVLGVSILNLGQKRYTKTTYWWDGLQSSDDFALIVNAPNYAGIQAGVDRFYRTCKLLGINMSKKKSYINRTGTFEFTSFFYRYGFVANFSMELPSFGVSGVNESADMSIGVTVIKNNMINNDLGPATAQMALQLFIKDYRYTYRCHRGDTQIQTRRSFEIKKLWDQTRSKAGLLVSDGGPNLYNIRNLHIPEVCLKWELMDEDYQGRLCNPSNPFVSHKEIESVNNAVMMPAHGPAKNMEYDAVATTHSWVPKRNRSILNTSQRGILEDEQMYQRCCNLFEKFFPSSSYRRPVGISSMVEAMVSRARIDARIDFESGRIKKEEFAEIMKTCSTIEDLRRQK

SEQ ID NO: 55 - protein, 759 aa, Influenza virus
MERIKELRNLMSQSRTREILTKTTVDHMAIIKKYTSGRQEKNPSLRMKWMMAMKYPITADKRITEMIPERNEQGQTLWSKVNDAGSDRVMISPLAVTWWNRNGPVASTIHYPKIYKTYFEKVERLKHGTFGPVHFRNQVKIRRRVDINPGHADLSAKEAQDVIMEVVFPNEVGARILTSESQLTITKEKKEELQNCKISPLMVAYMLERELVRKTRFLPVAGGTSSVYIEVLHLTQGTCWEQMYTPGGEVRNDDVDQSLIIAARNIVRRAAVSADPLASLLEMCHSTQIGGTRMVDILRQNPTEEQAVDICKAAMGLRISSSFSFGGFTFKRTSGSSVKREEEVLTGNLQTLKLTVHEGYEEFTMVGKRATAILRKATRRLIQLIVSGRDEQSIVEAIVVAMVFSQEDCMVKAVRGDLNFVNRANQRLNPMHQLLRHFQKDAKVLFLNWGIEPIDNVMGMIGILPDMTPSTEMSMRGVRVSKMGVDEYSNAERVVVSIDRFLRVRDQRGNVLLSPEEVSETQGTEKLTITYSSSMMWEINGPESVLINTYQWIIRNWETVKIQWSQNPTMLYNKMEFEPFQSLVPKAIRGQYSGFVRTLFQQMRDVLGTFDTTQIIKLLPFAAAPPKQSRMQFSSLTVNVRGSGMRILVRGNSPVFNYNKTTKRLTVLGKDAGTLTEDPDEGTAGVESAVLRGFLILGKEDRRYGPALSINELSNLAKGEKANVLIGQGDVVLVMKRKRDSSILTDSQTATKRIRMAIN

SEQ ID NO: 56 - protein, 498 aa, Influenza virus
MASQGTKRSYEQMETDGERQNATEIRASVGRMIGGIGRFYIQMCTELKLNDYEGRLIQNSLTIERMVLSAFDERRNKYLEEHPSAGKDPKKTGGPIYKRVDGKWVRELVLYDKEEIRRIWRQANNGDDATAGLTHIMIWHSNLNDTTYQRTRALVRTGMDPRMCSLMQGSTLPRRSGAAGAAVKGVGTMVLELIRMIKRGINDRNFWRGENGRKTRIAYERMCNILKGKFQTAAQKAMMDQVRESRNPGNAEIEDLTFLARSALILRGSVAHKSCLPACVYGPAVASGYDFEKEGYSLVGVDPFKLLQTSQVYSLIRPNENPAHKSQLVWMACNSAAFEDLRVSSFIRGTRVLPRGKLSTRGVQIASNENMDAIVSSTLELRSRYWAIRTRSGGNTNQQRASAGQISTQPTFSVQRNLPFDKTTIMAAFTGNTEGRTSDMRAEIIKMMESARPEEVSFQGRGVFELSDERATNPIVPSFDMSNEGSYFFGDNAEEYDN

SEQ ID NO: 57 - protein, 252 aa, Influenza virus
MSLLTEVETYVLSIVPSGPLKAEIAQRLENVFAGKNTDLEALMEWLKTRPILSPLTKGILGFVFTLTVPSERGLQRRRFVQNALNGNGDPNNMDRAVKLYRKLKREITFHGAKEIALSYSAGALASCMGLIYNRMGAVTTESAFGLICATCEQIADSQHKSHRQMVTTTNPLIRHENRMVLASTTAKAMEQMAGSSEQAAEAMEVASQARQMVQAMRAIGTHPSSSTGLKNDLLENLQAYQKRMGVQMQRFK

SEQ ID NO: 58 - protein, 470 aa, Influenza virus
MNPNQKIITIGSISIAIGIISLMLQIGNIISIWASHSIQTGSQNHTGVCNQRIITYENSTWVNHTYVNINNTNVVAGKDKTSVTLAGNSSLCSISGWAIYTKDNSIRIGSKGDVFVIREPFISCSHLECRTFFLTQGALLNDKHSNGTVKDRSPYRALMSCPLGEAPSPYNSKFESVAWSASACHDGMGWLTIGISGPDNGAVAVLKYNGIITETIKSWKKRILRTQESECVCVNGSCFTIMTDGPSNGAASYKIFKIEKGKVTKSIELNAPNFHYEECSCYPDTGTVMCVCRDNWHGSNRPWVSFNQNLDYQIGYICSGVFGDNPRPKDGEGSCNPVTVDGADGVKGFSYKYGNGVWIGRTKSNRLRKGFEMIWDPNGWTDTDSDFSVKQDVVAITDWSGYSGSFVQHPELTGLDCIRPCFWVELVRGLPRENTTIWTSGSSISFCGVNSDTANWSWPDGAELPFTIDK

SEQ ID NO: 59 - protein, 716 aa, Influenza virus
MEDFVRQCFNPMIVELAEKAMKEYGEDLKIETNKFAAICTHLEVCFMYSDFHFINEQGESIVVELDDPNALLKHRFEIIEGRDRTMAWTVVNSICNTTGAGKPKFLPDLYDYKENRFIEIGVTRREVHIYYLEKANKIKSENTHIHIFSFTGEEMATKADYTLDEESRARIKTRLFTIRQEMANRGLWDSFRQSERGEETIEEKFEITGTMRRLADQSLPPNFSCLENFRAYVDGFEPNGCIEGKLSQMSKEVNAQIEPFLKTTPRPIKLPNGPPCYQRSKFLLMDALKLSIEDPSHEGEGIPLYDAIKCMKTFFGWKEPYIVKPHEKGINSNYLLSWKQVLSELQDIENEEKIPRTKNMKKTSQLKWALGENMAPEKVDFENCRDISDLKQYDSDEPELRSLSSWIQNEFNKACELTDSVWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRSAIGQISRPMFLYVRTNGTSKVKMKWGMEMRRCLLQSLQQIESMIEAESSVKEKDMTKEFFENKSEAWPIGESPKGVEEGSIGKVCRTLLAKSVFNSLYASPQLEGFSAESRKLLLVVQALRDNLEPGTFDLGGLYEAIEECLINDPWVLLNASWFNSFLTHALK

SEQ ID NO: 60 - protein, 757 aa, Influenza virus
MDVNPTLLFLKVPAQNAISTTFPYTGDPPYSHGTGTGYTMDTVNRTHQYSEKGKWTTNTETGAPQLNPIDGPLPEDNEPSGYAQTDCVLEAMAFLEESHPGIFENSCLETMEAVQQTRVDRLTQGRQTYDWTLNRNQPAATALANTIEVFRSNGLTANESGRLIDFLKDVMESMDKEEMEITTHFQRKRRVRDNMTKKMVTQRTIGKKKQRVNKRGYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYITKNQPEWFRNILSIAPIMFSNKMARLGKGYMFESKRMKLRTQIPAEMLASIDLKYFNESTRKKIEKIRPLLIDGTASLSPGMMMGMFNMLSTVLGVSILNLGQKKYTKTTYWWDGLQSSDDFALIVNAPNHEGIQAGVNRFYRTCKLVGINMSKKKSYINKTGTFEFTSFFYRYGFVANFSMELPSFGVSGINESADMSIGVTVIKNNMINNDLGPATAQMALQLFIKDYRYTYRCHRGDTQIQTRRSFELKKLWDQTQSRAGLLVSDGGPNLYNIRNLHIPEVCLKWELMDENYRGRLCNPLNPFVSHKEIESVNNAVVMPAHGPAKSMEYDAVATTHSWIPKRNRSILNTSQRGILEDEQMYQKCCNLFEKFFPSSSYRRPIGISSMVEAMVSRARIDARIDFESGRIKKEEFSEIMKICSTIEELRRQR

SEQ ID NO: 61 - protein, 759 aa, Influenza virus
MERIKELRNLMSQSRTREILTKTTVDHMAIIKKYTSGRQEKNPSLRMKWMMAMKYPITADKRITEMVPERNEQGQTLWSKMSDAGSDRVMVSPLAVTWWNRNGPVTSTVHYPKVYKTYFDKVERLKHGTFGPVHFRNQVKIRRRVDINPGHADLSAKEAQDVIMEVVFPNEVGARILTSESQLTITKEKKEELRDCKISPLMVAYMLERELVRKTRFLPVAGGTSSIYIEVLHLTQGTCWEQMYTPGGEVRNDDVDQSLIIAARNIVRRAAVSADPLASLLEMCHSTQIGGTRMVDILRQNPTEEQAVDICKAAMGLRISSSFSFGGFTFKRTSGSSVKKEEEVLTGNLQTLKIRVHEGYEEFTMVGKRATAILRKATRRLVQLIVSGRDEQSIAEAIIVAMVFSQEDCMIKAVRGDLNFVNRANQRLNPMHQLLRHFQKDAKVLFQNWGIEHIDSVMGMVGVLPDMTPSTEMSMRGIRVSKMGVDEYSSTERVVVSIDRFLRVRDQRGNVLLSPEEVSETQGTERLTITYSSSMMWEINGPESVLVNTYQWIIRNWEAVKIQWSQNPAMLYNKMEFEPFQSLVPKAIRSQYSGFVRTLFQQMRDVLGTFDTTQIIKLLPFAAAPPKQSRMQFSSLTVNVRGSGMRILVRGNSPVFNYNKTTKRLTILGKDAGTLIEDPDESTSGVESAVLRGFLIIGKEDRRYGPALSINELSNLAKGEKANVLIGQGDVVLVMKRKRDSSILTDSQTATKRIRMAIN

SEQ ID NO: 62 - protein, 498 aa, Influenza virus
MASQGTKRSYEQMETDGDRQNATEIRASVGKMIDGIGRFYIQMCTELKLSDYEGRLIQNSLTIEKMVLSAFDERRNKYLEEHPSAGKDPKKTGGPIYRRVDGKWMRELVLYDKEEIRRIWRQANNGEDATAGLTHIMIWHSNLNDATYQRTRALVRTGMDPRMCSLMQGSTLPRRSGAAGAAVKGIGTMVMELIRMVKRGINDRNFWRGENGRKTRSAYERMCNILKGKFQTAAQRAMVDQVRESRNPGNAEIEDLIFLARSALILRGSVAHKSCLPACVYGPAVSSGYNFEKEGYSLVGIDPFKLLQNSQVYSLIRPNENPAHKSQLVWMACHSAAFEDLRLLSFIRGTKVSPRGKLSTRGVQIASNENMDNMGSGTLELRSGYWAIRTRSGGNTNQQRASAGQTSVQPTFSVQRNLPFEKSTIMAAFTGNTEGRTSDMRAEIIRMMEGAKPEEVSFRGRGVFELSDEKATNPIVPSFDMSNEGSYFFGDNAEEYDN

SEQ ID NO: 63 - protein, 252 aa, Influenza virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVATTNPLIRHENRMVLASTTAKAMEQMAGSSEQAAEAMEIASQARQMVQAMRAIGTHPSSSTGLRDDLLENLQTYQKRMGVQMQRFK

SEQ ID NO: 64 - protein, 97 aa, Influenza virus
MSLLTEVETPIRNEWGCRCNDSSDPLVVAANIIGILHLILWILDRLFFKCVYRLFKHGLKRGPSTEGVPESMREEYRKEQQNAVDADDSHFVSIELE

SEQ ID NO: 65 - DNA, 846 bp, Influenza virus
aatggattccaacactgtgtcaagtttccaggtagattgctttctttggcatatccggaaacaagttgtagaccaagaactgagtgatgccccattccttgatcggcttcgccgagatcagaggtccctaaggggaagaggcaatactctcggtctagacatcaaagcagccacccatgttggaaagcaaattgtagaaaagattctgaaagaagaatctgatgaggcacttaaaatgaccatggtctccacacctgcttcgcgatacataactgacatgactattgaggaattgtcaagaaactggttcatgctaatgcccaagcagaaagtggaaggacctctttgcatcagaatggaccaggcaatcatggagaaaaacatcatgttgaaagcgaatttcagtgtgatttctgaccgactagagaccatagtattactaagggctttcaccgaagagggagcaattgttggcgaaatctcaccattgccttcttttccaggacatactattgaggatgtcaaaaatgcaattggggtcctcatcggaggacttgaatggaatgataacacagttcgagtctctaaaaatctacagagattcgcttggagaagcagtaatgagaatgggggacctccacttactccaaaacagaaacggaaaatggcgagaacagctaggtcaaaagtttgaagagataagatggctgattgaagaagtgagacacagactaaaaacaactgaaaatagctttgaacaaataacattcatgcaagcattacaactgctgtttgaagtggaacaggagataagaactttctcatttcagcttatttaatgataaa

SEQ ID NO: 66 - protein, 566 aa, Influenza virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNDESFNWTGVTQNGTSSSCKRRSNNSFFSRLNWLTHLKFKYPALNVTMPNNEKFDKLYIWGVHHPVTDNDQIFLYAQASGRITVSTKRSQQTVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 67 - protein, 469 aa, Influenza virus
MNPNQKIITIGSVSLTISTICFFMQIAILITTVTLHFKQYEFNSPPNNQVMLCEPTIIERNITEIVYLTNTTIEKEICPKLAEYRNWSKPQCNITGFAPFSKDNSIRLSAGGDIWVTREPYVSCDPDKCYQFALGQGTTLNNVHSNDTVHDRTPYRTLLMNELGVPFHLGTKQVCIAWSSSSCHDGKAWLHVCVTGDDKNATASFIYNGRLVDSIVSWSKEILRTQESECVCINGTCTVVMTDGSASGKADTKILFIEEGKIVHTSTLSGSAQHVEECSCYPRYLGVRCVCRDNWKGSNRPIVDINIKDYSIVSSYVCSGLVGDTPRKNDSSSSSHCLDPNNEEGGHGVKGWAFDDGNDVWMGRTISEKLRSGYETFKVIEGWSNPNSKLQINRQVIVDRGNRSGYSGIFSVEGKSCINRCFYVELIRGRKEETEVLWTSNSIVVFCGTSGTYGTGSWPDGADINLMPI

SEQ ID NO: 68 - protein, 716 aa, Influenza virus
MEDFVRQCFNPMIVELAEKAMKEYGEDPKIETNKFAAICTHLEVCFMYSDFHFIDERGESIIVESGDPNALLKHRFEIIEGRDRIMAWTVINSICNTTGVEKPKFLPDLYDYKENRFIEIGVTRREVHIYYLEKANKIKSEKTHIHIFSFTGEEMATKADYTLDEESRARIKTRLFTIRQEMASKSLWDSFRQSERGEETIEEKFEITGTMRKLADQSLPPNFPSLENFRAYVDGFEPNGCIEGKLSQMSKEVNAKIEPFLRTTPRPLRLPDGPLCHQRSKFLLMDALKLSIEDPSHEGEGIPLYDAIKCMKTFFGWKEPNIVKPHEKGINPNYLMAWKQVLAELQDIENEEKIPRTKNMKRTSQLKWALGENMAPEKVDFDDCKDVGDLKQYDSDEPEPRSLASWVQNEFNKACELTDSSWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRTAIGQVSRPMFLYVRTNGTSKIKMKWGMEMRRCLLQSLQQIESMIEAESSVKEKDMTKEFFENKSETWPIGESPRGVEEGSIGKVCRTLLAKSVFNSLYASPQLEGFSAESRKLLLIVQALRDNLEPGTFDLGGLYEAIEECLINDPWVLLNASWFNSFLTHALK

SEQ ID NO: 69 - protein, 252 aa, Influenza virus
MSLLTEVETYVLSIVPSGPLKAEIAQRLENVFAGKNTDLEALMEWLKTRPILSPLTKGILGFVFTLTVPSERGLQRRRFVQNALNGNGDPNNMDKAVKLYRKLKREITFHGAKEIALSYSAGALASCMGLIYNRMGAVTTESAFGLICATCEQIADSQHKSHRQMVTTTNPLIRHENRMVLASTTAKAMEQMAGSSEQAAEAMEVASQARQMVQAMRAIGTHPSSSTGLKNDLLENLQAYQKRMGVQMQRFK

SEQ ID NO: 70 - protein, 566 aa, Influenza virus
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 71 - DNA, 2305 bp, Influenza virus
agcagaagcggtgcgtttgatttgtcataatggatacttttattacaagaaacttccagactacaataatacaaaaggccaaaaacacaatggcagaatttagtgaagatcctgaattgcaaccagcaatgctattcaatatctgcgtccatctagaggtttgctatgtaataagtgacatgaattttcttgacgaagaaggaaaagcatatacagcattagaaggacaagggaaagaacaaaacttgagaccacaatatgaagtaattgagggaatgccaagaaccatagcatggatggtccagagatccttagctcaagagcatggaatagagactcccaagtatctggctgatttgtttgattataaaaccaaaagatttatagaagttggaataacaaagggattggctgatgattacttttggaaaaagaaagaaaagttgggaaatagcatggaactgatgatattcagctacaatcaagactactcgttaagtaatgaatcctcattggatgaggaagggaaagggagagtgctaagcagactcacagaacttcaggctgaattaagtctgaaaaatttatggcaagttctcataggagaagaagatgttgaaaagggaattgattttaaacttggacaaacaatatctagactaagggatatatctgttccagctggtttctccaattttgaaggaatgaggagctacatagacaatatagacccaaaaggagcaatagagagaaatctagcaaggatgtctcccttagtatcagtcacacctaaaaagttaacatgggaggacctaagaccaatagggcctcacatttacgaccatgagctaccagaagttccatataatgcctttcttctaatgtctgatgaactgggattggccaatatgactgagggaaagtccaaaaaaccgaagacattagccaaagaatgtctagaaaagtactcaacactacgggatcaaactgacccaatattaataatgaaaagcgaaaaagctaacgaaaatttcctatggaagctttggagagactgtgtaaatacaataagtaatgaggaaacgagtaacgagttacagaaaaccaattatgccaaatgggccacaggggatggattaacataccagaaaataatgaaagaagtagcaatagatgacgaaacaatgtgccaagaagagcctaaaatccctaacaaatgtagagtggctgcttgggttcaaacagagatgaatctattgagcactctgacaagtaaaagagctctggacctaccagaaatagggccagacatagcacccgtggagcatgtaggaagtgaaagaaggaaatactttgttaatgaaatcaactactgtaaggcctctacagttatgatgaagtatgtgctttttcacacttcattgttgaatgaaagcaatgccagcatgggaaaatacaaagtaataccaataaccaatagagtagtaaatgaaaaaggagaaagtttcgacatgctttacggtctggcggttaaaggacaatctcatctgaggggagatactgatgttgtaacagttgtaactttcgaatttagtagtacagatccaagagtggactcaggaaagtggccaaaatatactgtgtttaggattggctccctatttgtgagtgggagggaaaaatctgtgtacttgtattgccgagtgaatggcacaaataagatccaaatgaaatggggaatggaagctagaagatgtttgcttcaatcaatgcaacaaatggaggcaattgttgaacaggaatcatcaatacaaggatatgacatgaccaaagcctgtttcaagggagacagagtaaatagccccaaaactttcagtattggaactcaagaaggaaaactagtaaaaggatcctttggaaaagcactaagagtaatatttactaaatgcttgatgcactatgtatttggaaatgcccaattggaggggtttagtgccgagtctaggagacttctactgttgattcaagcattaaaggacagaaagggcccttgggtgttcgacttagagggaatgtattctggaatagaagaatgtattagcaacaacccttgggtaatacagagtgtatactggttcaatgaatggttgggctttgaaaaggaggggaataaagtgttggaatcagtggatgaaataatggatgaataaaaggaaatggtactcaatttggtactattttgttcattatgtatctaaacatccaataaaaagaaccaagaatcaaaaatgcacgtgtttctact

SEQ ID NO: 72 - DNA, 2369 bp, Influenza virus
agcagaagcggagcctttaagatgaatataaatccttattttctcttcatagatgtgcccgtacaggcagcaatttcaacaacattcccatacactggtgttcccccttattcccatggaacaggaacaggctacacaatagacaccgtgatcagaacgcatgagtactcaaacaaggggaaacagtacatttctgatgttacaggatgcacaatggtagatccaacaaatggaccattacccgaagataatgagccgagtgcctatgcgcaattagattgcgttttagaggctttggatagaatggatgaagaacacccaggtctttttcaagcagcctcacagaatgctatggaggccctaatggtcacaactgtagacaaattaacccaggggagacagacttttgattggacagtatgcagaaaccaacctgctgcaacggcactgaacacaacaataacctcttttaggttgaatgatttaaatggagccgacaaaggtggattaatacctttttgccaggatatcattgattcattagaccgacctgaaatgactttcttctcagtaaagaatataaagaaaaaattgcctgccaaaaacagaaagggtttcctcataaagaggataccaatgaaggtaaaagacaaaataaccaaagtggaatacatcaaaagagcattatcattaaacacaatgacaaaagacgctgaaagaggcaaactgaaaagaagagcgattgccactgctggaatacaaatcagagggtttgtattagtagttgaaaacttggctaaaaatatatgtgaaaatctagaacaaagtggtttaccagtaggtggaaacgagaagaaagccaaactgtcaaacgcagtggccaaaatgctcagtaactgcccaccaggagggattagcatgacagtaacaggagacaatacaaaatggaatgaatgtttaaacccaagaatctttttggctatgactgaaagaataaccagagacagcccagtttggttcagggatttttgtagtatagcaccggtcctgttctccaataagatagcaagattggggaaagggtttatgataacaagcaaaacaaaaagactgaaggctcaaataccttgtcctgatctgtttagtataccgttagaaagatataatgaagaaacaagggcaaaattgaaaaagctaaaaccattcttcaatgaagaaggaactgcatctttgtcgcctgggatgatgatgggaatgtttaatatgctatctaccgtgttgggagtagctgcactaggtatcaagaacattggaaacaaagaatacttatgggatggactgcaatcttctgatgattttgctctgtttgttaatgcaaaggatgaagaaacatgtatggaaggaataaacgacttttaccgaacatgtaaattattgggagtaaacatgagcaaaaagaaaagttactgtaatgagactggaatgtttgaatttacaagcatgttctacagagatggatttgtatctaattttgcaatggaactcccttcgtttggggttgctggagtaaatgaatcagcagatatggcaataggaatgacaataataaagaacaacatgatcaacaatggaatgggtccggcaacagcacaaacagccatacagttattcatagctgattatagatacacctacaaatgccacaggggagattccaaagtagaaggaaagagaatgaaaatcataaaggagttatgggaaaacactaaaggaagagatggtctattagtagcagatggtgggcccaacatttacaatttgagaaacctgcatatcccagaaatagtattaaagtataatctaatggaccctgaatacaaagggcggttacttcatcctcaaaatccctttgtgggacatttgtctattgagggcatcaaagaggcagacataaccccagcacatggtccagtaaagaaaatggactacgatgcggtgtctggaactcatagttggagaaccaaaagaaacagatctatactaaacactgatcagaggaacatgattcttgaggaacaatgctacgctaaatgttgcaacctatttgaggcctgttttaacagtgcatcatacaggaagccagtgggtcaacatagcatgcttgaggctatggcccacagattaagaatggatgcacgattagattatgaatcagggagaatgtcaaaggatgattttgagaaagcaatggctcaccttggtgagattgggtacatataagcttcgaagatgtttatggggttattggtcatcattgaatacatgcgatacacaaatgattaaaatgaaaaaaggctcgtgtttctact

SEQ ID NO: 73 - DNA, 2396 bp, Influenza virus
agcagaagcggagcgttttcaagatgacattggccaaaattgaattgttaaaacaactgctaagggacaatgaagccaaaacagttttgaagcaaacaacggtagaccaatataacataataagaaaattcaatacatcaaggattgaaaagaatccttcactaaggatgaagtgggccatgtgttctaattttcccttggctctaaccaagggcgatatggcaaacagaatccccttggaatacaaagggatacaacttaaaacaaatgctgaagacataggaaccaaaggccaaatgtgctcaatagcagcagttacttggtggaatacatatggaccaataggagatactgaaggtttcgaaagggtctacgaaagcttttttctcagaaaaatgagacttgacaacgccacttggggccgaataacttttggcccagttgaaagagtgagaaaaagggtactgctaaaccctctcaccaaggaaatgcctccggatgaggcgagcaatgtgataatggaaatattgttccctaaagaagcaggaataccaagagaatccacttggatacatagggaactgataaaagaaaaaagagaaaaattgaaaggaacaatgataactccaatcgtactggcatacatgcttgaaagagaactggttgctcgaagaagattcttgccagtggcaggagcaacatcagctgagttcatagaaatgctacactgcttacaaggtgaaaattggagacaaatatatcacccaggagggaataaattaactgagtccaggtctcaatcaatgatagtagcttgtagaaaaataatcagaagatcaatagtcgcttcaaacccactggagctagctgtagaaattgcaaacaagactgtgatagatactgaacctttaaagtcatgtctggcagccatagacggaggtgatgtagcttgtgacataataagagctgcattaggactaaagatcagacaaagacaaagatttggacggcttgagctaaaaagaatatcaggaagaggattcaaaaatgatgaagaaatattaatagggaacggaacaatacagaagattggaatatgggacggggaagaggagttccatgtaagatgtggtgaatgcaggggaatattaaaaaagagtaaaatgaaactggaaaaactactgataaattcagccaaaaaggaggatatgagagatttaataatcttatgcatggtattttctcaagacactaggatgttccaaggagtgagaggagaaataaattttcttaatcgagcaggccaacttttatctccaatgtaccaactccaacgatattttttgaatagaagcaacgacctttttgatcaatgggggtatgaggaatcacccaaagcaagtgaactacatgggataaatgaatcaatgaatgcatctgactatacattgaaagggattgtagtgacaagaaatgtaattgacgactttagctctattgaaacagaaaaagtatccataacaaaaaatcttagtttaataaaaaggactggggaagtcataatgggagctaatgacgtgagtgaattagaatcacaagcacagctgatgataacatatgatacacctaaaatgtgggaaatgggaacaaccaaagaactggtgcaaaacacttatcaatgggtgctaaaaaacttggtgacactgaaggctcagtttcttctaggaaaagaggacatgttccaatgggatgcatttgaagcatttgagagcataattcctcagaagatggctggtcagtacagtggatttgcaagagcagtgctcaaacaaatgagagaccaggaggttatgaaaactgaccagttcataaagttgttgcctttttgtttctcaccaccaaaattaaggagcaatggggagccttatcaattcttaaaacttgtgttgaaaggaggaggggaaaatttcatcgaagtaaggaaagggtcccctctattttcctataatccacaaacagaagtcctaactatatgcggcagaatgatgtcattaaaagggaaaattgaagatgaagaaaggaatagatcaatgggtaatgcagtattagcaggctttctcgttagtggcaagtatgacccagatcttggagatttcaaaactattgaagaacttgaaaagctgaaaccgggggaaaaggcaaacatcttactttatcaaggaaaaccagttaaagtagttaaaaggaaaaggtatagtgctttgtccaatgacatttcacaaggaattaagagacaaagaatgacagttgagtctatggggtgggccttgagctaatataaatttatccattaattcaatgaacgcaattgagtgaaaaatgctcgtgtttctact

SEQ ID NO: 74 - DNA, 1844 bp, Influenza virus
agcagaagcacagcattttcttgtgaacttcaagcaccagtaaaagaactgaaaatcaaaatgtccaacatggatattgacggtataaacactgggacaattgacaaaacaccggaagaaataacttctggaaccagtgggacaaccagaccaatcattagaccagcaacccttgccccaccaagcaacaaacgaacccgtaacccatccccggaaagagcaaccacaagcagtgaagatgatgtcggaaggaaaacccaaaagaaacagaccccgacagagataaagaagagcgtctacaacatggtggtgaaactgggcgaattctataaccagatgatggtcaaagctggactcaatgatgacatggagagaaatctaatccaaaatgcgcatgccgtggaaagaattctattggctgccactgatgacaagaaaaccgagttccagaagaaaaagaatgccagagatgtcaaagaagggaaagaagaaatagatcacaacaaaacaggaggcaccttttacaagatggtaagagatgataaaaccatctacttcagccctataagaattacctttttaaaagaagaggtgaaaacaatgtacaaaaccaccatggggagtgatggcttcagtggactaaatcacataatgattgggcattcacagatgaatgatgtctgtttccaaagatcaaaggcactaaaaagagttggacttgatccttcattaatcagtacctttgcgggaagcacagtccccagaagatcaggtgcgactggtgttgcaatcaaaggaggtggaaccttagtggctgaagccattcgatttataggaagagcaatggcagacagagggctattgagagacatcaaagccaagactgcctatgaaaagattcttctgaatctaaaaaacaaatgctctgcgccccaacaaaaggctctagttgatcaagtgatcggaagcagaaatccggggattgcagacattgaagatctaaccctgcttgctcgtagtatggtcgttgttaggccctctgtggcaagcaaagtggtgcttcccataagcatttacgccaaaatacctcaactagggttcaatgttgaagagtactctatggttgggtacgaagccatggctctttacaatatggcaacacctgtgtccatattaagaatgggagatgatgcaaaagataaatcgcaattattcttcatgtcttgcttcggagctgcctatgaagacctgagagttttgtctgcattaacaggcacagaattcaagcctagatcagcattaaaatgcaagggtttccatgttccagcaaaggaacaggtagaaggaatgggagcagctctgatgtccatcaagctccagttttgggctccgatgaccagatctggggggaacgaagtaggtggagacggagggtctggccaaataagctgcagcccagtgtttgcagtggaaagacctattgctctaagcaagcaagctgtaagaagaatgctgtcaatgaatattgagggacgtgatgcagatgtcaaaggaaatctactcaagatgatgaatgactcaatggctaagaaaaccagtggaaatgctttcattgggaagaaaatgtttcaaatatcagacaaaaacaaaaccaatcccattgaaattccaattaagcagaccatccccaatttcttctttgggagggacacagcagaggattatgatgacctcgattattaaggcaacaaaatagacactatgactgtgattgtttcaatacgtttggaatgtgggtgtttattcttattaaaataaatataaaaaatgctgttgtttctact

SEQ ID NO: 75 - DNA, 1189 bp, Influenza virus
agcagaagcacgcactttcttaaaatgtcgctgtttggagacacaattgcctacctgctttcattgacagaagatggagaaggcaaagcagaactagcagaaaaattacactgttggtttggtgggaaagaatttgacctagactctgccttggaatggataaaaaacaaaagatgcttaactgatatacaaaaagcactaattggtgcctctatatgctttttaaaacccaaagaccaggaaagaaaaagaagattcatcacagagcccttatcaggaatgggaacaacagcaacaaaaaagaaaggcctgattctggctgagagaaaaatgagaagatgtgtgagctttcatgaagcatttgaaatagcagaaggccatgaaagctcagcgctactatactgtctcatggtcatgtacctgaatcctggaaattattcaatgcaagtaaaactaggaacgctctgtgctttatgcgagaaacaagcatcacattcacacagggctcatagcagagcagcgagatcttcagtgcctggagtgagacgagaaatgcagatggtctcagctatgaacacagcaaaaacaatgaatggaatgggaaaaggagaagacgtccaaaagctggcagaagagttgcaaagcaacattggagtgctgagatctcttggggcaagccaaaagaatggggaagggattgcaaaggatgtaatggaagtgctaaagcagagctccatgggaaattcagctcttgtgaagaaatatctataatgctcgaaccatttcagattcttacaatttgttcttttatcttatcagctctccatttcatggcttggacaatagggcatttgaatcaaataaaaagaggaataaacatgaaaatacgaataaaaggtccaaacaaagagacaataaacagagaggtatcaattttgagacacagttaccaaaaagaaatccaggccaaagaaacaatgaaggaagtactctctgacaacatggaggtattgaatgaccacataataattgaggggctttctgccgaagagataataaaaatgggtgaaacagttttggagatagaagaattgcattaaattcaattttactgtatttcttactatgcatttaagcaaattgtaatcaatgtcagcaaataaactggaaaaagtgcgttgtttctact

SEQ ID NO: 76 - DNA, 1101 bp, Influenza virus
agcagaagcagaggatttgtttagtcactggcaaacagggaaaaatggcgaacaacaacatgaccacaacacaaattgaggtgggtccgggagcaaccaatgccaccataaactttgaagcaggaattctagagtgctatgaaaggctttcatggcaaagagcccttgactaccctggtcaagaccgcctaaacagactaaagagaaaattagagtcaagaataaagactcacaacaaaagtgagcctgaaagtaaaaggatgtcccttgaagagagaaaagcaattggagtaaaaatgatgaaagtactcctatttatgaatccgtctgctggaattgaagggtttgagccatactgtatgaaaagttcctcaaatagcaactgtacgaaatacaattggactgattacccttcaacaccagagaggtgccttgatgacatagaggaagaaccagaggatgttgatggcccaactgaaatagtattaagggacatgaacaacaaagatgcaaggcaaaagataaaggaggaagtaaacactcagaaagaagggaagttccgtttgacaataaaaagggatatgcgtaatgtattgtccttgagagtgttggtaaacggaacattcctcaaacaccccaatggacacaagtccttatcaactctgcatagattgaatgcatatgaccagagtggaaggcttgttgctaaacttgttgccactgatgatcttacagtggaggatgaagaagatggccatcggatcctcaactcactcttcgagcgtcttaatgaaggacattcaaagccaattcgagcagctgaaactgcggtgggagtcttatcccaatttggtcaagagcaccgattatcaccagaagagggagacaattagactggtcacggaagaactttatcttttaagtaaaagaattgatgataacatactattccacaaaacagtaatagctaacagctccataatagctgacatggttgtatcattatcattattagaaacattgtatgaaatgaaggatgtggttgaagtgtacagcaggcagtgcttgtgaatttaaaataaaaatcctcttgttactact

SEQ ID NO: 77 - DNA, 2305 bp, Influenza virus
agcagaagcggtgcgtttgatttgccataatggatacttttattacaagaaacttccagactacaataatacaaaaggccaaaaacacaatggcagaatttagtgaagatcctgaattacaaccagcaatgctattcaacatctgcgtccatctagaggtttgctatgtaataagtgacatgaattttcttgacgaagaaggaaaatcatatacagcattagaaggacaaggaaaagaacaaaacttgagaccacaatatgaagtaattgagggaatgccaagaaccatagcatggatggtccaaagatccttagctcaagagcatggaatagagactccaaagtatctggctgatttgtttgattataaaaccaagagatttatagaagttggaataacaaaaggattggctgatgattacttttggaaaaagaaagaaaagctgggaaatagcatggaactgatgatattcagctacaatcaagactattcgttaagtaatgaatcctcattggatgaggaagggaaagggagagtgctaagcagactcacagaacttcaggctgaattaagtctgaaaaacctatggcaagttctcataggagaagaagatgttgaaaagggaattgactttaaacttggacaaacaatatctagactaagggatatatctgttccagctggtttctccaattttgaaggaatgaggagctacatagacaatatagatcctaaaggagcaatagaaagaaatctagcaaggatgtctcccttagtatcagccacacctaaaaagttgaaatgggaggacctaagaccaatagggcctcacatttacaaccatgagttaccagaagttccatataatgcctttcttctaatgtctgatgaattggggctggccaatatgactgagggaaagtccaaaaaaccgaagacattagccaaagaatgtctagaaaagtactcaacactacgggatcaaactgacccaatattaataatgaaaagcgaaaaagctaacgaaaatttcctatggaagctgtggagggactgtgtaaatacaataagtaatgaggaaatgagtaacgagttacagaaaaccaattatgccaagtgggccacaggagatggattaacataccagaaaataatgaaagaagtagcaatagatgacgaaacaatgtgccaagaagagcctaaaatccctaacaaatgtagagtggctgcttgggttcaaacagagatgaatttattgagcactctgacaagtaaaagagctctggacctaccagaaatagggccagacgtagcacccgtggagcatgtagggagtgaaagaaggaaatactttgttaatgaaatcaactgctgtaaggcctctacagttatgatgaagtatgtgctttttcacacttcattattgaatgaaagcaatgccagcatgggaaaatataaagtaataccaataaccaatagagtagtaaatgaaaaaggagaaagtttcgacatgctttatggtctggcggttaaaggacaatctcatctgaggggagatactgatgttgtaacagttgtgactttcgaatttagtggtacagatcccagagtggactcaggaaagtggccaaaatatactgtgtttaggattggctccctatttgtgagtgggagggaaaaatctgtgtacctatattgccgagtgaatggcacaaataagatccaaatgaaatggggaatggaagctagaagatgtctgcttcaatcaatgcaacaaatggaagcaattgttgaacaagaatcatcgatacaaggatatgacatgaccaaagcttgtttcaagggagacagagtaaatagccccaaaacttttagtattgggactcaagaaggaaaactagtaaaaggatcctttgggaaagcactaagagtaatatttaccaaatgtttgatgcactatgtatttggaaatgcccaattggaggggtttagtgccgagtctaggagacttctactgttaattcaagcactaaaggacagaaagggcccttgggtgttcgacttagagggaatgtattctggaatagaagaatgtattagtaacaacccttgggtaatacagagtgcatactggttcaatgaatggttgggctttgaaaaggaggggagtaaagtattagaatcagtagatgaaataatgaatgaatgaaaaaacatagtactcaatttggtactattttgttcattatgtatctaaacatccaataaaaagaatcgagaatcaaaaatgcacgtgtttctact

SEQ ID NO: 78 - DNA, 2369 bp, Influenza virus
agcagaagcggagcctttaagatgaatataaatccttattttctcttcatagatgtacccatacaggcagcaatttcaacaacattcccatacaccggtgttcccccttactcccatggaacgggaacaggccacacaatagacaccgtgatcagaacacatgagtactcgaacaagggaaaacagtatgtttctgacatcacaggatgtacaatggtagatccaacaaatgggccattacccgaagacaatgagccgagtgcctatgcacaattagattgcgttctggaggctttggatagaatggatgaagaacatccaggtttgtttcaagcagcctcacagaatgccatggaggcactaatggtcacaactgtagacaaattaacccaggggagacagacttttgattggacagtatgcagaaaccagcctgctgcaacggcactaaacacaacaataacctcctttaggttgaatgatttgaatggagctgacaagggtggattggtacccttttgccaagatatcattgattcattggacaaacctgaaatgactttcttctcagtaaagaatataaagaaaaaattgcctgctaaaaacagaaagggtttcctcataaagagaataccaatgaaagtaaaagacaggataaccagagtggaatacatcaaaagagcattatcattaaacacaatgacaaaagatgctgaaaggggcaaactaaaaagaagagcgattgcaaccgctggaatacaaatcagagggtttgtattagtagttgaaaacttggctaaaaatatctgtgaaaatctagaacaaagtggtttgcccgtaggtggaaatgaaaagaaggccaaactgtcaaatgcagtggccaaaatgctcagtaactgcccaccaggagggatcagcatgacagtaacaggagacaatactaaatggaatgaatgcttaaatccaagaatctttttggctatgactgaaaggataacaagagacagcccaatttggttccgggatttttgtagtatagcaccggtcttgttctccaataaaatagccagattgggaaaaggatttatgataacaagcaaaacaaaaagactgaaggctcaaataccttgtccagatctgtttagcataccattagaaagatataatgaagaaacaagggcaaaattaaaaaagctgaaaccattcttcaatgaagaaggaacggcatctttgtcgcctgggatgatgatgggaatgtttaatatgctatctaccgtgttgggagtagccgcactaggtatcaaaaacattggaaacaaagaatatttatgggatggactgcaatcttctgatgattttgctctgtttgttaatgcaaaagatgaagagacatgtatggaaggaataaacgacttttaccgaacatgtaaattattgggaataaacatgagcaaaaagaaaagttactgtaatgaaactggaatgtttgaatttacaagcatgttctatagagatggatttgtatctaattttgcaatggaaattccttcatttggagttgctggagtaaatgaatcagcagatatggcaataggaatgacaataataaagaacaatatgatcaacaatgggatgggtccagcaacagcacaaacagccatacaattattcatagctgattataggtacacctacaaatgccacaggggagattccaaagtggaaggaaaaagaatgaaaattataaaggagctatgggaaaacactaaaggaagagatggtctgttagtggcagatggtgggcccaacatttacaatttgagaaacttacatatcccagaaatagtattgaagtacaacctaatggaccctgaatacaaagggcggttacttcatcctcaaaatccatttgtaggacatttatctattgagggcatcaaagaagcagatataaccccagcacatggtcccgtaaagaaaatggattatgatgcagtatctggaactcatagttggagaaccaaaaggaacagatctatactaaatactgaccagaggaacatgattcttgaggaacaatgctacgctaagtgttgcaacctttttgaggcctgttttaatagtgcatcatacaggaaaccagtaggtcagcacagcatgcttgaggctatggcccacagattaagagtggatgcacgactagattatgaatcaggaagaatgtcaaaggatgattttgagaaagcaatggctcaccttggtgagattgggtacatataagctccgaagatgtctatggggttattggtcatcattgaatacatgtgataaacaaatgattaaaatgaaaaaaggctcgtgtttctact

SEQ ID NO: 79 - DNA, 2396 bp, Influenza virus
agcagaagcggagcgttttcaagatgacattggctaaaattgaattgttaaaacaactgttaagggacaatgaagccaaaacagtattgaaacaaacaacggtagaccaatataacataataagaaaattcaatacatcaagaattgaaaagaacccttcattgaggatgaagtgggcaatgtgttctaattttcccttggctctgaccaagggtgatatggcaaacagaatccccttggaatacaagggaatacaacttaaaacaaatgctgaagacataggaactaaaggccaaatgtgctcaatagcagcagttacctggtggaatacatatggaccaataggagatactgaaggtttcgaaaaggtctacgaaagcttttttctcagaaagatgagacttgacaatgccacttggggccgaataacttttggcccagttgaaagagtaagaaaaagggtactgctaaaccctctcaccaaggaaatgcctccagatgaagcaagtaatgtgataatggaaatattgttccctaaggaagcaggaataccaagagaatctacttggatacatagggaactgataaaagaaaaaagagaaaaattgaaaggaacaatgataactcccattgtactggcatacatgcttgagagagaattggttgccagaagaaggttcctgccggtggcaggagcaacatcagctgagttcatagaaatgctacactgcttacaaggtgaaaattggagacaaatatatcacccaggaggaaataaactaactgaatctaggtctcaatcgatgattgtagcttgtagaaagataatcagaagatcaatagtcgcatcaaacccattagagctagctgtagaaattgcaaacaagactgtgatagatactgaacctttaaaatcatgtctgacagccatagacggaggtgatgtagcctgtgacataataagagctgcattaggactaaagatcagacaaagacaaagatttggacgacttgaactaaagagaatatcaggaagaggattcaaaaatgatgaagaaatattaatcgggaacggaacaatacagaagattggaatatgggacggagaagaggagttccatgtaagatgtggtgaatgcaggggaatattaaaaaagagcaaaatgagaatggaaaaactactaataaattcagctaaaaaggaagacatgaaagatttaataatcttgtgcatggtattttctcaagacactaggatgttccaaggagtgagaggagaaataaattttcttaatagagcaggccaacttttatctccaatgtaccaactccaaagatattttttgaatagaagcaacgatctctttgatcaatgggggtatgaggaatcacccaaagcaagtgagctacatggaataaatgaattaatgaatgcatctgactacactttgaaaggggttgtagtaacaaaaaatgtaattgatgattttagttctactgaaacagaaaaagtatctataacaaaaaatcttagtttaataaaaaggactggggaagtcataatgggggctaatgacgtaagtgaattagaatcacaagctcagctaatgataacatatgatacacctaagatgtgggagatgggaacaaccaaagaactggtgcaaaacacctaccaatgggtgctgaaaaatttggtaacactgaaggctcagtttcttctaggaaaagaagacatgttccaatgggatgcatttgaagcatttgaaagcataatcccccagaagatggctggccagtacagtggatttgcaagagcagtgctcaaacaaatgagagaccaagaggttatgaaaactgaccagttcataaagttgttgcccttttgtttctcaccaccaaaattaaggagaaatggggagccttatcagttcttgaggcttgtattgaagggaggaggagaaaatttcatcgaagtaaggaaagggtcccctctattctcttacaatccacaaacagaagtcctaactatatgcggcagaatgatgtcattaaaagggaaaattgaagatgaagaaaggaatagatcaatggggaatgcagtattagcgggctttctcgttagtggcaagtatgacccagatcttggagatttcaaaactattgaagaacttgaaaagctgaaaccgggggagaaagcaaacatcttactttatcaaggaaagcccgttaaagtagttaaaaggaaaagatatagtgctttatccaatgacatttcacaaggaattaagagacaaagaatgacagttgagtccatggggtgggccttgagctaatataaatttatccattaattcaataaacacaattgagtgaaaaatgctcgtgtttctact

SEQ ID NO: 80 - DNA, 1844 bp, Influenza virus
agcagaagcacagcattttcttattaacttcaagtaccaacaaaagaactgaaaatcaaaatgtccaacatggatattgacggtatcaacactgggacaattgacaaaacaccggaagaaataacttctggaaccagtgggacaaccagaccaatcatcagaccagcaacccttgccccaccaagcaacaaacgaacccggaacccatccccggaaagagcaaccacaagcagtgaagctgatgtcggaaggaaaacccaaaagaaacagaccccgacagagataaagaagagcgtctacaatatggtagtgaaactgggtgaattctataaccagatgatggtcaaagctggactcaacgatgacatggagagaaacctaatccaaaatgcgcatgctgtggaaagaattctattggctgccactgatgacaagaaaactgaattccagaggaaaaagaatgccagagatgtcaaagaaggaaaagaagaaatagaccacaacaaaacaggaggcaccttttacaagatggtaagagatgataaaaccatctacttcagccctataagaattacctttttaaaagaagaggtgaaaacaatgtacaaaaccaccatggggagtgatggcttcagtggactaaatcacataatgattgggcattcacagatgaatgatgtctgtttccaaagatcaaaggccctaaaaagagttggacttgacccttcattaatcagtacctttgcaggaagcacactccccagaagatcaggtgcaactggtgttgcaatcaaaggaggtggaactttagtggctgaagccattcgatttataggaagagcaatggcagacagagggctattgagagacatcaaagccaagactgcctatgaaaagattcttctgaatctaaaaaacaaatgctctgcgccccaacaaaaggctctagttgatcaagtgatcggaagtagaaatccagggattgcagacattgaagacctaaccctgcttgctcgtagtatggtcgttgttaggccctctgtggcgagcaaagtagtgcttcccataagcatttatgctaaaatacctcaactagggttcaatgttgaagaatactctatggttgggtatgaagccatggctctctacaatatggcaacacctgtttccatattaagaatgggagatgatgcaaaagataaatcgcaattattcttcatgtcttgcttcggagctgcctatgaagacctgagagttttgtctgcattaacaggcatagaattcaagcctagatcagcattaaaatgcaagggtttccatgttccagcaaaggaacaggtggaaggaatgggggcagctctgatgtccatcaagctccagttttgggctccaatgaccagatctggagggaacgaagtaggtggagacggagggtctggccaaataagttgcagcccagtgtttgcagtagaaagacctattgctctaagcaagcaagctgtaagaagaatgctttcaatgaatattgagggacgtgatgcagatgtcaaaggaaatctactcaagatgatgaatgactcaatggctaagaaaaccaatggaaatgctttcattgggaagaaaatgtttcaaatatcagacaaaaacaaaaccaatcccgttgaaattccaattaagcagaccatccccaatttcttctttgggagggacacagcagaggattatgatgacctcgattattaaagcaacaaaatagacactatgactgtgattgtttcaatacgtttggaatgtgggtgtttactcttattgaaataaatataaaaaatgctgttgtttctact

SEQ ID NO: 81 - DNA, 1190 bp, Influenza virus
agcagaagcacgcactttcttaaaatgtcgctgtttggagacacaattgcctacctgctttcattgacagaagatggagaaggcaaagcagaactagcagaaaaattacactgttggttcggtgggaaagaatttgacctagactctgccttggaatggataaaaaacaaaagatgcttaactgatatacagaaagcactaattggtgcctctatctgctttttaaaaccaaaagaccaagaaagaaaaagaagattcatcacagagcccctatcaggaatgggaacaacagcaacaaaaaagaagggcctgattctagctgagagaaaaatgagaagatgtgtgagttttcatgaagcatttgaaatagcagaaggccatgaaagctcagcgctactatattgtctcatggtcatgtacctgaaccctggaaattattcaatgcaagtaaaactaggaacgctctgtgctttgtgcgagaaacaagcatcacattcacacagggctcatagcagagcagcaagatcttcagtgcctggagtgaggcgagaaatgcagatggtctcagctatgaacacagcaaaaacaatgaatggaatgggaaagggagaagacgtccaaaaactggcagaagagctgcaaagcaacattggagtattgagatctcttggggcaagtcaaaagaatggggaaggaattgcaaaggatgtgatggaagtgctaaagcagagctctatgggaaattcagctcttgtgaagaaatacctataatgctcgaaccatttcagattctttcaatttgttctttcatcttatcagctctccatttcatggcttggacaatagggcatttgaatcaaataaaaagaggagtaaacatgaaaatacgaataaaaaatccaaataaagagacaataaacagagaggtatcaattttgagacacagttaccaaaaagaaatccaggccaaagaaacaatgaaggaagtactctctgacaacatggaggtattgagtgaccacatagtaattgaggggctttctgctgaagagataataaaaatgggtgaaacagttttggaggtagaagaattgcattaaattcaatttttactgtatttcttgctatgcatttaagcaaattgtaatcaatgtcagcaaataaactggaaaaagtgcgttgtttctact

SEQ ID NO: 82 - DNA, 1096 bp, Influenza virus
agcagaagcagaggatttgtttagtcactggcaaacgaaaaaatggcggacaacatgaccacaacacaaattgaggtgggtccgggagcaaccaatgccaccataaactttgaagcaggaattttggagtgctatgaaaggctttcatggcaaagagcccttgactaccctggtcaagaccgcctaaacaaactaaagagaaaattggaatcaagaataaagactcacaacaaaagtgagccagaaagtaaaaggatgtctcttgaagagagaaaagctattggggtaaaaatgatgaaagtgctcctatttatgaacccatctgctggagttgaagggtttgagccatattgtatgaaaaatccctccaatagcaactgtccagactgcaattgggctgattaccctccaacaccaggaaagtaccttgatggcatagaagaagaaccggagaatgttggtgactcaactgaaatagtattaagggacatgaacaacaaagatgcaaggcaaaagataaaagaggaagtaaacactcagaaagaagggaaattccgtttgacaataaaaagggatatacgtaatgtgttgtccttgagagtgttggtaaacggaacattcatcaagcaccctaatggatacaagtccttatcaactctgcatagattgaatgcatatgaccagagtggaagacttgttgctaaacttgttgctactgatgatcttacagtggaggatgaagaagatggccatcggatcctcaactcactcttcgagcgtcttaatgaaggacattcaaagccaattcgagcagctgaaactgcggtgggagtcttatcccaatttggtcaagagcaccgattatcaccagaagagagagacaattagactggttacggaagaactttatcttttaagtaaaagaattgatgataacatattgttccacaaaacagtaatagccaacagctccataatagctgacatgattgtatcattatcattattggaaacattgtatgaaatgaaggatgtggttgaagtgtacagcaggcagtgcttgtgaatttaaaataaaaatcctcttgttactact

SEQ ID NO: 83 - protein, 726 aa, Influenza virus
MDTFITRNFQTTIIQKAKNTMAEFSEDPELQPAMLFNICVHLEVCYVISDMNFLDEEGKAYTALEGQGKEQNLRPQYEVIEGMPRTIAWMVQRSLAQEHGIETPKYLADLFDYKTKRFIEVGITKGLADDYFWKKKEKLGNSMELMIFSYNQDYSLSNESSLDEEGKGRVLSRLTELQAELSLKNLWQVLIGEEDVEKGIDFKLGQTISRLRDISVPAGFSNFEGMRSYIDNIDPKGAIERNLARMSPLVSVTPKKLTWEDLRPIGPHIYDHELPEVPYNAFLLMSDELGLANMTEGKSKKPKTLAKECLEKYSTLRDQTDPILIMKSEKANENFLWKLWRDCVNTISNEETSNELQKTNYAKWATGDGLTYQKIMKEVAIDDETMCQEEPKIPNKCRVAAWVQTEMNLLSTLTSKRALDLPEIGPDIAPVEHVGSERRKYFVNEINYCKASTVMMKYVLFHTSLLNESNASMGKYKVIPITNRVVNEKGESFDMLYGLAVKGQSHLRGDTDVVTVVTFEFSSTDPRVDSGKWPKYTVFRIGSLFVSGREKSVYLYCRVNGTNKIQMKWGMEARRCLLQSMQQMEAIVEQESSIQGYDMTKACFKGDRVNSPKTFSIGTQEGKLVKGSFGKALRVIFTKCLMHYVFGNAQLEGFSAESRRLLLLIQALKDRKGPWVFDLEGMYSGIEECISNNPWVIQSVYWFNEWLGFEKEGNKVLESVDEIMDE

SEQ ID NO: 84 - protein, 752 aa, Influenza virus
MNINPYFLFIDVPVQAAISTTFPYTGVPPYSHGTGTGYTIDTVIRTHEYSNKGKQYISDVTGCTMVDPTNGPLPEDNEPSAYAQLDCVLEALDRMDEEHPGLFQAASQNAMEALMVTTVDKLTQGRQTFDWTVCRNQPAATALNTTITSFRLNDLNGADKGGLIPFCQDIIDSLDRPEMTFFSVKNIKKKLPAKNRKGFLIKRIPMKVKDKITKVEYIKRALSLNTMTKDAERGKLKRRAIATAGIQIRGFVLVVENLAKNICENLEQSGLPVGGNEKKAKLSNAVAKMLSNCPPGGISMTVTGDNTKWNECLNPRIFLAMTERITRDSPVWFRDFCSIAPVLFSNKIARLGKGFMITSKTKRLKAQIPCPDLFSIPLERYNEETRAKLKKLKPFFNEEGTASLSPGMMMGMFNMLSTVLGVAALGIKNIGNKEYLWDGLQSSDDFALFVNAKDEETCMEGINDFYRTCKLLGVNMSKKKSYCNETGMFEFTSMFYRDGFVSNFAMELPSFGVAGVNESADMAIGMTIIKNNMINNGMGPATAQTAIQLFIADYRYTYKCHRGDSKVEGKRMKIIKELWENTKGRDGLLVADGGPNIYNLRNLHIPEIVLKYNLMDPEYKGRLLHPQNPFVGHLSIEGIKEADITPAHGPVKKMDYDAVSGTHSWRTKRNRSILNTDQRNMILEEQCYAKCCNLFEACFNSASYRKPVGQHSMLEAMAHRLRMDARLDYESGRMSKDDFEKAMAHLGEIGYI

SEQ ID NO: 85 - protein, 770 aa, Influenza virus
MTLAKIELLKQLLRDNEAKTVLKQTTVDQYNIIRKFNTSRIEKNPSLRMKWAMCSNFPLALTKGDMANRIPLEYKGIQLKTNAEDIGTKGQMCSIAAVTWWNTYGPIGDTEGFERVYESFFLRKMRLDNATWGRITFGPVERVRKRVLLNPLTKEMPPDEASNVIMEILFPKEAGIPRESTWIHRELIKEKREKLKGTMITPIVLAYMLERELVARRRFLPVAGATSAEFIEMLHCLQGENWRQIYHPGGNKLTESRSQSMIVACRKIIRRSIVASNPLELAVEIANKTVIDTEPLKSCLAAIDGGDVACDIIRAALGLKIRQRQRFGRLELKRISGRGFKNDEEILIGNGTIQKIGIWDGEEEFHVRCGECRGILKKSKMKLEKLLINSAKKEDMRDLIILCMVFSQDTRMFQGVRGEINFLNRAGQLLSPMYQLQRYFLNRSNDLFDQWGYEESPKASELHGINESMNASDYTLKGIVVTRNVIDDFSSIETEKVSITKNLSLIKRTGEVIMGANDVSELESQAQLMITYDTPKMWEMGTTKELVQNTYQWVLKNLVTLKAQFLLGKEDMFQWDAFEAFESIIPQKMAGQYSGFARAVLKQMRDQEVMKTDQFIKLLPFCFSPPKLRSNGEPYQFLKLVLKGGGENFIEVRKGSPLFSYNPQTEVLTICGRMMSLKGKIEDEERNRSMGNAVLAGFLVSGKYDPDLGDFKTIEELEKLKPGEKANILLYQGKPVKVVKRKRYSALSNDISQGIKRQRMTVESMGWALS

SEQ ID NO: 86 - protein, 560 aa, Influenza virus
MSNMDIDGINTGTIDKTPEEITSGTSGTTRPIIRPATLAPPSNKRTRNPSPERATTSSEDDVGRKTQKKQTPTEIKKSVYNMVVKLGEFYNQMMVKAGLNDDMERNLIQNAHAVERILLAATDDKKTEFQKKKNARDVKEGKEEIDHNKTGGTFYKMVRDDKTIYFSPIRITFLKEEVKTMYKTTMGSDGFSGLNHIMIGHSQMNDVCFQRSKALKRVGLDPSLISTFAGSTVPRRSGATGVAIKGGGTLVAEAIRFIGRAMADRGLLRDIKAKTAYEKILLNLKNKCSAPQQKALVDQVIGSRNPGIADIEDLTLLARSMVVVRPSVASKVVLPISIYAKIPQLGFNVEEYSMVGYEAMALYNMATPVSILRMGDDAKDKSQLFFMSCFGAAYEDLRVLSALTGTEFKPRSALKCKGFHVPAKEQVEGMGAALMSIKLQFWAPMTRSGGNEVGGDGGSGQISCSPVFAVERPIALSKQAVRRMLSMNIEGRDADVKGNLLKMMNDSMAKKTSGNAFIGKKMFQISDKNKTNPIEIPIKQTIPNFFFGRDTAEDYDDLDY

SEQ ID NO: 87 - protein, 248 aa, Influenza virus
MSLFGDTIAYLLSLTEDGEGKAELAEKLHCWFGGKEFDLDSALEWIKNKRCLTDIQKALIGASICFLKPKDQERKRRFITEPLSGMGTTATKKKGLILAERKMRRCVSFHEAFEIAEGHESSALLYCLMVMYLNPGNYSMQVKLGTLCALCEKQASHSHRAHSRAARSSVPGVRREMQMVSAMNTAKTMNGMGKGEDVQKLAEELQSNIGVLRSLGASQKNGEGIAKDVMEVLKQSSMGNSALVKKYL

SEQ ID NO: 88 - protein, 109 aa, Influenza virus
MLEPFQILTICSFILSALHFMAWTIGHLNQIKRGINMKIRIKGPNKETINREVSILRHSYQKEIQAKETMKEVLSDNMEVLNDHIIIEGLSAEEIIKMGETVLEIEELH

SEQ ID NO: 89 - protein, 282 aa, Influenza virus
MANNNMTTTQIEVGPGATNATINFEAGILECYERLSWQRALDYPGQDRLNRLKRKLESRIKTHNKSEPESKRMSLEERKAIGVKMMKVLLFMNPSAGIEGFEPYCMKSSSNSNCTKYNWTDYPSTPERCLDDIEEEPEDVDGPTEIVLRDMNNKDARQKIKEEVNTQKEGKFRLTIKRDMRNVLSLRVLVNGTFLKHPNGHKSLSTLHRLNAYDQSGRLVAKLVATDDLTVEDEEDGHRILNSLFERLNEGHSKPIRAAETAVGVLSQFGQEHRLSPEEGDN

SEQ ID NO: 90 - protein, 123 aa, Influenza virus
MANNNMTTTQIEWRMKKMAIGSSTHSSSVLMKDIQSQFEQLKLRWESYPNLVKSTDYHQKRETIRLVTEELYLLSKRIDDNILFHKTVIANSSIIADMVVSLSLLETLYEMKDVVEVYSRQCL

SEQ ID NO: 91 - protein, 726 aa, Influenza virus
MDTFITRNFQTTIIQKAKNTMAEFSEDPELQPAMLFNICVHLEVCYVISDMNFLDEEGKSYTALEGQGKEQNLRPQYEVIEGMPRTIAWMVQRSLAQEHGIETPKYLADLFDYKTKRFIEVGITKGLADDYFWKKKEKLGNSMELMIFSYNQDYSLSNESSLDEEGKGRVLSRLTELQAELSLKNLWQVLIGEEDVEKGIDFKLGQTISRLRDISVPAGFSNFEGMRSYIDNIDPKGAIERNLARMSPLVSATPKKLKWEDLRPIGPHIYNHELPEVPYNAFLLMSDELGLANMTEGKSKKPKTLAKECLEKYSTLRDQTDPILIMKSEKANENFLWKLWRDCVNTISNEEMSNELQKTNYAKWATGDGLTYQKIMKEVAIDDETMCQEEPKIPNKCRVAAWVQTEMNLLSTLTSKRALDLPEIGPDVAPVEHVGSERRKYFVNEINCCKASTVMMKYVLFHTSLLNESNASMGKYKVIPITNRVVNEKGESFDMLYGLAVKGQSHLRGDTDVVTVVTFEFSGTDPRVDSGKWPKYTVFRIGSLFVSGREKSVYLYCRVNGTNKIQMKWGMEARRCLLQSMQQMEAIVEQESSIQGYDMTKACFKGDRVNSPKTFSIGTQEGKLVKGSFGKALRVIFTKCLMHYVFGNAQLEGFSAESRRLLLLIQALKDRKGPWVFDLEGMYSGIEECISNNPWVIQSAYWFNEWLGFEKEGSKVLESVDEIMNE

SEQ ID NO: 92 - protein, 752 aa, Influenza virus
MNINPYFLFIDVPIQAAISTTFPYTGVPPYSHGTGTGHTIDTVIRTHEYSNKGKQYVSDITGCTMVDPTNGPLPEDNEPSAYAQLDCVLEALDRMDEEHPGLFQAASQNAMEALMVTTVDKLTQGRQTFDWTVCRNQPAATALNTTITSFRLNDLNGADKGGLVPFCQDIIDSLDKPEMTFFSVKNIKKKLPAKNRKGFLIKRIPMKVKDRITRVEYIKRALSLNTMTKDAERGKLKRRAIATAGIQIRGFVLVVENLAKNICENLEQSGLPVGGNEKKAKLSNAVAKMLSNCPPGGISMTVTGDNTKWNECLNPRIFLAMTERITRDSPIWFRDFCSIAPVLFSNKIARLGKGFMITSKTKRLKAQIPCPDLFSIPLERYNEETRAKLKKLKPFFNEEGTASLSPGMMMGMFNMLSTVLGVAALGIKNIGNKEYLWDGLQSSDDFALFVNAKDEETCMEGINDFYRTCKLLGINMSKKKSYCNETGMFEFTSMFYRDGFVSNFAMEIPSFGVAGVNESADMAIGMTIIKNNMINNGMGPATAQTAIQLFIADYRYTYKCHRGDSKVEGKRMKIIKELWENTKGRDGLLVADGGPNIYNLRNLHIPEIVLKYNLMDPEYKGRLLHPQNPFVGHLSIEGIKEADITPAHGPVKKMDYDAVSGTHSWRTKRNRSILNTDQRNMILEEQCYAKCCNLFEACFNSASYRKPVGQHSMLEAMAHRLRVDARLDYESGRMSKDDFEKAMAHLGEIGYI

SEQ ID NO: 93 - protein, 770 aa, Influenza virus
MTLAKIELLKQLLRDNEAKTVLKQTTVDQYNIIRKFNTSRIEKNPSLRMKWAMCSNFPLALTKGDMANRIPLEYKGIQLKTNAEDIGTKGQMCSIAAVTWWNTYGPIGDTEGFEKVYESFFLRKMRLDNATWGRITFGPVERVRKRVLLNPLTKEMPPDEASNVIMEILFPKEAGIPRESTWIHRELIKEKREKLKGTMITPIVLAYMLERELVARRRFLPVAGATSAEFIEMLHCLQGENWRQIYHPGGNKLTESRSQSMIVACRKIIRRSIVASNPLELAVEIANKTVIDTEPLKSCLTAIDGGDVACDIIRAALGLKIRQRQRFGRLELKRISGRGFKNDEEILIGNGTIQKIGIWDGEEEFHVRCGECRGILKKSKMRMEKLLINSAKKEDMKDLIILCMVFSQDTRMFQGVRGEINFLNRAGQLLSPMYQLQRYFLNRSNDLFDQWGYEESPKASELHGINELMNASDYTLKGVVVTKNVIDDFSSTETEKVSITKNLSLIKRTGEVIMGANDVSELESQAQLMITYDTPKMWEMGTTKELVQNTYQWVLKNLVTLKAQFLLGKEDMFQWDAFEAFESIIPQKMAGQYSGFARAVLKQMRDQEVMKTDQFIKLLPFCFSPPKLRRNGEPYQFLRLVLKGGGENFIEVRKGSPLFSYNPQTEVLTICGRMMSLKGKIEDEERNRSMGNAVLAGFLVSGKYDPDLGDFKTIEELEKLKPGEKANILLYQGKPVKVVKRKRYSALSNDISQGIKRQRMTVESMGWALS

SEQ ID NO: 94 - protein, 560 aa, Influenza virus
MSNMDIDGINTGTIDKTPEEITSGTSGTTRPIIRPATLAPPSNKRTRNPSPERATTSSEADVGRKTQKKQTPTEIKKSVYNMVVKLGEFYNQMMVKAGLNDDMERNLIQNAHAVERILLAATDDKKTEFQRKKNARDVKEGKEEIDHNKTGGTFYKMVRDDKTIYFSPIRITFLKEEVKTMYKTTMGSDGFSGLNHIMIGHSQMNDVCFQRSKALKRVGLDPSLISTFAGSTLPRRSGATGVAIKGGGTLVAEAIRFIGRAMADRGLLRDIKAKTAYEKILLNLKNKCSAPQQKALVDQVIGSRNPGIADIEDLTLLARSMVVVRPSVASKVVLPISIYAKIPQLGFNVEEYSMVGYEAMALYNMATPVSILRMGDDAKDKSQLFFMSCFGAAYEDLRVLSALTGIEFKPRSALKCKGFHVPAKEQVEGMGAALMSIKLQFWAPMTRSGGNEVGGDGGSGQISCSPVFAVERPIALSKQAVRRMLSMNIEGRDADVKGNLLKMMNDSMAKKTNGNAFIGKKMFQISDKNKTNPVEIPIKQTIPNFFFGRDTAEDYDDLDY

SEQ ID NO: 95 - protein, 248 aa, Influenza virus; identical to SEQ ID NO: 87

SEQ ID NO: 96 - protein, 109 aa, Influenza virus
MLEPFQILSICSFILSALHFMAWTIGHLNQIKRGVNMKIRIKNPNKETINREVSILRHSYQKEIQAKETMKEVLSDNMEVLSDHIVIEGLSAEEIIKMGETVLEVEELH

SEQ ID NO: 97 - protein, 281 aa, Influenza virus
MADNMTTTQIEVGPGATNATINFEAGILECYERLSWQRALDYPGQDRLNKLKRKLESRIKTHNKSEPESKRMSLEERKAIGVKMMKVLLFMNPSAGVEGFEPYCMKNPSNSNCPDCNWADYPPTPGKYLDGIEEEPENVGDSTEIVLRDMNNKDARQKIKEEVNTQKEGKFRLTIKRDIRNVLSLRVLVNGTFIKHPNGYKSLSTLHRLNAYDQSGRLVAKLVATDDLTVEDEEDGHRILNSLFERLNEGHSKPIRAAETAVGVLSQFGQEHRLSPEERDN

SEQ ID NO: 98 - protein, 122 aa, Influenza virus
MADNMTTTQIEWRMKKMAIGSSTHSSSVLMKDIQSQFEQLKLRWESYPNLVKSTDYHQKRETIRLVTEELYLLSKRIDDNILFHKTVIANSSIIADMIVSLSLLETLYEMKDVVEVYSRQCL

SEQ ID NO: 99 - protein, 469 aa, Influenza virus; identical to SEQ ID NO: 52

SEQ ID NO: 100 - DNA, 1812 bp, Influenza virus
agcattttcttgtgagcttcgagcactaataaaactgaaaatcaaaatgtccaacatggatattgacagtataaataccggaacaatcgataaaaaaccagaagaactgactcccggaaccagtggggcaaccagaccaatcatcaagccagcaacccttgctccgccaagcaacaaacgaacccgaaatccatccccagaaaggacaaccacaagcagtgaaaccgatatcggaaggaaaatccaaaagaaacaaaccccaacagagataaagaagagcgtctacaacatggtggtaaagctgggtgaattctacaaccagatgatggtcaaagctggacttaatgatgacatggaaaggaatctaatccaaaatgcacaagctgtggagagaatcctattggctgcaactgatgacaagaaaactgaataccaaaagaaaaggaatgccagagatgtcaaagaagggaaggaagaaatagaccacaacaagacaggaggcaccttttataagatggtaagagatgataaaaccatctacttcagccctataaaaattacctttttaaaagaagaggtgaaaacaatgtacaagaccaccatggggagtgatggtttcagtggactaaatcacattatgattggacattcacagatgaacgatgtctgtttccaaagatcaaaggcactgaaaagggttggacttgacccttcattaatcagtacttttgccggaagcacactacccagaagatcaggtacaactggtgttgcaatcaaaggaggtggaactttagtggcagaagccattcgatttataggaagagcaatggcagacagagggctactgagagacatcaaggccaagacagcctatgaaaagattcttctgaatctgaaaaacaagtgctctgcgccccaacaaaaggctctagttgatcaagtgatcggaagtaggaacccagggattgcagacatagaagacctaactctgcttgccagaagcatgatagttgtcagaccctctgtagcgagcaaagtggtgcttcccataagcatttatgctaaaatacctcaactaggattcaatatcgaagaatactctatggttgggtatgaagccatggctctttataatatggcaacacctgtttccatattaagaatgggagatgacgcaaaagataaatctcaactattcttcatgtcgtgcttcggagctgcctatgaagatctaagagtgttatctgcactaacgggcaccgaatttaagcctagatcagcactaaaatgcaagggtttccatgtcccggctaaggagcaagtagaaggaatgggggcagctctgatgtccatcaagcttcagttctgggccccaatgaccagatctggagggaatgaagtaagtggagaaggagggtctggtcaaataagttgcagccctgtgtttgcagtagaaagacctattgctctaagcaagcaagctgtaagaagaatgctgtcaatgaacgttgaaggacgtgatgcagatgtcaaaggaaatctactcaaaatgatgaatgattcgatggcaaagaaaaccagtggaaatgctttcattgggaagaaaatgtttcaaatatcagacaaaaacaaagtcaatcccattgagattccaattaagcagaccatccccagtttcttctttgggagggacacagcagaggattatgatgacctcgattattaaagcaataaaatagacactatggctgtgactgtttcagtacgtttgggatgtgggtgtttactcttattgaaataaatgtaaaa

SEQ ID NO: 101 - protein, 560 aa, Influenza virus
MSNMDIDGINTGTIDKTPEEITSGTSGATRPIIKPATLAPPSNKRTRNPSPERATTSSEAIVGRRTQKKQTPTEIKKSVYNMVVKLGEFYNQMMVKAGLNDDMERNLIQNAHAVERILLAATDDKKTEYQKKKNARDVKEGKEEIDHNKTGGTFYKMVRDDKTIYFSPIRITFLKEEVKTMYKTTMGSDGFSGLNHIMIGHSQMNDVCFQRSKALKRVGLDPSLISTFAGSTLPRRSGATGVAIKGGGTLVAEAIRFIGRAMADRGLLRDIRAKTAYEKILLNLKNKCSAPQQKALVDQVIGSRNPGIADIEDLTLLARSMVVVRPSVASKVVLPISINAKIPQLGFNVEEYSMVGYEAMALYNMATPVSILRMGDDAKDKSQLFFMSCFGAAYEDQRVLSALTGTEFKPRSALKCKGFHVPAKEQVEGMGAALMSIKLQFWAPMTRSGGNEVGGDGGSGQISCSPVFAVERPIALSKQAVRRMLSMNIEGRDADVKGNLLKMMNDSMAKKTNGNAFIGKKMFQISDKNKINPVDIPIKQTIPNFFFGRDTAEDYDDLDY

SEQ ID NO: 102 - protein, 560 aa, Influenza virus
MSNMDIDGINTGTIDKTPEEITSGTSGATRPIIKPATLAPPSNKRTRNPSPERAATSSEADVGRRTQKKQTPTEIKKSVYNMVVKLGEFYNQMMVKAGLNDDMERNLIQNAHAAERILLAATDDKKTEFQKKKNARDVKEGKEEIDHNKTGGTFYKMVRDDKTIYFSPIRITFLKEEVKTMYKTTMGSDGFSGLNHIMIGHSQMNDVCFQRSKALKRVGLDPSLISTFAGSTLPRRSGATGVAIKGGGTLVAEAIRFIGRAMADRGLLRDIRAKTAYEKILLNLKNKCSAPQQKALVDQVIGSRNPGIADIEDLTLLARSMVVVRPSVASKVVLPISINAKIPQLGFNVEEYSMVGYEAMALYNMATPVSILRMGDDAKDKSQLFFMSCFGAAYEDQRVLSALTGTEFKHRSALKCKGFHVPAKEQVEGMGAALMSIKLQFWAPMTRSGGNEVGGDGGSGQISCSPVFAVERPIALSKQAVRRMLSMNIEGRDADVKGNLLKMMNDSMTKKTNGNAFIGKKMFQISDKNKTNPIEIPIKQTIPNFFFGRDTAEDYDDLDY

SEQ ID NO: 103 - DNA, 1842 bp, Influenza virus
agcagaagcacagcattttcttgtgaacttcaagtaccaacaaaaactgaaaatcaaaatgtccaacatggatattgacggcatcaacactggaacaattgacaaaacaccagaagaaataacttccggaaccagtggggcaaccagaccaatcatcaagccagcaacccttgccccaccaagcaataaacgaacccgaaacccatccccagaaagggcaaccacaagcagcgaagcgattgtcggaaggagaacccaaaagaaacaaaccccgacagagataaagaagagcgtctacaatatggtagtgaaactgggtgaattctacaaccagatgatggtcaaagctggactcaacgatgacatggagagaaacctaatccaaaatgcacatgctgtggaaagaattctattggctgctactgatgacaagaaaactgaataccaaaagaaaaagaatgccagagatgtcaaagaagggaaagaagaaatagaccacaacaaaacaggaggcaccttttataagatggtaagagatgataaaaccatctacttcagccctataagaattacctttttaaaagaagaggtgaaaacaatgtacaagaccaccatggggagtgatggtttcagtggactaaatcacatcatgattgggcattcacagatgaacgatgtctgtttccaaagatcaaaggcactaaaaagagttggacttgacccttcattaatcagtacttttgcaggaagcacactccccagaagatcaggtgcaactggtgttgcgatcaaaggaggtggaactttagtggcagaagccattcgatttataggaagagcaatggcagacagagggctattgagagacatcagagccaagacggcctatgaaaagattcttctgaatctgaaaaacaagtgctctgcgccccaacaaaaggctctagttgatcaagtgatcggaagtagaaacccagggattgcagacatagaagacctaaccctgcttgcccgaagcatggtcgttgtcaggccctctgtagcgagcaaagtggtgcttcccataagcattaatgctaaaatacctcaactagggttcaatgttgaagaatactctatggttgggtatgaagccatggctctttataatatggcaacacctgtttccatattaagaatgggagacgatgcaaaagataaatcacaattattcttcatgtcttgctttggagctgcctatgaagaccaaagagttttgtctgcactaaccggcacagaattcaagcctaggtcagcattaaagtgcaagggtttccacgttccagcaaaggagcaagtggaaggaatgggggcagctctgatgtccatcaagctccagttttgggccccaatgaccagatctggggggaacgaagtaggtggagacggagggtctggtcaaataagttgcagccccgtgtttgcagtagagagacctattgctctaagcaagcaagctgtaagaagaatgctgtcaatgaatattgagggacgtgatgcagatgtcaaaggaaatctactcaagatgatgaatgattcaatggctaagaaaaccaatggaaatgctttcattgggaagaaaatgtttcaaatatcagacaaaaacaaaatcaatcccgttgatattccaattaagcagaccatccccaatttcttctttgggagggacacagcagaggattatgatgacctcgattattaaagcaacaaaatagacactatggctgtgactgtttcagtacgtttggaatgtgggtgtttactcttattgaaataaatgtaaaaaatgctgttgtttctact

SEQ ID NO: 104 - DNA, 1842 bp, Influenza virus
agcagaagcacagcattttcttgtgaacttcaagtaccaacaaaaactgaaaatcaaaatgtccaacatggatattgacggcatcaacactggaacaattgacaaaacaccagaagaaataacttccggaaccagtggggcaaccagaccaatcatcaaaccagcaacccttgccccaccaagcaacaaacgaacccgaaacccatccccggaaagggcagccacaagcagtgaagctgatgtcggaaggagaacccaaaagaaacaaaccccgacagagataaagaagagcgtctacaatatggtagtgaaactgggtgaattctacaaccagatgatggtcaaagctggactcaacgatgacatggagagaaacctaatccaaaatgcacatgctgcggaaagaattctattggctgctactgatgacaagaaaactgaattccaaaagaaaaagaatgccagagatgtcaaagaagggaaagaagaaatagaccacaacaaaacaggaggcaccttttacaagatggtaagagatgataaaaccatctacttcagccctataagaattacctttttaaaagaagaggtgaaaacaatgtacaaaaccaccatggggagtgatggtttcagtggactaaatcacatcatgattgggcattcacagatgaacgatgtctgtttccaaagatcaaaggcactaaaaagagttggacttgacccttcattaatcagtacttttgcaggaagcacactccccagaagatcaggtgcaactggtgttgcgatcaaaggaggtggaactttagtggcagaagccattcgatttataggaagagcaatggcagacagagggctattgagagacatcagagccaagacggcctatgaaaagattcttctgaatctgaaaaacaagtgctctgcgccccaacaaaaggctctagttgatcaagtgatcggaagtagaaatccagggattgcagacatagaagacctaaccctgcttgcccgaagcatggtcgttgtcaggccctctgtagcgagcaaagtggtgcttcccataagcattaatgccaaaatacctcaactagggttcaatgttgaagaatactctatggttgggtatgaagccatggctctttataatatggcaacacctgtttccatattaagaatgggagacgatgcaaaagataaatcacaattattcttcatgtcttgcttcggagctgcctatgaagaccaaagagttttgtctgcactaacaggcacagaattcaagcataggtcagcattaaagtgcaagggtttccacgttccagcaaaggagcaagtggaaggaatgggggcagctctgatgtccatcaagctccagttttgggctccaatgaccagatctggggggaatgaagtaggtggagacggagggtctggtcaaataagttgcagccccgtgtttgcagtagaaagacctattgctctaagcaagcaagctgtaagaagaatgctgtcaatgaatattgagggacgtgatgcagatgtcaaaggaaatctactcaagatgatgaatgattcaatgactaagaaaaccaatggaaatgctttcattgggaagaaaatgtttcaaatatcagacaaaaacaaaaccaatcccattgagattccaattaagcagaccatccccaatttcttctttgggagggacacagcagaggattatgatgacctcgattattaaagcaacaaaatagacactatggctgtgactgtttcagtacgtttggaatgtgggtgtttacttttattgaaataaatgtaaaaaatgctgttgtttctact

(19) United States
(12) Patent Application Publication
(10) Pub. No.: US 2020/0172877 A1
(43) Pub. Date: Jun. 4, 2020

(54) INFLUENZA VIRUS REASSORTMENT

(71) Applicants: Seqirus UK Limited, Berkshire (GB); Synthetic Genomics, Inc., La Jolla, CA (US)

(72) Inventors: Philip DORMITZER, Cambridge, MA (US); Peter MASON, Cambridge (CA); Pirada SUPHAPHIPHAT, Cambridge, MA (US); Daniel GIBSON, La Jolla, CA (US); David WENTWORTH, La Jolla, CA (US); Timothy STOCKWELL, La Jolla, CA (US); John GLASS, La Jolla, CA (US)

(73) Assignees: Seqirus UK Limited, Berkshire (GB); Synthetic Genomics, Inc., La Jolla, CA (US)

(21) Appl. No.: 16/583,390

(22) Filed: Sep. 26, 2019

Related U.S. Application Data
(63) Continuation of application No. 16/276,992, filed on Feb. 15, 2019, now abandoned.
Application No. 16/276,992 is a continuation of application No. 16/011,994, filed on Jun. 19, 2018, now abandoned.
Application No. 16/011,994 is a continuation of application No. 14/762,439, filed on Jul. 21, 2015, now Pat. No. 10,030,231.
Application No. 14/762,439 is a continuation of application No. 13/841,752, filed on Mar. 15, 2013, now abandoned.
(60) Provisional application No. 61/849,325, filed on Jan. 23, 2013.

(30) Foreign Application Priority Data
Mar. 15, 2013 (GB) 1304827.7

(51) Int. Cl. C12N 7/00; C12N 5/071; A61K 39/145; C07K 14/005

(52) U.S. Cl. CPC C12N 7/00; C12N 5/0686; A61K 39/145; A61K 2039/525; C12N 2510/02; C12N 2760/16121; C12N 2760/16221; C07K 14/005

(57) ABSTRACT

Improved methods for the production of reassortant influenza viruses are provided.

This application claims the benefit of U.S. provisional application 61/849,325 (filed 23 Jan. 2013), U.S. Ser. No. 13/841,752 (filed 15 Mar. 2013) and GB 1304827.7 (filed 15 Mar. 2013), the complete contents of which are hereby incorporated herein by reference for all purposes.

### STATEMENT OF GOVERNMENT SUPPORT

This invention was supported in part with Government support under BARDA Contract No. HHSO100201000061C awarded by Office of Public Health Emergency Preparedness, Biomedical Advanced Research and Development Authority. The Government has certain rights in the invention.

The influenza virus sequence database used for UTR construction and the generation of a library of synthetic gene segments was funded in part by the National Institute of Allergy and Infectious Diseases, National Institutes of Health, Department of Health and Human Services under Contract No. HHSN272200900007C.

### TECHNICAL FIELD

This invention is in the field of influenza virus reassortment. Furthermore, it relates to manufacturing vaccines for protecting against influenza viruses.

### BACKGROUND ART

The 2009 H1N1 influenza pandemic response was the fastest global vaccine development effort in history. Within six months of the pandemic declaration, vaccine companies had developed, produced, and distributed hundreds of millions of doses of licensed pandemic vaccines. Unfortunately, the response was not fast enough as substantial vaccine quantities were available only after the second pandemic wave had peaked. This delay was at least partially due to the late availability of a high-yielding influenza strain which could be used for vaccine production.

One way of obtaining a high-yielding influenza strain is to reassort the circulating vaccine strain with a faster-growing high-yield donor strain. This can be achieved by co-infecting a culture host with the circulating influenza strain and the high-yield donor strain and selecting for reassortant viruses which contain the hemagglutinin (HA) and neuraminidase (NA) segments from the vaccine strain and the other viral segments (i.e. those encoding PB1, PB2, PA, NP, M₁, M₂, NS₁ and NS₂) from the donor strain. Another approach is to reassort the influenza viruses by reverse genetics (see, for example references 1 and 2).

As the 2009 experience has shown, the traditional methods for reassorting influenza viruses may not be fast enough to provide sufficient amounts of influenza vaccine during a pandemic. In particular, valuable time is lost in preparing the high-yielding seed virus. There is therefore still a need in the art to provide methods which allow the rapid generation of high-yielding seed viruses in order to further decrease the time it takes between the emergence of an influenza pandemic and the provision of an influenza vaccine. The prior art had suggested solving this problem by preparing HA segments synthetically (see, for example, references 3, 4 and 5). The fastest reported time frame in which the influenza viruses can be prepared using these methods is nine days. Furthermore, these techniques rely on the use of 293T cells which have a high transfection efficacy but which are not approved for vaccine manufacture. There is therefore a need in the art to provide further and improved methods for preparing reassortant influenza viruses.

### SUMMARY OF PREFERRED EMBODIMENTS

In some aspects, the invention provides methods which allow a faster preparation of influenza viruses. For example, the invention provides a method of preparing an influenza virus, comprising the steps of (a) preparing one or more expression construct(s) which comprise(s) coding sequences for expressing at least one segment of an influenza virus genome; (b) introducing into a cell which is not 293T one or more expression construct(s) which encode(s) the viral segments of an influenza virus, wherein at least one expression construct is the expression construct prepared in step (a); and (c) culturing the cell in order to produce a reassortant influenza virus from the expression construct(s) introduced in step (b); wherein steps (a) to (c) are performed in a time period of 124 hours or less. The cell is preferably a non-human cell or a human non-kidney cell.

Also provided is a method of preparing an influenza virus comprising the steps of (a) preparing one or more expression construct(s) which comprise(s) coding sequences for expressing at least one segment of an influenza virus genome; (b) introducing into a cell one or more expression construct(s) which encode(s) the viral segments of an influenza virus, wherein at least one expression construct is the expression construct prepared in step (a); and (c) culturing the cell in order to produce a reassortant influenza virus from the expression construct(s) introduced in step (b); wherein steps (a) to (c) are performed in a time period of 100 hours or less.

The invention also provides a method of preparing an influenza virus comprising the steps of (a) providing a synthetic expression construct which comprises coding sequences for expressing at least one segment of an influenza virus genome by (i) synthesising a plurality of overlapping fragments of the synthetic expression construct, wherein the overlapping fragments span the complete synthetic expression construct, and (ii) joining the fragments to provide the synthetic expression construct; (b) introducing into a cell which is not 293T one or more expression construct(s) which encode(s) the viral segments of an influenza virus, wherein at least one expression construct is the synthetic expression construct prepared in step (a); and (c) culturing the cell in order to produce a reassortant influenza virus from the viral segments introduced in step (b); wherein steps (a) to (c) are performed in a time period of 124 hours or less. The cell is preferably a non-human cell or a human non-kidney cell.

The methods may further comprise a step (d) contacting a cell which is of the same cell type as the cell used in step (c) with the virus produced in step (b) to produce further reassortant influenza virus.

The invention also provides a method of preparing an influenza virus, comprising the steps of (a) providing a synthetic expression construct which comprises coding sequences for expressing at least one segment of an influenza virus genome by (i) synthesising a plurality of overlapping fragments of the synthetic expression construct, wherein the overlapping fragments span the complete synthetic expression construct, and (ii) joining the fragments to provide the synthetic expression construct; (b) introducing into a cell one or more expression construct(s) which encode(s) the viral segments of an influenza virus, wherein at least one expression construct is the synthetic expression construct prepared in step (a); (c) culturing the cell in order to produce a reassortant influenza virus from the viral segments introduced in step (b); and (d) contacting a cell which is of the same cell type as the cell used in step (c) with the virus produced in step (c) to produce further reassortant influenza virus; wherein steps (a) to (c) are performed in a time period of 124 hours or less. The cell used is preferably not 293T.

Further provided is a method of preparing an influenza vaccine, comprising the steps of (a) contacting a cell with the reassortant influenza virus prepared by a method according to the invention; (b) culturing the cell in order to produce an influenza virus; and (c) preparing a vaccine from the influenza virus produced in step (b). The cell used in the method is preferably a human non-kidney cell or a non-human cell. Alternatively, or in addition, the cell used in step (a) is of the same cell type as the cell which was used to rescue the influenza virus in the methods discussed in the preceding paragraphs. This is preferred because it facilitates regulatory approval, avoids conflicting culture conditions and avoids the need to retain two different cell types. The cell used is preferably not 293T as this cell is not approved for human vaccine manufacture.

The invention also provides a method of preparing a synthetic expression construct which encodes a viral segment from an influenza virus, comprising: (a) providing the sequence of at least part of the coding region of the HA or NA segment from an influenza virus; (b) identifying the HA and/or NA subtype of the influenza virus from which the coding region is derived; (c) providing a UTR sequence from an influenza virus with the same HA or NA subtype as the subtype identified in step (b); and (d) preparing a synthetic expression construct which encodes a viral segment comprising the coding sequence and the UTR.

The Synthetic Expression Construct

The synthetic expression construct is a DNA molecule which comprises coding sequences for expressing one or more viral RNA segment(s) of an influenza virus genome. The encoded segments can be expressed and then function as viral RNAs which can be packaged into virions to give recombinantly expressed virus. Thus the synthetic expression construct is suitable for producing an influenza virus by reverse genetics, either alone or in combination with other expression constructs.

The synthetic expression construct can be produced by (i) synthesising a plurality of overlapping fragments of the synthetic expression construct, wherein the overlapping fragments span the complete synthetic expression construct, and (ii) joining the fragments to provide the synthetic expression construct.

The method can involve notionally splitting the desired DNA sequence into fragments which can be prepared by a chosen DNA synthesis method e.g. by phosphoramidite chemistry. References 6 and 7 report that the entire 16,299 base pair mouse mitochondrial genome could be synthesized from 600 overlapping 60-base oligonucleotides. The method uses Phusion DNA polymerase (New England Biolabs [NEB]), T5 exonuclease (Epicentre) and Taq DNA ligase (NEB) to join multiple DNA fragments during a brief 50° C. reaction (6). The inventors have discovered that this method can be used to generate synthetic DNA copies of the influenza virus genome and that the resulting method is particularly advantageous because it is rapid and readily automated. Joining the fragments in step (ii) of the methods described above can thus comprise contacting the fragments with a DNA polymerase and a DNA ligase. The method can be practised with any DNA polymerase which can amplify DNA, including Phusion™ DNA polymerase and Taq DNA™ polymerase. Preferably, the methods use a high fidelity DNA polymerase, such as Phusion™ DNA polymerase, PFU™, AccuPrime™ Taq DNA Polymerase, AMPLITAQ™ GOLD DNA pol, T5 DNA polymerase, phi29 DNA polymerase, VENTR™ DNA pol, Deep Vent DNA pol. etc. This is preferred because it decreases the error rate of the resulting DNA molecule. Suitable DNA ligases are also known to the skilled person and include Taq™ DNA ligase, AMPLIGASE thermostable DNA ligase, and Tfi ligase. Reference 8 also discusses suitable ligases which can be used.

Suitable buffers and reaction conditions are described in references 6 and 7 and are also known to the skilled person. The methods can be performed at a temperature between 40° C. and 60° C., for example at a temperature between 45° C. and 55° C. or at a temperature of about 50° C. Preferably, the fragments are incubated with the DNA polymerase and the DNA ligase for a time period of between 15 and 60 minutes.

The synthetic expression constructs may be assembled from fragments with a size of about 30 nucleotides, at least 30 nucleotides, 40-60 nucleotides or at least 61 nucleotides. The fragments may also have a length of less than 40 nucleotides, less than 50 nucleotides, less than 60 nucleotides, less than 100 nucleotides, less than 200 nucleotides, less than 500 nucleotides, less than 1000 nucleotides, less than 5000 nucleotides, or less than 10000 nucleotides. Preferably, the synthetic expression constructs are assembled from fragments with a size of between 61 and 100 nucleotides, for example between 61 and 74 nucleotides. Such fragments are longer than the fragments used in the prior art.

For example, references 6 and 7 used fragments with a length of 60 nucleotides. By using longer fragments, the inventors found that the speed for obtaining synthetic expression constructs was increased. This was unexpected as a skilled person would have expected longer fragments to be thermodynamically unfavourable and that it would be harder for overlaps to anneal to each other.

The fragments are synthesised and joined to give the synthetic expression constructs. This can be achieved by performing more than one joining (e.g. ligation) step. For example, some of the DNA fragments may be joined to give longer fragments, and these longer fragments can then be joined again, etc. until the complete synthetic expression construct is eventually prepared. Where the molecule is assembled step-wise in this fashion, the fragments at each stage may be maintained as inserts in vectors e.g. in plasmids or BAC or YAC vectors.

The synthetic expression construct may also be assembled using a single joining step (e.g. a single ligation step) and this is preferred because it allows for a faster assembly of the synthetic expression construct. In these embodiments, fragments which span the entire synthetic expression construct are treated with a joining agent (e.g. a DNA ligase) which assembles the whole synthetic expression construct in a single reaction.

The fragments can be designed to overlap, thereby facilitating the assembly in the correct order and this is preferred when the synthetic expression construct is assembled in a single joining step. It is preferred that the fragments overlap by at least 15 nucleotides, at least 20 nucleotides, at least 40 nucleotides or at least 60 nucleotides. This is preferred because the inventors have found that this increased overlap allowed rapid synthesis of the fragments with high accuracy. Thus the method may involve the synthesis of a plurality of overlapping fragments of the desired synthetic expression construct, such that the overlapping fragments span the complete synthetic expression construct. Both ends of each fragment overlap with a neighbouring 5′ or 3′ fragment, except for the terminal fragments of a linear molecule where no overlap is required (but if a circular molecule is desired, the two terminal fragments may overlap). Assembly of fragments during the synthetic process can involve in vitro and/or in vivo recombination. For in vitro methods, digestion with a 3′ exonuclease can be used to expose overhangs at the terminus of a fragment, and complementary overhangs in overlapping fragments can then be annealed, followed by joint repair (“chewback assembly”). For in vivo methods, overlapping clones can be assembled using e.g. the TAR cloning method disclosed in reference 9. For fragments <100 kbp (e.g. easily enough to encode all segments of an influenza virus genome) it is readily possible to rely solely on in vitro recombination methods.

Other synthetic methods may be used. For instance, reference 10 discloses a method in which fragments of about 5 kbp are synthesised and then assembled into longer sequences by conventional cloning methods. Unpurified 40 base synthetic oligonucleotides are built into 500-800 bp synthons by automated PCR-based gene synthesis, and these synthons joined into multisynthon ˜5 kbp segments using a small number of endonucleases and “ligation by selection.” These large segments can subsequently be assembled into longer sequences by conventional cloning. This method can readily provide a 32 kbp DNA molecule, which is easily enough to encode a complete influenza virus. Similarly, reference 11 discloses a method where a 32 kb molecule was assembled from seven DNA fragments which spanned the complete sequence. The ends of the seven DNAs were engineered with unique junctions, thereby permitting assembly only of adjacent fragments. The interconnecting restriction site junctions at the ends of each DNA are systematically removed after assembly.

Following the assembly of the synthetic expression construct, it is possible to amplify the whole or part of the synthetic expression construct. Methods for DNA amplification are known in the art and include, for example, polymerase chain reaction (PCR). Where only part of the synthetic expression construct is amplified it is preferred to amplify the part of the expression construct which encodes the one or more viral segments.

One drawback of the reference 6 method is that only 3% of the synthetic products have the correct sequence. In the prior art this problem was solved by cloning and sequencing subassemblies, and sets of error-free sequences were selected for subsequent rounds of assembly. Whilst this addresses the problem of errors in the resulting DNA molecule, the method is time-consuming and thus not suitable for use in a method which requires high speed and accuracy. The inventors have thus addressed the problem of error correction differently. In particular, they have discovered that the error rate can be decreased significantly by including an alternative error correction step. The invention thus provides a method of preparing a synthetic expression construct, comprising the steps of (i) synthesising a plurality of overlapping fragments of the synthetic expression construct, wherein the overlapping fragments span the complete synthetic expression construct, (ii) joining the fragments to provide a DNA molecule; (iii) melting the DNA molecule; (iv) re-annealing the DNA in the presence of an agent which excises mismatched nucleotides from the DNA molecule; and (v) amplifying the DNA to produce the synthetic expression construct. By including this additional step, the inventors were able to obtain full-length sequences in which 80-100% had the correct sequence. The DNA in step (v) can be amplified using DNA polymerases, preferably high-fidelity DNA polymerases, as known in the art and described above.

Suitable conditions for melting (i.e. dissociating the DNA double helix into single strands) and re-annealing DNA are known in the art. For example, the DNA can be melted by heating it to a temperature of at least 90° C. Likewise, the DNA can be re-annealed by reducing the temperature. The agent used to excise mismatched nucleotides is usually an enzyme such as, for example, the Res 1 enzyme (which is available in the ErrASE™ error correction kit (Novici Biotech)), Cel I, T7 endonuclease I, S1 nuclease, T7 endonuclease, E. coli endo. V, Mung Bean endo., etc.

A synthetic expression construct may include one or more “watermark” sequences. These are sequences which can be used to identify or encode information in the DNA. It can be in either noncoding or coding sequences. Most commonly, it encodes information within coding sequences without altering the amino acid sequences. For DNAs encoding segmented RNA viral genomes, any watermark sequences are ideally included in intergenic sites because synonymous codon changes may have substantial biological effects for encoded RNA segments.

The synthetic expression construct may be linear (14) or circular. Circular synthetic expression constructs can be made by circularising linear constructs and vice versa. Methods for such circularisation are described in ref. 14. Linearisation of a circular molecule can be achieved in various easy ways e.g. by utilising one or more restriction enzyme(s), or by amplification from a template (including a circular template) using a nucleic acid amplification technique (e.g. by PCR).

Where the synthetic expression construct is circular, it is possible to contact the DNA following step (ii) with an agent (for example an enzyme) that degrades linear DNA. This has the advantage that linear synthetic expression constructs are selectively removed, thus selecting for the circular product. Suitable agents are known in the art and include, for example, T5 exonuclease, lambda exonuclease, and exonuclease III.

The synthetic expression construct may be incorporated into a vector, such as a plasmid or other episomal construct, using conventional techniques known in the art. The 3′ and/or 5′ terminal fragment of the synthetic expression construct may comprise an overhang which is complementary to an overhang on the vector, which facilitates the cloning of the synthetic expression construct (such that, for example, the synthetic expression construct may be cloned into an overhang created by a restriction enzyme). The vector may provide the regulatory sequences which are necessary to express the viral RNA segments from the DNA construct (e.g. RNA pol I promoter, RNA pol II promoter; RNA polymerase I transcription termination sequence, RNA polymerase II transcription termination sequence etc.). This can be advantageous because these sequences do then not need to be included in the synthetic expression construct. It is also possible to clone a synthetic expression construct without regulatory sequences into a vector that provides these sequences and subsequently amplifying a linear synthetic expression construct which comprises the original synthetic expression construct in conjunction with the regulatory sequences so that the resulting synthetic expression construct can then be used to express the viral segments.

Expression Constructs

The invention produces influenza viruses through reverse genetics techniques. In these techniques, the viruses may be produced in culture hosts using a synthetic expression construct which comprises coding sequences for expressing at least one segment of an influenza virus genome, as described in the preceding sections. The synthetic expression construct can drive expression in a eukaryotic cell of viral segments encoded therein. The expressed viral segment RNA can be translated into a viral protein that can be incorporated into a virion.

The term “synthetic expression construct” refers to an expression construct which has been prepared synthetically as described in the preceding sections, or which is derived from an expression construct prepared in this manner (for example by DNA amplification). It also encompasses vectors which comprise such an expression construct. The term “expression construct” encompasses both synthetic expression construct as well as expression constructs which were not prepared synthetically.

The synthetic expression construct may encode all the viral segments which are necessary to produce an influenza virus. Alternatively, it may encode one, two, three, four, five, six, or seven viral segments. Where the synthetic expression construct does not encode all the viral segments which are necessary to produce an influenza virus, the remaining viral segments are provided by one or more further expression construct(s). These one or more further expression constructs may also be synthetic expression constructs or they may be expression constructs which have been generated using alternative methods such as, for example, the methods described in reference 12.

Where the synthetic expression construct does not encode all the viral segments which are necessary to produce an influenza virus, the synthetic expression construct may encode the neuraminidase (NA) and/or hemagglutinin (HA) segments and the remaining vRNA encoding segments, excluding the HA and/or NA segment(s), are included on a different expression construct. This has the advantage that only the expression construct comprising the HA and/or NA segments needs to be replaced when a new influenza vaccine strain emerges (e.g. a new pandemic influenza virus or a new seasonal influenza virus).

The expression constructs may be uni-directional or bi-directional expression constructs. Where a host cell expresses more than one transgene (whether on the same or different expression constructs) it is possible to use uni-directional and/or bi-directional expression.

Bi-directional expression constructs contain at least two promoters which drive expression in different directions (i.e. both 5′ to 3′ and 3′ to 5) from the same construct. The two promoters can be operably linked to different strands of the same double stranded DNA. Preferably, one of the promoters is a pol I promoter and at least one of the other promoters is a pol II promoter. This is useful as the pol I promoter can be used to express uncapped vRNAs while the pol II promoter can be used to transcribe mRNAs which can subsequently be translated into proteins, thus allowing simultaneous expression of RNA and protein from the same construct.

The pol I and pol II promoters used in the expression constructs may be endogenous to an organism from the same taxonomic order from which the host cell is derived. Alternatively, the promoters can be derived from an organism in a different taxonomic order than the host cell. The term “order” refers to conventional taxonomic ranking, and examples of orders are primates, rodentia, carnivora, marsupialia, cetacean, etc. Humans and chimpanzees are in the same taxonomic order (primates), but humans and dogs are in different orders (primates vs. carnivora). For example, the human pol I promoter can be used to express viral segments in canine cells (e.g. MDCK cells) [13].Where more than one expression construct is used within an expression system, the promoters may be a mixture of endogenous and non-endogenous promoters.

The expression construct will typically include an RNA transcription termination sequence. The termination sequence may be an endogenous termination sequence or a termination sequence which is not endogenous to the host cell. Suitable termination sequences will be evident to those of skill in the art and include, but are not limited to, RNA polymerase I transcription termination sequence, RNA polymerase II transcription termination sequence, and ribozymes. Furthermore, the expression constructs may contain one or more polyadenylation signals for mRNAs, particularly at the end of a gene whose expression is controlled by a pol II promoter.

An expression construct may be a vector, such as a plasmid or other episomal construct. Such vectors will typically comprise at least one bacterial and/or eukaryotic origin of replication. Furthermore, the vector may comprise a selectable marker which allows for selection in prokaryotic and/or eukaryotic cells. Examples of such selectable markers are genes conferring resistance to antibiotics, such as ampicillin or kanamycin. The vector may further comprise one or more multiple cloning sites to facilitate cloning of a DNA sequence.

As an alternative, an expression construct may be a linear expression construct. Such linear expression constructs will typically not contain any amplification and/or selection sequences. However, linear constructs comprising such amplification and/or selection sequences are also within the scope of the present invention. An example of a method using such linear expression constructs for the expression of influenza virus is described in reference 14.

Where the expression construct is a linear expression construct, it is possible to linearise it before introduction into the host cell utilising a single restriction enzyme site. Alternatively, it is possible to excise the expression construct from a vector using at least two restriction enzyme sites. Furthermore, it is also possible to obtain a linear expression construct by amplifying it using a nucleic acid amplification technique (e.g. by PCR).

Where the expression construct is not a synthetic expression construct, it may be generated using methods known in the art. Such methods were described, for example, in reference 15.

The expression constructs of the invention can be introduced into host cells using any technique known to those of skill in the art. For example, expression constructs of the invention can be introduced into host cells by employing electroporation, DEAE-dextran, calcium phosphate precipitation, liposomes, microinjection, or microparticle-bombardment. Once transfected, the host cell will recognise genetic elements in the construct and will begin to express the encoded viral RNA segments.

The expression construct(s) can be introduced into the same cell type which is subsequently used for the propagation of the influenza viruses. Alternatively, the cells into which the expression constructs are introduced and the cells used for propagation of the influenza viruses may be different. In some embodiments, cells may be added following the introduction of the expression construct(s) into the cell, as described in reference 16. This is particularly preferred because it increases the rescue efficiency of the viruses further and can thus help to reduce the time required for viral rescue. The cells which are added may be of the same or a different cell type as the cell into which the expression construct(a) is/are introduced, but it is preferred to use cells of the same cell type as this facilitates regulatory approval and avoids conflicting culture conditions.

Where the expression host is a canine cell, such as a MDCK cell line, protein-coding regions may be optimised for canine expression e.g. using a promoter from a wild-type canine gene or from a canine virus, and/or having codon usage more suitable for canine cells than for human cells. For instance, whereas human genes slightly favour UUC as the codon for Phe (54%), in canine cells the preference is stronger (59%). Similarly, whereas there is no majority preference for Ile codons in human cells, 53% of canine codons use AUC for Ile. Canine viruses, such as canine parvovirus (a ssDNA virus) can also provide guidance for codon optimisation e.g. 95% of Phe codons in canine parvovirus sequences are UUU (vs. 41% in the canine genome), 68% of Ile codons are AUU (vs. 32%), 46% of Val codons are GUU (vs. 14%), 72% of Pro codons are CCA (vs. 25%), 87% of Tyr codons are UAU (vs. 40%), 87% of His codons are CAU (vs. 39%), 92% of Gln codons are CAA (vs. 25%), 81% of Glu codons are GAA (vs. 40%), 94% of Cys codons are UGU (vs. 42%), only 1% of Ser codons are UCU (vs. 24%), CCC is never used for Phe and UAG is never used as a stop codon. Thus protein-coding genes can be made more like genes which nature has already optimised for expression in canine cells, thereby facilitating expression.

Reverse Genetics

Reverse genetics for influenza viruses can be practised with 12 expression constructs to express the four proteins required to initiate replication and transcription (PB1, PB2, PA and NP) and all eight viral genome segments. To reduce the number of expression constructs, however, a plurality of RNA polymerase I transcription cassettes (for viral RNA synthesis) can be included on a single expression construct (e.g. sequences encoding 1, 2, 3, 4, 5, 6, 7 or all 8 influenza vRNA segments), and a plurality of protein-coding regions with RNA polymerase II promoters on another expression construct (e.g. sequences encoding 1, 2, 3, 4, 5, 6, 7 or 8 influenza mRNA transcripts) [17]. It is also possible to include one or more influenza vRNA segments under control of a pol I promoter and one or more influenza protein coding regions under control of another promoter, in particular a pol II promoter, on the same expression construct. This is preferably done by using bi-directional expression constructs.

Known reverse genetics systems involve expressing viral RNA (vRNA) molecules from pol I promoters, bacterial RNA polymerase promoters, bacteriophage polymerase promoters, etc. As influenza viruses require the presence of viral polymerase to initiate the life cycle, systems may also provide these proteins e.g. the system further comprises expression constructs that encode viral polymerase proteins such that expression of both types of DNA leads to assembly of a complete infectious virus. It is also possible to supply the viral polymerase as a protein.

Where reverse genetics is used for the expression of influenza vRNA, it will be evident to the person skilled in the art that precise spacing of the sequence elements with reference to each other is important for the polymerase to initiate replication. It is therefore important that the sequence encoding the viral RNA is positioned correctly between the pol I promoter and the termination sequence, but this positioning is well within the capabilities of those who work with reverse genetics systems.

In order to produce a recombinant virus, a cell must express all segments of the viral genome which are necessary to assemble a virion. The expression constructs preferably provide all of the viral RNA and proteins, but it is also possible to use a helper virus to provide some of the RNA and proteins, although systems which do not use a helper virus are preferred.

In some embodiments an expression construct will also be included which leads to expression of an accessory protein in the host cell. For instance, it can be advantageous to express a non-viral serine protease (e.g. trypsin) as part of a reverse genetics system.

Viral Segments

The synthetic expression construct encodes one or more viral segments. During the early days of an influenza pandemic it is not unusual to have sequences of the circulating strains available which include only the complete coding region but incomplete untranslated regions (UTRs). Awaiting the complete segment sequence (including the coding region and the UTRs) before commencing production of viruses costs time and delays the provision of the vaccines. The inventors have provided an improved method for preparing a synthetic expression construct encoding a viral segment, which method reduces the time required to obtain the viral segment. The method comprises the steps of: (a) providing the sequence of at least part of the coding region of the HA or NA segment from an influenza virus; (b) identifying the HA and/or NA subtype of the virus from which the coding region is derived; (c) providing a UTR sequence from an influenza virus with the same HA or NA subtype as the subtype identified in step (b); and (d) preparing a synthetic expression construct which encodes a viral segment comprising the coding sequence and the UTR.

The sequence of the coding region of the viral segment can be provided by sequencing the circulating strain. The sequence may also be obtained from other sources such as, for example, a health care authority. Preferably, the whole coding region is used in the method as this will facilitate the determination of the HA or NA subtype of the virus from which the coding region is derived. It is also possible to use at least part of the coding region provided the coding region is complete enough to allow the determination of the HA or NA subtype. This will generally be the case where a fragment covering at least 90%, at least 95%, or at least 99% of the full-length coding region is available. The viral segment used in the analysis is preferably the HA or NA segment.

The HA and/or NA subtype of the virus from which the coding sequence is derived can be determined using standard methods in the art. For example, the sequence of the coding region can be aligned to the sequences of coding regions from viruses with known HA and/or NA subtypes. The coding regions which are aligned need, of course, be the coding region of the same viral segment (e.g. the HA or NA segment). Influenza viral segments from viruses with the same HA and/or NA subtype will show the highest sequence identity between the sequences. Suitable programs for performing the analysis are known in the art and include BLAST™.

In order to provide a suitable UTR for the viral segment, the UTR of the viral strain which showed the highest sequence identity in step (a) can be used. Alternatively, the UTR can be identified by determining the consensus sequences of UTRs from viral strains with the same HA or NA subtype. This can be achieved by aligning two or more influenza strains with the same HA or NA subtype and determining the conserved residues in the UTRs. For example, the consensus sequence may be determined by aligning the UTRs from 2, 5, 10, 15, 20, 30 or more influenza strains with the same HA or NA subtype. The consensus UTR sequence can then be used to prepare the complete DNA molecule. Suitable programs for aligning multiple sequences are known in the art and include ClustalW2™.

Where the DNA molecules are prepared using a consensus UTR sequence, it is not necessary to determine this consensus sequence every time. Instead, the analysis can be performed for influenza virus strains with various HA and NA subtypes and the resulting UTRs for each HA and NA subtype can be kept in a database. Once the HA or NA subtype of the circulating strain has been determined it is then necessary only to choose the UTR of an influenza strain with the same HA or NA subtype from the database.

The DNA molecule comprising the coding sequence and the identified UTRs can be prepared by any of the methods described herein.

The Culture Host

The influenza viruses are typically produced using a cell line, although primary cells may be used as an alternative. The cell will typically be mammalian, although avian or insect cells can also be used. Suitable mammalian cells include, but are not limited to, human, hamster, cattle, primate and dog cells. In some embodiments, the cell is a human non-kidney cell or a non-human cell. Various cells may be used, such as kidney cells, fibroblasts, retinal cells, lung cells, etc. Examples of suitable hamster cells are the cell lines having the names BHK21 or HKCC. Suitable monkey cells are e.g. African green monkey cells, such as kidney cells as in the Vero cell line [18-20]. Suitable dog cells are e.g. kidney cells, as in the CLDK and MDCK cell lines. Suitable avian cells include the EBx cell line derived from chicken embryonic stem cells, EB45, EB14, and EB14-074 [21].

Further suitable cells include, but are not limited to: CHO; MRC 5; PER.C6 [22]; FRhL2; WI-38; etc. Suitable cells are widely available e.g. from the American Type Cell Culture (ATCC) collection [23], from the Coriell Cell Repositories [24], or from the European Collection of Cell Cultures (ECACC). For example, the ATCC supplies various different Vero cells under catalogue numbers CCL 81, CCL 81.2, CRL 1586 and CRL-1587, and it supplies MDCK cells under catalogue number CCL 34. PER.C6 is available from the ECACC under deposit number 96022940.

Preferred cells for use in the invention are MDCK cells [25-27], derived from Madin Darby canine kidney. The original MDCK cells are available from the ATCC as CCL 34. It is preferred that derivatives of these or other MDCK cells are used. Such derivatives were described, for instance, in reference 25 which discloses MDCK cells that were adapted for growth in suspension culture (‘MDCK 33016’ or ‘33016-PF’, deposited as DSM ACC 2219). Furthermore, reference 28 discloses MDCK-derived cells that grow in suspension in serum free culture (‘B-702’, deposited as FERM BP-7449). In some embodiments, the MDCK cell line used may be tumorigenic, but it is also envisioned to use non-tumorigenic MDCK cells. For example, reference 29 discloses non-tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (ATCC PTA-6503). Reference 30 discloses MDCK cells with high susceptibility to infection, including ‘MDCK.5F1’ cells (ATCC CRL 12042).

It is possible to use a mixture of more than one cell type in the methods of the invention, but it is preferred to use a single cell type e.g. using monoclonal cells. Where a mixture of cells is used, it is preferred that the mixture does not contain 293T cells as these cells are not approved for vaccine manufacture.

The cells used in the methods of the invention are preferably cells which are suitable for producing an influenza vaccine that can be used for administration to humans. Such cells must be derived from a cell bank system which is approved for vaccine manufacture and registered with a national control authority, and must be within the maximum number of passages permitted for vaccine production (see reference 31 for a summary). Examples of suitable cells which have been approved for vaccine manufacture include MDCK cells (like MDCK 33016; see reference 25), CHO cells, Vero cells, and PER.C6 cells. The methods of the invention preferably do not use 293T cells as these cells are not approved for vaccine manufacture.

Preferably, the cells used for preparing the virus and for preparing the vaccine are of the same cell type. For example, the cells may both be MDCK, Vero or PerC6 cells. This is preferred because it facilitates regulatory approval as approval needs to be obtained only for a single cell line. It also has the further advantage that competing culture selection pressures or different cell culture conditions can be avoided. The methods of the invention may also use the same cell line throughout, for example MDCK 33016.

The influenza viruses prepared according to the methods of the invention may subsequently be propagated in eggs. The current standard method for influenza virus growth for vaccines uses embryonated SPF hen eggs, with virus being purified from the egg contents (allantoic fluid). It is also possible to passage a virus through eggs and subsequently propagate it in cell culture and vice versa.

Preferably, the cells are cultured in the absence of serum, to avoid a common source of contaminants. Various serum-free media for eukaryotic cell culture are known to the person skilled in the art e.g. Iscove's medium, ultra CHO medium (BioWhittaker), EX-CELL (JRH Biosciences). Furthermore, protein-free media may be used e.g. PF-CHO (JRH Biosciences). Otherwise, the cells for replication can also be cultured in the customary serum-containing media (e.g. MEM or DMEM medium with 0.5% to 10% of fetal calf serum).

The cells may be in adherent culture or in suspension.

Reassortant Viruses

The reassortant influenza strains produced by the methods of the invention contain viral segments from a vaccine strain and one or more donor strain(s). The vaccine strain is the influenza strain which provides the HA segment of the reassortant influenza strain. The vaccine strain can be any strain and can vary from season to season.

A donor strain is an influenza strain which provides one or more of the backbone segments (i.e. those encoding PB1, PB2, PA, NP, M₁, M₂, NS₁ and NS₂) of the influenza strain. The NA segment may also be provided by a donor strain or it may be provided by the vaccine strain. The reassortant influenza strains of the invention may also comprise one or more, but not all, of the backbone segments from the vaccine strain. As the reassortant influenza virus contains a total of eight segments, it will therefore contain x (wherein x is from 1-7) viral segments from the vaccine strain and 8-x viral segments from the one or more donor strain(s).

The reassortant influenza virus strains may grow to higher or similar viral titres in cell culture and/or in eggs in the same time (for example 12 hours, 24 hours, 48 hours or 72 hours) and under the same growth conditions compared to the wild-type vaccine strain. In particular, they can grow to higher or similar viral titres in MDCK cells (such as MDCK 33016) in the same time and under the same growth conditions compared to the wild-type vaccine strain. The viral titre can be determined by standard methods known to those of skill in the art. Usefully, the reassortant viruses of the invention may achieve a viral titre which is at least 5% higher, at least 10% higher, at least 20% higher, at least 50% higher, at least 100% higher, at least 200% higher, or at least 500% higher than the viral titre of the wild-type vaccine strain in the same time frame and under the same conditions. The reassortant influenza viruses may also grow to similar viral titres in the same time and under the same growth conditions compared to the wild-type vaccine strain. A similar titre in this context means that the reassortant influenza viruses grow to a titre which is within 3% of the viral titre achieved with the wild-type vaccine strain in the same time and under the same growth conditions (i.e. wild-type titre±3%).

The reassortant viruses of the invention can contain the backbone segments from two or more donor strains, or at least one (i.e. one, two, three, four, five or six) backbone viral segment from a donor strain as described herein. The backbone viral segments are those which do not encode HA or NA. Thus, backbone segments will typically encode the PB1, PB2, PA, NP, M₁, M₂, NS₁ and NS₂ polypeptides of the influenza virus.

When the reassortant viruses of the invention are reassortants comprising the backbone segments from a single donor strain, the reassortant viruses will generally include segments from the donor strain and the vaccine strain in a ratio of 1:7, 2:6, 3:5, 4:4, 5:3, 6:2 or 7:1. Having a majority of segments from the donor strain, in particular a ratio of 6:2, is typical. When the reassortant viruses comprise backbone segments from two donor strains, the reassortant virus will generally include segments from the first donor strain, the seconds donor strain and the vaccine strain in a ratio of 1:1:6, 1:2:5, 1:3:4, 1:4:3, 1:5:2, 1:6:1, 2:1:5, 2:2:4, 2:3:3, 2:4:2, 2:5:1, 3:1:2, 3:2:1, 4:1:3, 4:2:2, 4:3:1, 5:1:2, 5:2:1 or 6:1:1. The reassortant influenza viruses may also comprise viral segments from more than two, for example from three, four, five or six donor strains.

Where the reassortant influenza virus comprises backbone segments from two or three donor strains, each donor strain may provide more than one of the backbone segments of the reassortant influenza virus, but one or two of the donor strains can also provide only a single backbone segment.

Where the reassortant influenza virus comprises backbone segments from two, three, four or five donor strains, one or two of the donor strains may provide more than one of the backbone segments of the reassortant influenza virus. In general the reassortant influenza virus cannot comprise more than six backbone segments. Accordingly, for example, if one of the donor strains provides five of the viral segments, the reassortant influenza virus can only comprise backbone segments from a total of two different donor strains.

In general a reassortant influenza virus will contain only one of each backbone segment. For example, when the influenza virus comprises the NP segment from B/Brisbane/60/08 it will not at the same time comprise the NP segment from another influenza strain.

Strains which can be used as vaccine strains include strains which are resistant to antiviral therapy (e.g. resistant to oseltamivir [32] and/or zanamivir), including resistant pandemic strains [33].

The reassortant influenza strains produced by the methods of the invention may comprise segments from a vaccine strain which is an inter-pandemic (seasonal) influenza vaccine strain. It may also comprise segments from a vaccine strain which is a pandemic strain or a potentially pandemic strain. The characteristics of an influenza strain that give it the potential to cause a pandemic outbreak are: (a) it contains a new hemagglutinin compared to the hemagglutinins in currently-circulating human strains, i.e. one that has not been evident in the human population for over a decade (e.g. H2), or has not previously been seen at all in the human population (e.g. H5, H6 or H9, that have generally been found only in bird populations), such that the human population will be immunologically naive to the strain's hemagglutinin; (b) it is capable of being transmitted horizontally in the human population; and (c) it is pathogenic to humans. A vaccine strain with H5 hemagglutinin type is preferred where the reassortant virus is used in vaccines for immunizing against pandemic influenza, such as a H5N1 strain. Other possible strains include H5N3, H9N2, H2N2, H7N1 and H7N7, and any other emerging potentially pandemic strains. The invention is particularly suitable for producing reassortant viruses for use in vaccine for protecting against potential pandemic virus strains that can or have spread from a non-human animal population to humans, for example a swine-origin H1N1 influenza strain.

The methods of the invention can be used to prepare reassortant influenza A strains and reassortant influenza B strains.

Reassortant Influenza A Viruses

Where the methods are used to prepare reassortant influenza A strains, the strains may contain the influenza A virus HA subtypes H1, H2, H3, H4, H5, H6, H7, H8, H9, H10, H11, H12, H13, H14, H15, H16 or H17. They may contain the influenza A virus NA subtypes N1, N2, N3, N4, N5, N6, N7, N8 or N9. Where the vaccine strain is a seasonal influenza strain, it may have a H1 or H3 subtype. In one aspect of the invention the vaccine strain is a H1N1 or H3N2 strain.

The reassortant influenza A viruses preferably comprise at least one backbone viral segment from the donor strain PR8-X. Thus, the influenza viruses of the invention may comprise one or more genome segments selected from: a PA segment having at least 95%, at least 96%, at least 97%, at least 98%, at least 99% or 100% identity to the sequence of SEQ ID NO: 9, a PB1 segment having at least 95%, at least 96%, at least 97%, at least 98%, at least 99% or 100% identity to the sequence of SEQ ID NO: 10, a PB2 segment having at least 95%, at least 96%, at least 97%, at least 98%, at least 99% or 100% identity to the sequence of SEQ ID NO: 11, a M segment having at least 95%, at least 96%, at least 97%, at least 98%, at least 99% or 100% identity to the sequence of SEQ ID NO: 13, a NP segment having at least 95%, at least 96%, at least 97%, at least 98%, at least 99% or 100% identity to the sequence of SEQ ID NO: 12, and/or a NS segment having at least 95%, at least 96%, at least 97%, at least 98%, at least 99% or 100% identity to the sequence of SEQ ID NO: 14. The reassortant influenza A virus may comprise all of these backbone segments.

Alternatively, or in addition, the reassortant influenza A virus may comprise one or more backbone viral segments from the 105p30 strain. Thus, where the reassortant influenza A virus comprises one or more genome segments from the 105p30 strain, the viral segments may have sequences selected from: a PA segment having at least 95%, at least 96%, at least 97%, at least 98%, at least 99% or 100% identity to the sequence of SEQ ID NO: 42, a PB1 segment having at least 95%, at least 96%, at least 97%, at least 98%, at least 99% or 100% identity to the sequence of SEQ ID NO: 43, a PB2 segment having at least 95%, at least 96%, at least 97%, at least 98%, at least 99% or 100% identity to the sequence of SEQ ID NO: 44, a M segment having at least 95%, at least 96%, at least 97%, at least 98%, at least 99% or 100% identity to the sequence of SEQ ID NO: 46, a NP segment having at least 95%, at least 96%, at least 97%, at least 98%, at least 99% or 100% identity to the sequence of SEQ ID NO: 45, and/or a NS segment having at least 95%, at least 96%, at least 97%, at least 98%, at least 99% or 100% identity to the sequence of SEQ ID NO: 47. The reassortant influenza A virus may comprise all of these backbone segments.

The reassortant influenza viruses may comprise backbone segments from two or more influenza donor strains. The inventors have found that such reassortant influenza A viruses grow particularly well in culture hosts. For example, the inventors have found that a reassortant influenza A virus comprising the NP, PB1 and PB2 segments from 105p30 and the M, NS and PA segments from PR8-X provided a higher rescue efficiency and grew faster compared to reassortant influenza A viruses which comprise all backbone segments from PR8-X. Likewise, a reassortant influenza A strain comprising the PB1 segment from A/California/4/09 and the other backbone segments from PR8-X often had greater rescue efficiencies and HA yields than reassortant influenza A viruses which comprise all backbone segments from PR8-X. Such reassortant influenza A viruses are particularly suitable for use in the methods of the invention because the increased rescue efficiency increases the speed further by which seed viruses for vaccine manufacture can be obtained.

Reassortant influenza A viruses with backbone segments from two or more influenza donor strains may comprise the HA segment and the PB1 segment from different influenza A strains. In these reassortant influenza viruses the PB1 segment may be from donor viruses with the same influenza virus HA subtype as the vaccine strain. For example, the PB1 segment and the HA segment may both be from influenza viruses with a H1 subtype. The reassortant influenza A viruses may also comprise the HA segment and the PB1 segment from different influenza A strains with different influenza virus HA subtypes, wherein the PB1 segment is not from an influenza virus with a H3 HA subtype and/or wherein the HA segment is not from an influenza virus with a H1 or H5 HA subtype. For example, the PB1 segment may be from a H1 virus and/or the HA segment may be from a H3 influenza virus. Where the reassortants contain viral segments from more than one influenza donor strain, the further donor strain(s) can be any donor strain. For example, some of the viral segments may be derived from the A/Puerto Rico/8/34 or A/Ann Arbor/6/60 influenza strains. Reassortants containing viral segments from the A/Ann Arbor/6/60 strain may be advantageous, for example, where the reassortant virus is to be used in a live attenuated influenza vaccine.

The reassortant influenza A virus may also comprise backbone segments from two or more influenza donor strains, wherein the PB1 segment is from the A/California/07/09 influenza strain. This segment may have at least 95% identity, at least 96% identity, at least 97% identity, at least 98% identity, at least 99% identity or 100% identity with the sequence of SEQ ID NO: 24. The reassortant influenza A virus may have the H1 HA subtype. It will be understood that a reassortant influenza virus according to this aspect of the invention will not comprise the HA and/or NA segments from A/California/07/09.

The reassortant influenza strains may comprise the HA segment and/or the NA segment from an A/California/4/09 strain. Thus, for instance, the HA gene segment may encode a H1 hemagglutinin which is more closely related to SEQ ID NO: 70 than to SEQ ID NO: 50 (i.e. has a higher degree sequence identity when compared to SEQ ID NO: 70 than to SEQ ID NO: 50 using the same algorithm and parameters). SEQ ID NOs: 70 and 50 are 80% identical. Similarly, the NA gene may encode a N1 neuraminidase which is more closely related to SEQ ID NO: 99 than to SEQ ID NO: 51. SEQ ID NOs: 99 and 51 are 82% identical.

The reassortant influenza A virus may also comprise at least one backbone viral segment from the A/California/07/09 influenza strain. When the at least one backbone viral segment is the PA segment it may have a sequence having at least 95%, at least 96%, at least 97% or at least 99% identity with the sequence of SEQ ID NO: 23. When the at least one backbone viral segment is the PB1 segment, it may have a sequence having at least 95%, at least 96%, at least 97% or at least 99% identity with the sequence of SEQ ID NO: 24. When the at least one backbone viral segment is the PB2 segment, it may have a sequence having at least 95%, at least 96%, at least 97% or at least 99% identity with the sequence of SEQ ID NO: 25. When the at least one backbone viral segment is the NP segment it may have a sequence having at least 95%, at least 96%, at least 97% or at least 99% identity with the sequence of SEQ ID NO: 26. When the at least one backbone viral segment is the M segment it may have a sequence having at least 95%, at least 96%, at least 97% or at least 99% identity with the sequence of SEQ ID NO: 27. When the at least one backbone viral segment is the NS segment it may have a sequence having at least 95%, at least 96%, at least 97% or at least 99% identity with the sequence of SEQ ID NO: 28.

Where a reassortant influenza A virus comprises the PB1 segment from A/Texas/1/77, it preferably does not comprise the PA, NP or M segment from A/Puerto Rico/8/34. Where a reassortant influenza A virus comprises the PA, NP or M segment from A/Puerto Rico/8/34, it preferably does not comprise the PB1 segment from A/Texas/1/77. In some embodiments, the invention does not encompass reassortant influenza A viruses which have the PB1 segment from A/Texas/1/77 and the PA, NP and M segments from A/Puerto Rico/8/34. The PB1 protein from A/Texas/1/77 may have the sequence of SEQ ID NO: 29 and the PA, NP or M proteins from A/Puerto Rico/8/34 may have the sequence of SEQ ID NOs 30, 31 or 32, respectively.

The backbone viral segments may be optimized for culture in the specific culture host. For example, where the reassortant influenza viruses are cultured in mammalian cells, it is advantageous to adapt at least one of the viral segments for optimal growth in the culture host. For example, where the expression host is a canine cell, such as a MDCK cell line, the viral segments may have a sequence which optimises viral growth in the cell. Thus, the reassortant influenza viruses of the invention may comprise a PB2 genome segment which has lysine in the position corresponding to amino acid 389 of SEQ ID NO: 3 when aligned to SEQ ID NO: 3 using a pairwise alignment algorithm, and/or asparagine in the position corresponding to amino acid 559 of SEQ ID NO: 3 when aligned to SEQ ID NO: 3 using a pairwise alignment algorithm. Also provided are reassortant influenza viruses in accordance with the invention in which the PA genome segment has lysine in the position corresponding to amino acid 327 of SEQ ID NO: 1 when aligned to SEQ ID NO: 1 using a pairwise alignment algorithm, and/or aspartic acid in the position corresponding to amino acid 444 of SEQ ID NO: 1 when aligned to SEQ ID NO: 1, using a pairwise alignment algorithm, and/or aspartic acid in the position corresponding to amino acid 675 of SEQ ID NO: 1 when aligned to SEQ ID NO: 1, using a pairwise alignment algorithm. The reassortant influenza strains of the invention may also have a NP genome segment with threonine in the position corresponding to amino acid 27 of SEQ ID NO: 4 when aligned to SEQ ID NO: 4 using a pairwise alignment algorithm, and/or asparagine in the position corresponding to amino acid 375 of SEQ ID NO: 4 when aligned to SEQ ID NO: 4, using a pairwise alignment algorithm. Variant influenza strains may also comprise two or more of these mutations. It is preferred that the variant influenza virus contains a variant PB2 segment with both of the amino acids changes identified above, and/or a PA which contains all three of the amino acid changes identified above, and/or a NP segment which contains both of the amino acid changes identified above. The influenza A virus may be a H1 strain.

Alternatively, or in addition, the reassortant influenza A viruses may comprise a PB1 segment which has isoleucine in the position corresponding to amino acid 200 of SEQ ID NO: 2 when aligned to SEQ ID NO: 2 using a pairwise alignment algorithm, and/or asparagine in the position corresponding to amino acid 338 of SEQ ID NO: 2 when aligned to SEQ ID NO: 2 using a pairwise alignment algorithm, and/or isoleucine in the position corresponding to amino acid 529 of SEQ ID NO: 2 when aligned to SEQ ID NO: 2 using a pairwise alignment algorithm, and/or isoleucine in the position corresponding to amino acid 591 of SEQ ID NO: 2 when aligned to SEQ ID NO: 2 using a pairwise alignment algorithm, and/or histidine in the position corresponding to amino acid 687 of SEQ ID NO: 2 when aligned to SEQ ID NO: 2 using a pairwise alignment algorithm, and/or lysine in the position corresponding to amino acid 754 of SEQ ID NO: 2 when aligned to SEQ ID NO: 2 using a pairwise alignment algorithm.

The preferred pairwise alignment algorithm is the Needleman-Wunsch global alignment algorithm [34], using default parameters (e.g. with Gap opening penalty=10.0, and with Gap extension penalty=0.5, using the EBLOSUM62 scoring matrix). This algorithm is conveniently implemented in the needle tool in the EMBOSS package [35].

The choice of donor strain for use in the methods of the invention can depend on the vaccine strain which is to be reassorted. As reassortants between evolutionary distant strains might not replicate well in cell culture, it is possible that the donor strain and the vaccine strain have the same HA and/or NA subtype. In other embodiments, however, the vaccine strain and the donor strain can have different HA and/or NA subtypes, and this arrangement can facilitate selection for reassortant viruses that contain the HA and/or NA segment from the vaccine strain. Therefore, although the 105p30 and PR8-X strains contain the H1 influenza subtype these donor strains can be used for vaccine strains which do not contain the H1 influenza subtype.

Reassortants of the donor strains wherein the HA and/or NA segment has been changed to another subtype can also be used. The H1 influenza subtype of the 105p30 or PR8-X strain may be changed, for example, to a H3 or H5 subtype.

Thus, an influenza A virus may comprises one, two, three, four, five, six or seven viral segments from the 105p30 or PR8-X strains and a HA segment which is not of the H1 subtype. The reassortant donor strains may further comprise an NA segment which is not of the N1 subtype.

The reassortant donor strains may comprise at least one, at least two, at least three, at least four, at least five, at least six or at least seven viral segments from the 105p30 or PR8-X strains of the invention and a H1 HA segment which is derived from a different influenza strain.

The ‘second influenza strain’ used in the methods of the invention is different to the donor strain which is used.

Reassortant Influenza B Viruses

The invention can also be used to prepare reassortant influenza B strains.

For example, the methods can be used to produce a reassortant influenza B virus which comprises the HA segment from a first influenza B virus and the NP and/or PB2 segment from a second influenza B virus which is a BNictoria/2/87-like strain. The BNictoria/2/87-like strain may be B/Brisbane/60/08.

The methods can also be used to produce reassortant influenza B viruses comprising the HA segment from a first influenza B virus and the NP segment from a second influenza B virus which is not B/Lee/40 or B/Ann Arbor/1/66 or B/Panama/45/90. For example, the reassortant influenza B virus may have a NP segment which does not have the sequence of SEQ ID NOs: 80, 100, 103 or 104. The reassortant influenza B virus may also have a NP segment which does not encode the protein of SEQ ID NOs: 19, 23, 44 or 45. The reassortant influenza B virus may comprise both the NP and PB2 segments from the second influenza B virus. The second influenza B virus is preferably a B/Victoria/2/87-like strain. The B/Victoria/2/87-like strain may be B/Brisbane/60/08.

The invention can also be used to produce a reassortant influenza B virus comprising the HA segment from a B/Yamagata/16/88-like strain and at least one backbone segment from a B/Victoria/2/87-like strain. The reassortant influenza B virus may comprise two, three, four, five or six backbone segments from the B/Victoria/2/87-like strain. In a preferred embodiment, the reassortant influenza B virus comprises all the backbone segments from the BNictoria/2/87-like strain. The B/Victoria/2/87-like strain may be B/Brisbane/60/08.

The methods are also suitable for producing a reassortant influenza B virus comprising viral segments from a B/Victoria/2/87-like strain and a B/Yamagata/16/88-like strain, wherein the ratio of segments from the B/Victoria/2/87-like strain and the B/Yamagata/16/88-like strain is 1:7, 2:6, 4:4, 5:3, 6:2 or 7:1. A ratio of 7:1, 6:2, 4:4, 3:4 or 1:7, in particular a ratio of 4:4, is preferred because such reassortant influenza B viruses grow particularly well in a culture host. The BNictoria/2/87-like strain may be B/Brisbane/60/08. The B/Yamagata/16/88-like strain may be B/Panama/45/90. In these embodiments, the reassortant influenza B virus usually does not comprise all backbone segments from the same influenza B donor strain.

The methods can also be used to produce a reassortant influenza B virus which comprises:

a) the PA segment of SEQ ID NO: 71, the PB1 segment of SEQ ID NO: 72, the PB2 segment of SEQ ID NO: 73, the NP segment of SEQ ID NO: 74, the NS segment of SEQ ID NO: 76 and the M segment of SEQ ID NO: 75; or

b) the PA segment of SEQ ID NO: 71, the PB1 segment of SEQ ID NO: 78, the PB2 segment of SEQ ID NO: 73, the NP segment of SEQ ID NO: 74, the NS segment of SEQ ID NO: 82 and the M segment of SEQ ID NO: 81; or

c) the PA segment of SEQ ID NO: 71, the PB1 segment of SEQ ID NO: 78, the PB2 segment of SEQ ID NO: 79, the NP segment of SEQ ID NO: 74, the NS segment of SEQ ID NO: 76 and the M segment of SEQ ID NO: 75; or

d) the PA segment of SEQ ID NO: 30, the PB1 segment of SEQ ID NO: 72, the PB2 segment of SEQ ID NO: 73, the NP segment of SEQ ID NO: 74, the NS segment of SEQ ID NO: 76 and the M segment of SEQ ID NO: 75, or

e) the PA segment of SEQ ID NO: 71, the PB1 segment of SEQ ID NO: 72, the PB2 segment of SEQ ID NO: 73, the NP segment of SEQ ID NO: 74, the NS segment of SEQ ID NO: 82 and the M segment of SEQ ID NO: 81.

Influenza B viruses currently do not display different HA subtypes, but influenza B virus strains do fall into two distinct lineages. These lineages emerged in the late 1980s and have HAs which can be antigenically and/or genetically distinguished from each other [36]. Current influenza B virus strains are either BNictoria/2/87-like or B/Yamagata/16/88-like. These strains are usually distinguished antigenically, but differences in amino acid sequences have also been described for distinguishing the two lineages e.g. B/Yamagata/16/88-like strains often (but not always) have HA proteins with deletions at amino acid residue 164, numbered relative to the ‘Lee40’ HA sequence [37]. In some embodiments, the reassortant influenza B viruses of the invention may comprise viral segments from a BNictoria/2/87-like strain. They may comprise viral segments from a B/Yamagata/16/88-like strain. Alternatively, they may comprise viral segments from a B/Victoria/2/87-like strain and a B/Yamagata/16/88-like strain.

Where the reassortant influenza B virus comprises viral segments from two or more influenza B virus strains, these viral segments may be derived from influenza strains which have related neuraminidases. For instance, the influenza strains which provide the viral segments may both have a B/Victoria/2/87-like neuraminidase [38] or may both have a B/Yamagata/16/88-like neuraminidase. For example, two BNictoria/2/87-like neuraminidases may both have one or more of the following sequence characteristics: (1) not a serine at residue 27, but preferably a leucine; (2) not a glutamate at residue 44, but preferably a lysine; (3) not a threonine at residue 46, but preferably an isoleucine; (4) not a proline at residue 51, but preferably a serine; (5) not an arginine at residue 65, but preferably a histidine; (6) not a glycine at residue 70, but preferably a glutamate; (7) not a leucine at residue 73, but preferably a phenylalanine; and/or (8) not a proline at residue 88, but preferably a glutamine. Similarly, in some embodiments the neuraminidase may have a deletion at residue 43, or it may have a threonine; a deletion at residue 43, arising from a trinucleotide deletion in the NA gene, which has been reported as a characteristic of B/Victoria/2/87-like strains, although recent strains have regained Thr-43 [38]. Conversely, of course, the opposite characteristics may be shared by two B/Yamagata/16/88-like neuraminidases e.g. S27, E44, T46, P51, R65, G70, L73, and/or P88. These amino acids are numbered relative to the ‘Lee40’ neuraminidase sequence [39]. The reassortant influenza B virus may comprise a NA segment with the characteristics described above.

Alternatively, or in addition, the reassortant influenza B virus may comprise a viral segment (other than NA) from an influenza strain with a NA segment with the characteristics described above.

The backbone viral segments of an influenza B virus which is a B/Victoria/2/87-like strain can have a higher level of identity to the corresponding viral segment from B/Victoria/2/87 than it does to the corresponding viral segment of B/Yamagata/16/88 and vice versa. For example, the NP segment of B/Panama/45/90 (which is a B/Yamagata/16/88-like strain) has 99% identity to the NP segment of B/Yamagata/16/88 and only 96% identity to the NP segment of BNictoria/2/87.

Where the reassortant influenza B virus of the invention comprises a backbone viral segment from a B/Victoria/2/87-like strain, the viral segments may encode proteins with the following sequences. The PA protein may have at least 97% identity, at least 98%, at least 99% identity or 100% identity to the sequence of SEQ ID NO: 83. The PB1 protein may have at least 97% identity, at least 98%, at least 99% identity or 100% identity to the sequence of SEQ ID NO: 84. The PB2 protein may have at least 97%, at least 98%, at least 99% or 100% identity with the sequence of SEQ ID NO: 85. The NP protein may have at least 97% identity, at least 98%, at least 99% identity or 100% identity to the sequence of SEQ ID NO: 86. The M₁ protein may have at least 97% identity, at least 98%, at least 99% identity or 100% identity to the sequence of SEQ ID NO: 87. The M₂ protein may have at least 97% identity, at least 98%, at least 99% identity or 100% identity to the sequence of SEQ ID NO: 88. The NS₁ protein may have at least 97% identity, at least 98%, at least 99% identity or 100% identity to the sequence of SEQ ID NO: 89. The NS₂ protein may have at least 97% identity, at least 98%, at least 99% identity or 100% identity to the sequence of SEQ ID NO: 90. In some embodiments, the reassortant influenza B virus may also comprise all of these backbone segments.

Where the reassortant influenza B viruses of the invention comprise a backbone viral segment from a B/Yamagata/16/88-like strain, the viral segment may encode proteins with the following sequences. The PA protein may have at least 97% identity, at least 98%, at least 99% identity or 100% identity to the sequence of SEQ ID NO: 91. The PB1 protein may have at least 97% identity, at least 98%, at least 99% identity or 100% identity to the sequence of SEQ ID NO: 92. The PB2 protein may have at least 97%, at least 98%, at least 99% or 100% identity with the sequence of SEQ ID NO: 93. The NP protein may have at least 97% identity, at least 98%, at least 99% identity or 100% identity to the sequence of SEQ ID NO: 94. The M₁ protein may have at least 97% identity, at least 98%, at least 99% identity or 100% identity to the sequence of SEQ ID NO: 95. The M₂ protein may have at least 97% identity, at least 98%, at least 99% identity or 100% identity to the sequence of SEQ ID NO: 96. The NS₁ protein may have at least 97% identity, at least 98%, at least 99% identity or 100% identity to the sequence of SEQ ID NO: 97. The NS₂ protein may have at least 97% identity, at least 98%, at least 99% identity or 100% identity to the sequence of SEQ ID NO: 98.

The invention can be practised with donor strains having a viral segment that has at least about 70%, at least about 75%, at least about 80%, at least about 85%, at least about 90%, at least about 95% or at least about 99%, or 100% identity to a sequence of SEQ ID NOs 71-76 or 77-82. Due to the degeneracy of the genetic code, it is possible to have the same polypeptide encoded by several nucleic acids with different sequences. For example, the nucleic acid sequences of SEQ ID NOs: 33 and 34 have only 73% identity even though they encode the same viral protein. Thus, the invention may be practised with viral segments that encode the same polypeptides as the sequences of SEQ ID NOs 71-76 or 77-82.

Reassortant viruses which contain an NS segment that does not encode a functional NS protein are also within the scope of the present invention. NS1 knockout mutants are described in reference 40. These NS1-mutant virus strains are particularly suitable for preparing live attenuated influenza vaccines.

The ‘second influenza strain’ used in the methods of the invention is different to the donor strain which is used.

Backbone Libraries

In order to supply influenza vaccines rapidly during a pandemic it is important that the reassortant influenza viruses can grow to high viral titres in a short time frame. The inventors have discovered that it can be useful to test a number of reassortant influenza viruses comprising the HA and NA segments of the vaccine strain in combination with different backbones in order to identify the fastest growing reassortants. The invention thus provides a library comprising two or more influenza backbones. For example, the library may comprise 5, 10, 15, 20, 30, 40, 50, 100 or 200 different influenza backbones. The backbones may be included on expression constructs in the library. In some embodiments, the library may not comprise expression constructs which encode the HA and/or NA segments of influenza viruses as these segments will come from the circulating influenza strain. The library may comprise at least one influenza backbone as described in the preceding sections.

Each expression construct in the library may encode all the backbone segments of an influenza virus. It is also possible to include expression constructs which do not encode all the backbone segments. For example, the library may comprise expression constructs which encode one, two, three, four, five, six or seven viral backbone segment(s).

When a new circulating strain is identified, the HA and NA segments of that strain may be included in an expression construct (which may be a synthetic expression construct). This expression construct and the expression constructs in the library can be co-transfected into host cells (which are preferably all of the same cell line or the same cell type). Cells which receive expression constructs that encode all the viral segments of an influenza virus will produce reassortant influenza viruses from these expression constructs. In this manner, it is possible to produce a number of different reassortant influenza viruses which all comprise the same HA and NA segments but which will have different backbone segments. The growth rate of these reassortant influenza viruses can be determined using standard methods in the art and the fastest growing reassortant can be selected for vaccine production.

Virus Preparation

In one embodiment, the invention provides a method for producing influenza viruses comprising steps of (a) infecting a culture host with a reassortant virus of the invention; (b) culturing the host from step (a) to produce the virus; and optionally (c) purifying the virus produced in step (b).

The culture host may be cells or embryonated hen eggs, as described above. Where cells are used as a culture host in this aspect of the invention, it is known that cell culture conditions (e.g. temperature, cell density, pH value, etc.) are variable over a wide range subject to the cell line and the virus employed and can be adapted to the requirements of the application. The following information therefore merely represents guidelines.

As mentioned above, cells are preferably cultured in serum-free or protein-free media.

Multiplication of the cells can be conducted in accordance with methods known to those of skill in the art. For example, the cells can be cultivated in a perfusion system using ordinary support methods like centrifugation or filtration. Moreover, the cells can be multiplied according to the invention in a fed-batch system before infection. In the context of the present invention, a culture system is referred to as a fed-batch system in which the cells are initially cultured in a batch system and depletion of nutrients (or part of the nutrients) in the medium is compensated by controlled feeding of concentrated nutrients. It can be advantageous to adjust the pH value of the medium during multiplication of cells before infection to a value between pH 6.6 and pH 7.8 and especially between a value between pH 7.2 and pH 7.3. Culturing of cells preferably occurs at a temperature between 30 and 40° C. When culturing the infected cells (step b), the cells are preferably cultured at a temperature of between 30° C. and 36° C. or between 32° C. and 34° C. or at 33° C. This is particularly preferred, as it has been shown that incubation of infected cells in this temperature range results in production of a virus that results in improved efficacy when formulated into a vaccine [41].

Oxygen partial pressure can be adjusted during culturing before infection preferably at a value between 25% and 95% and especially at a value between 35% and 60%. The values for the oxygen partial pressure stated in the context of the invention are based on saturation of air. Infection of cells occurs at a cell density of preferably about 8-25×10⁵ cells/mL in the batch system or preferably about 5-20×10⁶ cells/mL in the perfusion system. The cells can be infected with a viral dose (MOI value, “multiplicity of infection”; corresponds to the number of virus units per cell at the time of infection) between 10⁻⁸ and 10, preferably between 0.0001 and 0.5.

Virus may be grown on cells in adherent culture or in suspension. Microcarrier cultures can be used. In some embodiments, the cells may thus be adapted for growth in suspension.

The methods according to the invention also include harvesting and isolation of viruses or the proteins generated by them. During isolation of viruses or proteins, the cells are separated from the culture medium by standard methods like separation, filtration or ultrafiltration. The viruses or the proteins are then concentrated according to methods sufficiently known to those skilled in the art, like gradient centrifugation, filtration, precipitation, chromatography, etc., and then purified. It is also preferred according to the invention that the viruses are inactivated during or after purification. Virus inactivation can occur, for example, by β-propiolactone or formaldehyde at any point within the purification process.

The culture host may be eggs. The current standard method for influenza virus growth for vaccines uses embryonated SPF hen eggs, with virus being purified from the egg contents (allantoic fluid). It is also possible to passage a virus through eggs and subsequently propagate it in cell culture and vice versa.

Vaccine

The invention utilises virus produced according to the method to produce vaccines.

Vaccines (particularly for influenza virus) are generally based either on live virus or on inactivated virus. Inactivated vaccines may be based on whole virions, split virions, or on purified surface antigens. Antigens can also be presented in the form of virosomes. The invention can be used for manufacturing any of these types of vaccine.

Where an inactivated virus is used, the vaccine may comprise whole virion, split virion, or purified surface antigens (for influenza, including hemagglutinin and, usually, also including neuraminidase). Chemical means for inactivating a virus include treatment with an effective amount of one or more of the following agents: detergents, formaldehyde, β-propiolactone, methylene blue, psoralen, carboxyfullerene (C60), binary ethylamine, acetyl ethyleneimine, or combinations thereof. Non-chemical methods of viral inactivation are known in the art, such as for example UV light or gamma irradiation.

Virions can be harvested from virus-containing fluids, e.g. allantoic fluid or cell culture supernatant, by various methods. For example, a purification process may involve zonal centrifugation using a linear sucrose gradient solution that includes detergent to disrupt the virions. Antigens may then be purified, after optional dilution, by diafiltration.

Split virions are obtained by treating purified virions with detergents (e.g. ethyl ether, polysorbate 80, deoxycholate, tri-N-butyl phosphate, Triton X-100, Triton N101, cetyltrimethylammonium bromide, Tergitol NP9, etc.) to produce subvirion preparations, including the ‘Tween-ether’ splitting process. Methods of splitting influenza viruses, for example are well known in the art e.g. see refs. 42-47, etc. Splitting of the virus is typically carried out by disrupting or fragmenting whole virus, whether infectious or non-infectious with a disrupting concentration of a splitting agent. The disruption results in a full or partial solubilisation of the virus proteins, altering the integrity of the virus. Preferred splitting agents are non-ionic and ionic (e.g. cationic) surfactants e.g. alkylglycosides, alkylthioglycosides, acyl sugars, sulphobetaines, betains, polyoxyethylenealkylethers, N,N-dialkyl-Glucamides, Hecameg, alkylphenoxy-polyethoxyethanols, NP9, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl- or nonylphenoxy polyoxyethanols (e.g. the Triton surfactants, such as Triton X-100 or Triton N101), polyoxyethylene sorbitan esters (the Tween surfactants), polyoxyethylene ethers, polyoxyethlene esters, etc. One useful splitting procedure uses the consecutive effects of sodium deoxycholate and formaldehyde, and splitting can take place during initial virion purification (e.g. in a sucrose density gradient solution). Thus a splitting process can involve clarification of the virion-containing material (to remove non-virion material), concentration of the harvested virions (e.g. using an adsorption method, such as CaHPO₄ adsorption), separation of whole virions from non-virion material, splitting of virions using a splitting agent in a density gradient centrifugation step (e.g. using a sucrose gradient that contains a splitting agent such as sodium deoxycholate), and then filtration (e.g. ultrafiltration) to remove undesired materials. Split virions can usefully be resuspended in sodium phosphate-buffered isotonic sodium chloride solution. Examples of split influenza vaccines are the BEGRIVAC™, FLUARIX™, FLUZONE™ and FLUSHIELD™ products.

Purified influenza virus surface antigen vaccines comprise the surface antigens hemagglutinin and, typically, also neuraminidase. Processes for preparing these proteins in purified form are well known in the art. The FLUVIRIN™, AGRIPPAL™ and INFLUVAC™ products are influenza subunit vaccines.

Another form of inactivated antigen is the virosome [48] (nucleic acid free viral-like liposomal particles). Virosomes can be prepared by solubilization of virus with a detergent followed by removal of the nucleocapsid and reconstitution of the membrane containing the viral glycoproteins. An alternative method for preparing virosomes involves adding viral membrane glycoproteins to excess amounts of phospholipids, to give liposomes with viral proteins in their membrane.

The methods of the invention may also be used to produce live vaccines. Such vaccines are usually prepared by purifying virions from virion-containing fluids. For example, the fluids may be clarified by centrifugation, and stabilized with buffer (e.g. containing sucrose, potassium phosphate, and monosodium glutamate). Various forms of influenza virus vaccine are currently available (e.g. see chapters 17 & 18 of reference 49). Live virus vaccines include MedImmune's FLUMIST™ product (trivalent live virus vaccine).

The virus may be attenuated. The virus may be temperature-sensitive. The virus may be cold-adapted. These three features are particularly useful when using live virus as an antigen.

HA is the main immunogen in current inactivated influenza vaccines, and vaccine doses are standardised by reference to HA levels, typically measured by SRID. Existing vaccines typically contain about 15 μg of HA per strain, although lower doses can be used e.g. for children, or in pandemic situations, or when using an adjuvant. Fractional doses such as ½ (i.e. 7.5 μg HA per strain), ¼ and ⅛ have been used, as have higher doses (e.g. 3× or 9× doses [50,51]). Thus vaccines may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5 μg, etc. Particular doses include e.g. about 45, about 30, about 15, about 10, about 7.5, about 5, about 3.8, about 3.75, about 1.9, about 1.5, etc. per strain.

For live vaccines, dosing is measured by median tissue culture infectious dose (TCID₅₀) rather than HA content, and a TCID₅₀ of between 10⁶ and 10⁸ (preferably between 10⁶⁵-10⁷⁵) per strain is typical.

Influenza strains used with the invention may have a natural HA as found in a wild-type virus, or a modified HA. For instance, it is known to modify HA to remove determinants (e.g. hyper-basic regions around the HA1/HA2 cleavage site) that cause a virus to be highly pathogenic in avian species. The use of reverse genetics facilitates such modifications.

As well as being suitable for immunizing against inter-pandemic strains, the compositions of the invention are particularly useful for immunizing against pandemic or potentially-pandemic strains. The invention is suitable for vaccinating humans as well as non-human animals.

Other strains whose antigens can usefully be included in the compositions are strains which are resistant to antiviral therapy (e.g. resistant to oseltamivir [52] and/or zanamivir), including resistant pandemic strains [53].

Compositions of the invention may include antigen(s) from one or more (e.g. 1, 2, 3, 4 or more) influenza virus strains, including influenza A virus and/or influenza B virus provided that at least one influenza strain is a reassortant influenza strain of the invention. Compositions wherein at least two, at least three or all of the antigens are from reassortant influenza strains of the invention are also envisioned. Where a vaccine includes more than one strain of influenza, the different strains are typically grown separately and are mixed after the viruses have been harvested and antigens have been prepared. Thus a process of the invention may include the step of mixing antigens from more than one influenza strain. A trivalent vaccine is typical, including antigens from two influenza A virus strains and one influenza B virus strain. A tetravalent vaccine is also useful [54], including antigens from two influenza A virus strains and two influenza B virus strains, or three influenza A virus strains and one influenza B virus strain.

Pharmaceutical Compositions

Vaccine compositions manufactured according to the invention are pharmaceutically acceptable. They usually include components in addition to the antigens e.g. they typically include one or more pharmaceutical carrier(s) and/or excipient(s). As described below, adjuvants may also be included. A thorough discussion of such components is available in reference 55.

Vaccine compositions will generally be in aqueous form. However, some vaccines may be in dry form, e.g. in the form of injectable solids or dried or polymerized preparations on a patch.

Vaccine compositions may include preservatives such as thiomersal or 2-phenoxyethanol. It is preferred, however, that the vaccine should be substantially free from (i.e. less than 5 μg/ml) mercurial material e.g. thiomersal-free [46,56]. Vaccines containing no mercury are more preferred. An α-tocopherol succinate can be included as an alternative to mercurial compounds [46]. Preservative-free vaccines are particularly preferred.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, etc.

Vaccine compositions will generally have an osmolality of between 200 mOsm/kg and 400 mOsm/kg, preferably between 240-360 mOsm/kg, and will more preferably fall within the range of 290-310 mOsm/kg. Osmolality has previously been reported not to have an impact on pain caused by vaccination [57], but keeping osmolality in this range is nevertheless preferred.

Vaccine compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a vaccine composition will generally be between 5.0 and 8.1, and more typically between 6.0 and 8.0 e.g. 6.5 and 7.5, or between 7.0 and 7.8. A process of the invention may therefore include a step of adjusting the pH of the bulk vaccine prior to packaging.

The vaccine composition is preferably sterile. The vaccine composition is preferably non-pyrogenic e.g. containing <1 EU (endotoxin unit, a standard measure) per dose, and preferably <0.1 EU per dose. The vaccine composition is preferably gluten-free.

Vaccine compositions of the invention may include detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate, particularly for a split or surface antigen vaccine. The detergent may be present only at trace amounts. Thus the vaccine may include less than 1 mg/ml of each of octoxynol-10 and polysorbate 80. Other residual components in trace amounts could be antibiotics (e.g. neomycin, kanamycin, polymyxin B).

A vaccine composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Influenza vaccines are typically administered in a dosage volume of about 0.5 ml, although a half dose (i.e. about 0.25 ml) may be administered to children.

Compositions and kits are preferably stored at between 2° C. and 8° C. They should not be frozen. They should ideally be kept out of direct light.

Host Cell DNA

Where virus has been isolated and/or grown on a cell line, it is standard practice to minimize the amount of residual cell line DNA in the final vaccine, in order to minimize any potential oncogenic activity of the DNA.

Thus a vaccine composition prepared according to the invention preferably contains less than 10 ng (preferably less than ing, and more preferably less than 100 pg) of residual host cell DNA per dose, although trace amounts of host cell DNA may be present.

It is preferred that the average length of any residual host cell DNA is less than 500 bp e.g. less than 400 bp, less than 300 bp, less than 200 bp, less than 100 bp, etc.

Contaminating DNA can be removed during vaccine preparation using standard purification procedures e.g. chromatography, etc. Removal of residual host cell DNA can be enhanced by nuclease treatment e.g. by using a DNase. A convenient method for reducing host cell DNA contamination is disclosed in references 58 & 59, involving a two-step treatment, first using a DNase (e.g. Benzonase), which may be used during viral growth, and then a cationic detergent (e.g. CTAB), which may be used during virion disruption. Treatment with an alkylating agent, such as β-propiolactone, can also be used to remove host cell DNA, and advantageously may also be used to inactivate virions [60].

Adjuvants

Compositions of the invention may advantageously include an adjuvant, which can function to enhance the immune responses (humoral and/or cellular) elicited in a subject who receives the composition. Preferred adjuvants comprise oil-in-water emulsions. Various such adjuvants are known, and they typically include at least one oil and at least one surfactant, with the oil(s) and surfactant(s) being biodegradable (metabolisable) and biocompatible. The oil droplets in the emulsion are generally less than 5 μm in diameter, and ideally have a sub-micron diameter, with these small sizes being achieved with a microfluidiser to provide stable emulsions. Droplets with a size less than 220 nm are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Another preferred oil is α-tocopherol (see below).

Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Where the vaccine contains a split virus, it is preferred that it contains free surfactant in the aqueous phase. This is advantageous as the free surfactant can exert a ‘splitting effect’ on the antigen, thereby disrupting any unsplit virions and/or virion aggregates that might otherwise be present. This can improve the safety of split virus vaccines [61].

Preferred emulsions have an average droplets size of <1 μm, e.g. ≤750 nm, ≤500 nm, ≤400 nm, ≤300 nm, ≤250 nm, ≤220 nm, ≤200 nm, or smaller These droplet sizes can conveniently be achieved by techniques such as microfluidisation.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [62-64], as described in more detail in Chapter 10 of ref. 65 and chapter 12 of ref. 66. The MF59 emulsion advantageously includes citrate ions e.g. 10 mM sodium citrate buffer.
An emulsion comprising squalene, a tocopherol, and polysorbate 80. The emulsion may include phosphate buffered saline. These emulsions may have by volume from 2 to 10% squalene, from 2 to 10% tocopherol and from 0.3 to 3% polysorbate 80, and the weight ratio of squalene:tocopherol is preferably <1 (e.g. 0.90) as this can provide a more stable emulsion. Squalene and polysorbate 80 may be present in a volume ratio of about 5:2 or at a weight ratio of about 11:5. Thus the three components (squalene, tocopherol, polysorbate 80) may be present at a weight ratio of 1068:1186:485 or around 55:61:25. One such emulsion (‘AS03’) can be made by dissolving Tween 80 in PBS to give a 2% solution, then mixing 90 ml of this solution with a mixture of (5 g of DL a tocopherol and 5 ml squalene), then microfluidising the mixture. The resulting emulsion may have submicron oil droplets e.g. with an average diameter of between 100 and 250 nm, preferably about 180 nm. The emulsion may also include a 3-de-O-acylated monophosphoryl lipid A (3d MPL). Another useful emulsion of this type may comprise, per human dose, 0.5-10 mg squalene, 0.5-11 mg tocopherol, and 0.1-4 mg polysorbate 80 [67] e.g. in the ratios discussed above.
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [68] (0.05-1% Thr-MDP, 5% squalane, 2.5% Pluronic L121 and 0.2% polysorbate 80). It can also be used without the Thr-MDP, as in the “AF” adjuvant [69] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [70]. The emulsion may also include one or more of: alditol; a cryoprotective agent (e.g. a sugar, such as dodecylmaltoside and/or sucrose); and/or an alkylpolyglycoside. The emulsion may include a TLR4 agonist [71]. Such emulsions may be lyophilized
An emulsion of squalene, poloxamer 105 and Abil-Care [72]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 73, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 74, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.
An emulsion in which a saponin (e.g. QuilA or QS21) and a sterol (e.g. a cholesterol) are associated as helical micelles [75].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [76].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [76].

In some embodiments an emulsion may be mixed with antigen extemporaneously, at the time of delivery, and thus the adjuvant and antigen may be kept separately in a packaged or distributed vaccine, ready for final formulation at the time of use. In other embodiments an emulsion is mixed with antigen during manufacture, and thus the composition is packaged in a liquid adjuvanted form.

The antigen will generally be in an aqueous form, such that the vaccine is finally prepared by mixing two liquids. The volume ratio of the two liquids for mixing can vary (e.g. between 5:1 and 1:5) but is generally about 1:1 and this is most preferred. Where concentrations of components are given in the above descriptions of specific emulsions, these concentrations are typically for an undiluted composition, and the concentration after mixing with an antigen solution will thus decrease (e.g. it will be half the concentration where the antigen and the adjuvant are mixed at a ratio of 1:1).

Packaging of Vaccine Compositions

Suitable containers for compositions of the invention (or kit components) include vials, syringes (e.g. disposable syringes), nasal sprays, etc. These containers should be sterile.

Where a composition/component is located in a vial, the vial is preferably made of a glass or plastic material. The vial is preferably sterilized before the composition is added to it. To avoid problems with latex-sensitive patients, vials are preferably sealed with a latex-free stopper, and the absence of latex in all packaging material is preferred. The vial may include a single dose of vaccine, or it may include more than one dose (a ‘multidose’ vial) e.g. 10 doses. Preferred vials are made of colourless glass.

A vial can have a cap (e.g. a Luer lock) adapted such that a pre-filled syringe can be inserted into the cap, the contents of the syringe can be expelled into the vial (e.g. to reconstitute lyophilised material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed. A vial may have a cap that permits aseptic removal of its contents, particularly for multidose vials.

Where a component is packaged into a syringe, the syringe may have a needle attached to it. If a needle is not attached, a separate needle may be supplied with the syringe for assembly and use. Such a needle may be sheathed. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of a butyl rubber. If the syringe and needle are packaged separately then the needle is preferably fitted with a butyl rubber shield. Preferred syringes are those marketed under the trade name “Tip-Lok”™.

Containers may be marked to show a half-dose volume e.g. to facilitate delivery to children. For instance, a syringe containing a 0.5 ml dose may have a mark showing a 0.25 ml volume.

Where a glass container (e.g. a syringe or a vial) is used, then it is preferred to use a container made from a borosilicate glass rather than from a soda lime glass.

A kit or composition may be packaged (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

Methods of Treatment, and Administration of the Vaccine

The invention provides a vaccine manufactured according to the invention. These vaccine compositions are suitable for administration to human or non-human animal subjects, such as pigs or birds, and the invention provides a method of raising an immune response in a subject, comprising the step of administering a composition of the invention to the subject. The invention also provides a composition of the invention for use as a medicament, and provides the use of a composition of the invention for the manufacture of a medicament for raising an immune response in a subject.

The immune response raised by these methods and uses will generally include an antibody response, preferably a protective antibody response. Methods for assessing antibody responses, neutralising capability and protection after influenza virus vaccination are well known in the art. Human studies have shown that antibody titers against hemagglutinin of human influenza virus are correlated with protection (a serum sample hemagglutination-inhibition titer of about 30-40 gives around 50% protection from infection by a homologous virus) [77]. Antibody responses are typically measured by hemagglutination inhibition, by microneutralisation, by single radial immunodiffusion (SRID), and/or by single radial hemolysis (SRH). These assay techniques are well known in the art.

Compositions of the invention can be administered in various ways. The most preferred immunisation route is by intramuscular injection (e.g. into the arm or leg), but other available routes include subcutaneous injection, intranasal [78-80], oral [81], intradermal [82,83], transcutaneous, transdermal [84], etc.

Vaccines prepared according to the invention may be used to treat both children and adults. Influenza vaccines are currently recommended for use in pediatric and adult immunisation, from the age of 6 months. Thus a human subject may be less than 1 year old, 1-5 years old, 5-15 years old, 15-55 years old, or at least 55 years old. Preferred subjects for receiving the vaccines are the elderly (e.g. ≥50 years old, ≥60 years old, and preferably ≥65 years), the young (e.g. ≤5 years old), hospitalised subjects, healthcare workers, armed service and military personnel, pregnant women, the chronically ill, immunodeficient subjects, subjects who have taken an antiviral compound (e.g. an oseltamivir or zanamivir compound; see below) in the 7 days prior to receiving the vaccine, people with egg allergies and people travelling abroad. The vaccines are not suitable solely for these groups, however, and may be used more generally in a population. For pandemic strains, administration to all age groups is preferred.

Preferred compositions of the invention satisfy 1, 2 or 3 of the CPMP criteria for efficacy. In adults (18-60 years), these criteria are: (1) ≥70% seroprotection; (2) ≥40% seroconversion; and/or (3) a GMT increase of ≥2.5-fold. In elderly (>60 years), these criteria are: (1) ≥60% seroprotection; (2) ≥30% seroconversion; and/or (3) a GMT increase of ≥2-fold. These criteria are based on open label studies with at least 50 patients.

Treatment can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Administration of more than one dose (typically two doses) is particularly useful in immunologically naive patients e.g. for people who have never received an influenza vaccine before, or for vaccinating against a new HA subtype (as in a pandemic outbreak). Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H.influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, a pneumococcal conjugate vaccine, etc. Administration at substantially the same time as a pneumococcal vaccine and/or a meningococcal vaccine is particularly useful in elderly patients.

Similarly, vaccines of the invention may be administered to patients at substantially the same time as (e.g. during the same medical consultation or visit to a healthcare professional) an antiviral compound, and in particular an antiviral compound active against influenza virus (e.g. oseltamivir and/or zanamivir). These antivirals include neuraminidase inhibitors, such as a (3R,4R,5S)-4-acetylamino-5-amino-3(1-ethylpropoxy)-1-cyclohexene-1-carboxylic acid or 5-(acetylamino)-4-[(aminoiminomethyl)-amino]-2,6-anhydro-3,4,5-trideoxy-D-glycero-D-galactonon-2-enonic acid, including esters thereof (e.g. the ethyl esters) and salts thereof (e.g. the phosphate salts). A preferred antiviral is (3R,4R,5S)-4-acetylamino-5-amino-3(1-ethylpropoxy)-1-cyclohexene-1-carboxylic acid, ethyl ester, phosphate (1:1), also known as oseltamivir phosphate (TAMIFLU™).

Other Biologicals

Whilst the invention has been described with reference to influenza viruses and influenza vaccines, the invention can also be used for the production of other viruses which can be produced by reverse genetics, as well as other viral vaccines. For example, the methods of the invention are particularly suitable for producing viruses such as dengue virus, rotaviruses, measles virus, rubella virus, coronaviruses.

Other biologicals which can be produced recombinantly can also be produced by the methods of the invention. Suitable examples include antibodies, growth factors, cytokines, lymphokines, receptors, hormones, diagnostic antigens, etc.

The method steps described herein will apply mutatis mutandis to these viruses, vaccines or biologicals.

General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

The various steps of the methods may be carried out at the same or different times, in the same or different geographical locations, e.g. countries, and by the same or different people or entities.

Where animal (and particularly bovine) materials are used in the culture of cells, they should be obtained from sources that are free from transmissible spongiform encephalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of reference 85. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is taught in reference 86.

References to a percentage sequence identity between two nucleic acid sequences mean that, when aligned, that percentage of bases are the same in comparing the two sequences. This alignment and the percent homology or sequence identity can be determined using software programs known in the art, for example those described in section 7.7.18 of reference 85. A preferred alignment program is GCG Gap (Genetics Computer Group, Wisconsin, Suite Version 10.1), preferably using default parameters, which are as follows: open gap=3; extend gap=1.

### BRIEF DESCRIPTION OF THE DRAWINGS

FIG. 1(A), FIG. 1(B), and FIG. 1(C). Method of synthetic gene segment assembly and error correction. FIG. 1(A) Process flow. Time for performance of each step is indicated on the right. FIG. 1(B) Schematic diagram of process. “X” indicates sites of oligonucleotide synthesis errors. In the circular DNA and final assembled gene diagrams (the bottom two), pKS10 sequences are white, and influenza coding sequences are black. FIG. 1(C) Ethidium bromide stained agarose gel of linear synthetic HA and NA genes, including regulatory elements used for virus rescue. MW—molecular weight marker.

FIG. 2. Timeline of rescue of synthetic H7N9 influenza viruses from transmission of oligonucleotide sequence information to confirmation of recovered viruses.

FIG. 3(A), FIG. 3(B), and FIG. 3(C). Performance of synthetic H7N9 reassortant viruses from the simulated pandemic response. FIG. 3(A) Titers of influenza viruses in culture fluid harvested from MDCK-supplemented 293T cells 48 hours (dotted columns) and 72 hours (white columns) after co-transfection with the indicated backbone plasmids and synthetic HA and NA gene constructs. Viral titers were determined by a focus formation assay using MDCK cell monolayers. FIG. 3(B) Replication kinetics of synthetic H7N9 reassortant viruses in MDCK 33016PF suspension cultures. FIG. 3(C) HA yields from synthetic H7N9 viruses in MDCK suspension cultures, determined by RP-HPLC after purification of viruses on sucrose density gradients. The y-axis in FIG. 3(A) and FIG. 3(B) shows infectious units (log10 IU/mL). The y-axis in FIG. 3(C) shows HA yield in μg/mL.

FIG. 4(A) and FIG. 4(B). Effect of MDCK feeder cell addition 24 hours after transfection of MDCK cells on rescue efficiency. Titers of recombinant viruses containing the PR8× backbone with HA and NA segments from either FIG. 4(A) A/WSN/1933 (H1N1) or FIG. 4(B) A/California/04/2009 were measured 72 hours after transfection by a focus formation assay. The dotted column shows the results with additional cells whilst the white column shows the results without additional cells. The y-axis indicates infectious units (log10 IU/mL).

FIG. 5(A), FIG. 5(B), FIG. 5(C), FIG. 5(D), FIG. 5(E), and FIG. 5(F). Synthetic influenza virus rescue efficiencies. Representative data showing effect of optimized backbones on virus rescue efficiency from transfected cultures of MDCK cells. Detection of influenza viruses in culture fluid harvested at different time points after transfection with the indicated backbone plasmids and synthetic HA and NA constructs, or 24-48 hours after a blind passage using 500 μl of the culture fluid on fresh MDCK cell monolayers (Passage 1). Viral titers were determined using a focus formation assay for FIG. 5(A) an H1N1 strain, FIG. 5(B) an H3N2 strain, FIG. 5(C) an attenuated H5N1 strain, FIG. 5(D) a swine origin H3N2v strain, FIG. 5(E) a B/Yamagata lineage strain, and FIG. 5(F) a B/Victoria lineage strain. The y-axis indicates infectious units (log10 IU/mL).

FIG. 6. Rescue of synthetic H7N9a viruses from either MDCK-supplemented 293T cells or from MDCK cells only. Detection of influenza viruses in culture fluid harvested 48 hours (dotted columns) and 72 hours (white columns) after transfection with the #19 backbone plasmids and synthetic H7 and N9 constructs. Viral titers were determined on MDCK cell monolayers using a focus formation assay. The y-axis indicates infectious units (log10 IU/mL).

FIG. 7(A), FIG. 7(B), and FIG. 7(C). Replication kinetics of synthetic H7N9 reassortant viruses with alternative NA UTRs in MDCK 33016PF suspension cultures. Replication kinetics of synthetic H7N9 viruses with alternative NA UTRs and different backbones, FIG. 7(A) PR8x, FIG. 7(B) #19, and FIG. 7(C) #21, in MDCK suspension cultures. Starting m.o.i. was 0.001. The x-axis indicates the hours post infection. The y-axis indicates infectious units (log10 IU/mL).

FIG. 8. HA yield by turkey RBC agglutination by synthetic H7N9 viruses with alternative NA UTRs. The y-axis indicates the HA units.

FIG. 9(A) and FIG. 9(B) compares the HA content (determined by lectin-capture ELISA) of sucrose gradient-purified viruses harvested at 60 h post-infection from MDCK cell cultures infected with reverse genetics-derived 6:2 reassortants containing either the PR8-X or #21 backbone with the HA and NA segments from FIG. 9(A) a pandemic-like H1 strain (strain 1) or FIG. 9(B) a second pandemic-like strain (strain 2). In FIG. 9(A) and FIG. 9(B), the black bar represents a reference vaccine strain (derived from WHO-Collaborating Centre-supplied strain) as control, the grey bar represents a reassortant virus containing the PR8-X backbone, and the white bar represents a reassortant virus containing the #21 backbone. The y-axis indicates HA yield in μg/ml.

FIG. 10(A) and FIG. 10(B) compares the HA content (determined by a lectin-capture ELISA) of unpurified viruses harvested at 60 h post-infection from MDCK cell cultures infected with reverse genetics-derived 6:2 reassortants containing either the PR8-X or #21 backbone with the HA and NA segments from FIG. 10(A) a pre-pandemic H1 strain (strain 1) and FIG. 10(B) a second pre-pandemic H1 strain (strain 2). In FIG. 10(A) and FIG. 10(B), the black bar represents a reference vaccine strain (derived from WHO-Collaborating Centre-supplied strain) as control, the grey bar represents a reassortant virus containing the PR8-X backbone, and the white bar represents a reassortant virus containing the #21 backbone. The y-axis indicates HA yield in μg/ml.

FIG. 11 compares the HA yield (determined by HPLC) of sucrose-purified viruses harvested at 60 h post-infection from MDCK cell cultures infected with reverse genetics-derived 6:2 reassortants containing either the PR8-X or #21 backbone with the HA and NA segments from an H3 strain (strain 1). The black bar represents a reference vaccine strain (derived from WHO-Collaborating Centre-supplied strain) as control, the grey bar represents a reassortant virus containing the PR8-X backbone, and the white bar represents a reassortant virus containing the #21 backbone. The y-axis indicates HA yield in μg/ml.

FIG. 12(A) and FIG. 12(B) compares virus titers (determined by focus formation assay (FFA); FIG. 12(A)) and HA titers (determined by lectin-capture ELISA; FIG. 12(B)) of viruses harvested from embyronated chicken eggs at 60 h post-infection with a reference vaccine strain or reverse genetics-derived 6:2 reassortant viruses made with either the PR8-X or #21 backbone and the HA and NA segments from a pandemic-like H1 strain (strain 2). In FIG. 12(A), the individual dots represent data from single eggs. The line represents the average of the individual data points. The y-axis indicates infectious units/ml. In FIG. 12(B), the black bar represents the reference vaccine strain (derived from WHO-Collaborating Centre-supplied strain), the grey bar represents a reassortant virus containing the PR8-X backbone, and the white bar represents a reassortant virus containing the #21 backbone. The y-axis indicates HA yield in μg/ml for pooled egg samples.

FIG. 13 compares the HA yield of different reassortant influenza B strains in MDCK cells relative to the wild-type (WT) or reverse genetics-derived (RG) B/Brisbane/60/08 strain. The viral segments of the tested influenza B viruses are shown in Table 1. The y-axis indicates the HA yield in μg/mL.

FIG. 14 compares the HA yield of different reassortant influenza B strains in MDCK cells relative to the wild-type (WT) or reverse genetics-derived (RG) B/Panama/45/90 strain. The viral segments of the tested influenza B viruses are shown in Table 1. The y-axis indicates the HA yield in μg/mL.

### MODES FOR CARRYING OUT THE INVENTION

Increased Gene Synthesis Speed and Accuracy through Enzymatic Assembly and In Vitro Error Correction.

A purely enzymatic one-step, isothermal assembly method of gene assembly, previously used to synthesize the entire 16,299 base pair mouse mitochondrial genome from 600 overlapping 60-base oligonucleotides (6), was adapted for the generation of synthetic DNA copies of influenza virus genome segments. The method uses 5′ T5 exonuclease (Epicentre), Phusion DNA polymerase (New England Biolabs [NEB]) and Taq DNA ligase (NEB) to join multiple DNA fragments during a brief 50° C. reaction (7). The method was selected to assemble genes for synthetic vaccine seeds because it is rapid and readily automated. All bases of the resulting synthetic genes have their origin in chemically synthesized oligonucleotides. Using current techniques, DNA oligonucleotide synthesis has an error rate of about 1 per 325 bases, typically due to missing bases from failed chemical coupling, and the error rate increases with the length of the oligonucleotide synthesized (6). When DNA copies of the 1.7 kb HA and 1.5 kb NA viral RNA genome segments are synthesized by this technique using oligonucleotides approximately 60 bases in length with 30 bases of overlap between oligonucleotides on opposite strands, only 3% of the synthetic products have the correct sequence. During the mouse mitochondrial genome synthesis, subassemblies were cloned and sequenced, and sets of error-free sequences were selected for subsequent rounds of assembly (6). For the purpose of rapid influenza vaccine seed virus generation, this method of error correction would introduce unacceptable delays.

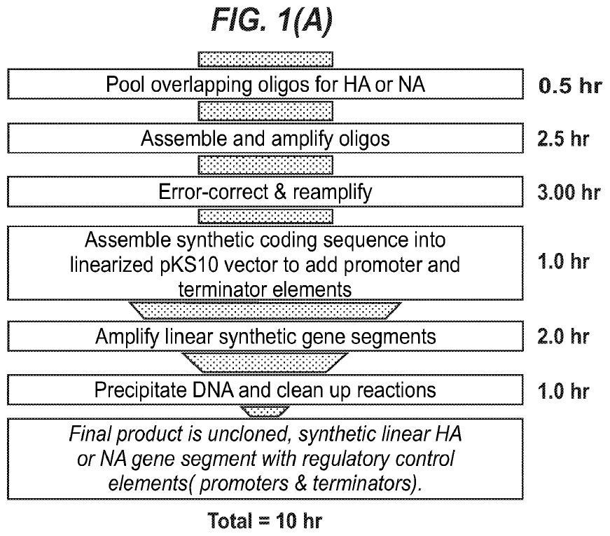

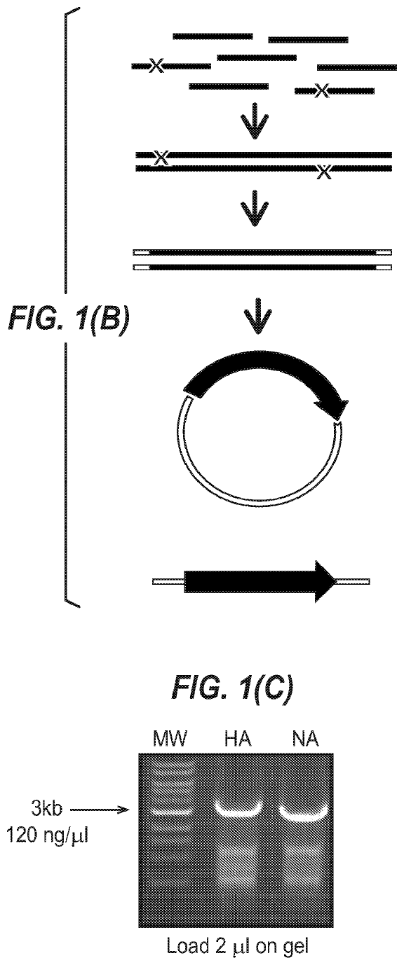

The problem of synthesizing DNA copies of HA and NA genome segments with both accuracy and speed was solved by (i) increasing the overlap between oligonucleotides, (ii) introducing an enzymatic error correction step, and (iii) increasing the number of oligonucleotides assembled at once, eliminating the need for stepwise assembly via sub-assemblies (FIG. 1(A) and FIG. 1(B)). Specifically, the length of oligonucleotides was increased to 60-74 bases, and full length genes (including 5′ and 3′ un-translated regions) were assembled from staggered sets of oligonucleotides that contained all residues of a double-stranded DNA molecule so that, prior to ligation, the full double-stranded gene can be annealed. In practice, a software algorithm generates a set of sequences for oligonucleotides (a maximum of 96 oligonucleotides per HA, NA pair) that meet these criteria. After chemical synthesis of the oligonucleotides, enzymatic isothermal assembly, and PCR amplification, error-containing DNA is removed enzymatically by treating melted and re-annealed DNA with the commercially available ErrASE error correction kit (Novici Biotech), which excises areas of base mismatch in double-stranded DNA molecules before another round of PCR amplification.

After agarose gel verification of the products' sizes, the control sequences (including Pol I and Pol II promoters and their terminator and polyadenylation signals) needed to generate RNA genome segments and mRNA for virus rescue are added by isothermally coupling the synthetic DNA with a linearized plasmid (pKS10) that contains these regulatory sequences (87). Nucleotide identity between the ends of the linearized plasmid and the 5′ and 3′ primers used for gene synthesis guide this assembly. The assembled molecule is the substrate for a round of high fidelity PCR amplification using primers outside the transcription control regions.

After purification and concentration of the amplicons, approximately 10 μg of assembled linear DNA cassettes that contain the influenza gene flanked by control sequences are obtained, ready for transfection into the MDCK 33016PF cell line for influenza virus rescue (FIG. 1(C)). The time from receipt of oligonucleotides to a purified HA or NA-encoding DNA cassette ready for transfection is approximately 10 hours. While virus rescue is underway using the enzymatically assembled, error corrected, and amplified DNA, parallel cloning and sequencing verifies the sequence of the assembled genes. Typically, 80-100% of the full-length sequences obtained are correct.

Optimized Rescue of Influenza Viruses from Synthetic DNA on a Vaccine Manufacturing Cell Line.

The rescue protocol for synthetic seed virus generation is adapted from a previously described eight-plasmid ambisense system in which each expression plasmid has a cDNA copy of a viral gene segment bounded at the 5′ end by a Pol II promoter to drive transcription of messenger RNA and at the 3′ end by a human Pol I promoter to drive transcription of negative-stranded influenza RNA genome segments (88). The manufacturing-qualified MDCK 33016PF cell line is a less efficient substrate for transfection and influenza virus rescue by reverse genetics than 293T cells (which are not qualified for vaccine production). Influenza virus reverse genetic rescue has been described using Vero cells (some banks of which are qualified for vaccine production) (89, 90). However, using one cell line for vaccine virus rescue and a different cell line for antigen production would add adventitious agent risk and regulatory and manufacturing complexity. Therefore, we elected to increase the efficiency of reverse genetic DNA rescue in MDCK 33016PF cells so that a single cell line can be used for seed generation and vaccine antigen production. Although Pol I promoters are generally species specific, human Pol I efficiently drives transcription in MDCK 33016PF cells, which are of canine origin.

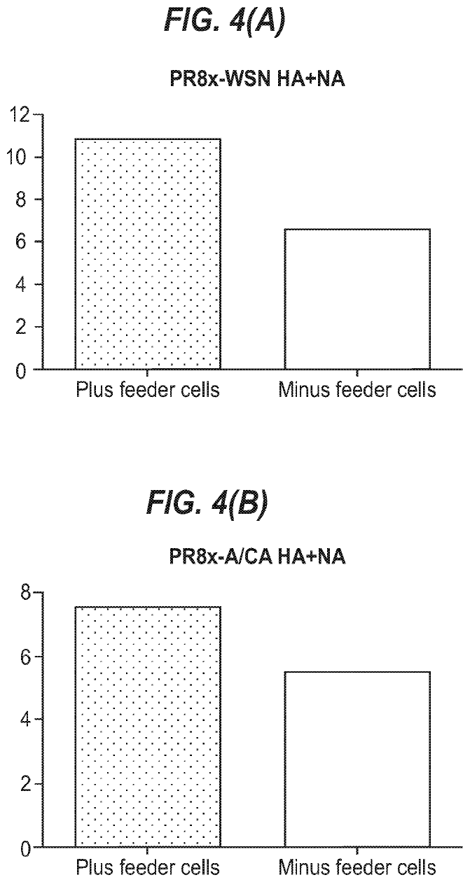

One μg of each linear synthetic cassette encoding HA or NA is co-transfected into MDCK 33016PF cells together with 1 μg of each ambisense plasmid that encodes PA, PB1, PB2, NP, NS, or M and a helper plasmid that encodes the protease TMPRSS2 (91). To increase rescue efficiency, we add cultures of fresh (un-transfected) MDCK 33016PF cells after transfection, which increases the probability of virus recovery, presumably by providing a healthier population of cells in which rescued viruses can further amplify (FIG. 4(A) and FIG. 4(B)). Viruses are detected in cell culture medium within 72 hours after transfection (approximately 24 hours later than after transfection of Vero or 293T cells), using a focus-formation assay in which the medium from the transfected culture is added to a fresh MDCK cell monolayer, and infectious virus is detected by immuno-staining for expressed NP.

Improved Backbones for Synthetic Virus Rescue.

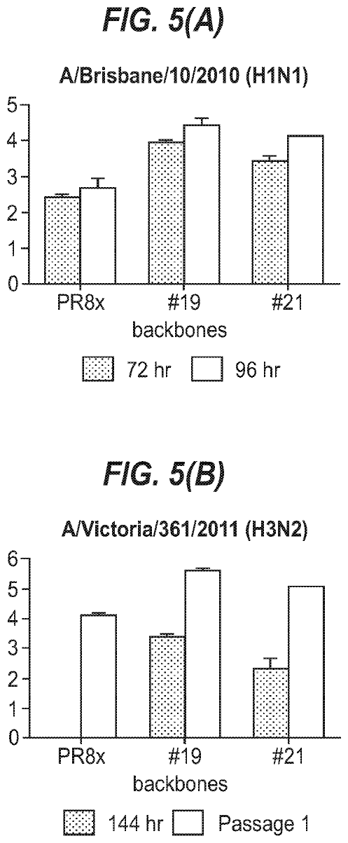

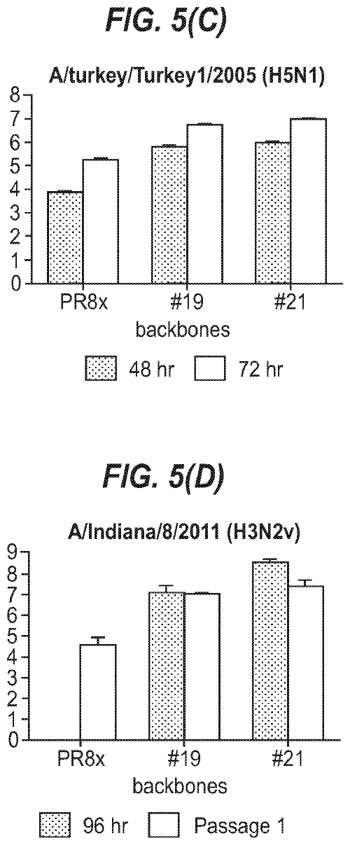

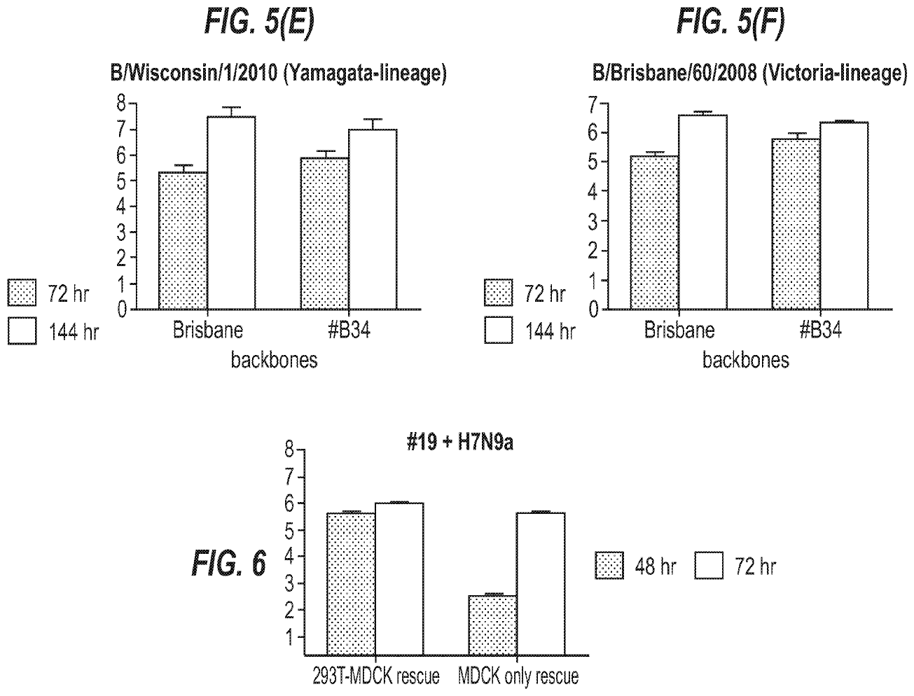

A significant increase in rescue efficiency was provided by using improved influenza backbones (sets of genome segments encoding influenza virus proteins other than HA and NA). The initial backbone improvement resulted from using genes from a PR8 variant (designated PR8x) that had been adapted over five passages to growth in MDCK 33016PF cells. Additional improvements resulted from combining backbone genome segments of multiple strains. During pilot manufacturing of influenza vaccines using MDCK 33016PF cells, several human influenza viruses, such as strain 105p30 (an A/New Caledonia/20/1999 (H1N1)-like strain that was passaged 30 times in MDCK 33016PF cells), were adapted to grow efficiently in cultured cells, although not as efficiently as strain PR8x. Synthesized viruses with HA and NA genes from historical H3N2 strains and a backbone (designated #19) composed of NP, PB1, and PB2 genome segments from strain 105p30 and M, NS, and PA genome segments from strain PR8x often outperformed equivalent viruses with entirely PR8x backbones in reverse genetic rescue efficiency and yield of HA (table 1 and FIG. 5(A), FIG. 5(B), FIG. 5(C), FIG. 5(D), FIG. 5(E), and FIG. 5(F)). Similarly, synthesized viruses with HA and NA genes from H1N1 strains and a backbone (designated #21) with the PB1 genome segment of A/California/7/2009 and the other genome segments from strain PR8x often had greater rescue efficiencies and HA yields than equivalent viruses with entirely PR8x backbones (table 1 and FIG. 5(A), FIG. 5(B), FIG. 5(C), FIG. 5(D), FIG. 5(E), and FIG. 5(F)). This finding is consistent with a report that the A/California PB1 genome segment is preferentially found in the reassortant progeny of co-infections of chicken eggs with A/California/7/2009 and a donor strain that has a PR8 backbone (18).

TABLE 1
Representative data showing virus titers and HA yields (in mass per
volume of cell culture medium before purification) from synthetic
influenza viruses relative to conventional vaccine viruses (reference
strains obtained from the US CDC or the UK National Institute for
Biological Standards and Control) in MDCK 33016PF cells.

| | | | HA | | |
|---|---|---|---|---|---|
| | | | yield | HA | |
| | | | by | yield | |
| | Reference | FFA | RP- | by | Best |
| Synthetic H1N1 strain | strain | titer | HPLC | ELISA | backbone |
| A/Christchurch/16/ | NIB74^{b} | 4.9 | 1.6 | 2.3 | #21 |
| 2010^{a,b} | | | | | |
| A/Brisbane/10/2010^{a} | wild-type | 19 | 2.1 | 7.2 | #21 |
| A/Brisbane/59/2007 | IVR-148 | 5.5 | 1.9 | 2.9 | #21 |
| A/Solomon/3/2006 | IVR-145 | 3.4 | 1.8 | 5.9 | #21 |
| Synthetic H3N2 strain | | | | | |
| A/Victoria/361/2011^{a,b} | IVR-165^{b} | 2.6 | 2.5 | 1.4 | PR8x |
| A/Victoria/210/2009^{a} | X187 | 2.6 | 2.3 | 1.7 | PR8x |
| A/Wisconsin/15/2009^{b} | X183^{b} | 35 | below | 15 | #19 |
| | | | detection | | |
| A/Uruguay/716/2007^{b} | X175C^{b} | 2.0 | 1.3 | 1.4 | #19 |
| Synthetic H5N1 strain | | | | | |
| A/turkey/Turkey/1/ | NIBRG23^{b} | 1.9 | 1.6 | n/a | #19 |
| 2005^{a,b} | | | | | |
| Synthetic H3N2v strain | | | | | |
| A/Indiana/8/2011^{a,b} | X213^{b} | 1.9 | 2.3 | n/a | #21 |
| Synthetic B-Yamagata | | | | | |
| strain | | | | | |
| B/Wisconsin/1/2010^{a,b} | wild-type^{b} | 1.7 | 1.4 | 1.7 | Brisbane |
| B/Brisbane/3/2007 | wild-type | 0.88 | 3.5 | 5.2 | #B34 |
| Synthetic B-Victoria | | | | | |
| strain | | | | | |
| B/Brisbane/60/2008^{a} | wild-type | 0.72 | 1.8 | 0.67 | Brisbane |

Data values are normalized and shown as fold-improvement over reference strains, where values of the reference strains are set to 1.0. RP-HPLC or lectin-capture ELISA was used to detect HA antigen directly from the culture medium of virus-infected MDCK cells (m.o.i = 0.001 or 0.0001), unless specified.
^{a}recombinant viruses containing synthetic HA and NA segments
^{b}viruses from culture medium were purified by sucrose-density gradient prior to characterization
n/a = data not available because strain-specific anti-sera were not available for ELISA below detection = data not available because the reference strain had undetectable HA levels by RP-HPLC

Historically, most influenza type B vaccine seeds have been wild type viruses, not reassortants, because wild type influenza B viruses generally provide adequate yields. To use the synthetic procedures for influenza B viruses more readily, two optimized type B backbones that provide consistent rescue of synthetic influenza B viruses were developed (table 1 and FIG. 5(A), FIG. 5(B), FIG. 5(C), FIG. 5(D), FIG. 5(E), and FIG. 5(F)). In the first (designated Brisbane), all backbone genome segments originate from B/Brisbane/60/2008; in the second (designated #B34), the genome segments encoding PA, PB1, PB2, and NP originate from B/Brisbane/60/2008, and those encoding M and NS originate from B/Panama/45/1990.

Overall, the use of optimized backbones for A strains increased rescue efficiencies up to 1000-fold (as measured by infectious titers obtained after transfection, FIG. 5(A), FIG. 5(B), FIG. 5(C), FIG. 5(D), FIG. 5(E), and FIG. 5(F)) and increased HA yields in research scale infections of MDCK 33016PF cells by 30% to 15-fold, depending on the strain and assay used for HA detection (table 1). In general, yields of HA from these viruses are also increased relative to those from viruses with PR8 backbones when the viruses are propagated in embryonated chicken eggs (table 2). To make use of such strain-specific differences, an optimal synthetic seed generation strategy would combine the HAs and NAs from circulating strains of interest with a panel of alternative backbones to maximize the chances of isolating a high-yielding vaccine virus.

TABLE 2
Representative data showing virus titers and HA yields (in mass per volume of egg
allantoic fluid before purification) from synthetic influenza viruses relative to conventional vaccine
viruses (reference strains obtained from the US CDC or the UK National Institute for Biological
Standards and Control) in chicken eggs.

| | | | HA titer by | HA yield | HA | |
|---|---|---|---|---|---|---|
| | Reference | | GP-RBC | by RP- | yield by | Best |
| Synthetic strain | strains | FFA titer | agglutination | HPLC | ELISA | backbone |
| A/H1N1/Christchurch/16/2010^{b} | NIB74 | 3.0 | 3.5 | 18 | 8.4 | #21 |
| A/H3N2/Victoria/210/2009^{b} | X187 | 0.94 | 1.3 | not tested | 1.2 | PR8x |
| A/H3N2/Victoria/361/2011^{b} | IVR-165 | 6.4 | 2.6 | not tested | 3.4 | #21 |
| A/H3N2v/Indiana/8/2011a,^{b} | X213 | not tested | 3.0 | 1.6 | n/a | PR8x |
| B/Yam/Wisconsin/1/2010^{a} | wild-type | 4.7 | 3.4 | not tested | 3.5 | Brisbane |
| B/Vic/Brisbane/60/2008^{a} | wild-type | 1.1 | 0.82 | not tested | 0.79 | Brisbane |

Data values are normalized and shown as fold-improvement over reference strains, where valuesof the reference strains are set to 1.0. GP-RBC agglutination, RP-HPLC or lectin-capture ELISA was used to detect HA antigen directly from the allantoic fluid of virus-infected chicken eggs, unless specified.
^{a}= recombinant viruses containing synthetic HA and NA genome segments
^{b}= viruses from egg allantoic fluid were purified by sucrose density gradient before characterization
n/a = data not available because strain-specific antisera were not available for ELISA
not tested = data not available because assay was not performed

Speed of Synthetic Vaccine Virus Generation in a Simulated Pandemic Response.

In a timed proof-of-concept test of the synthetic system's first iteration, the virus synthesis group was provided with unidentified HA and NA genome segment sequences by collaborators not directly involved in the synthesis (17). The sequences included complete coding regions but incomplete un-translated regions (UTRs), mimicking the information likely to be available in the early days of a pandemic. Sequence analysis of the HA genome segment showed that it was very closely related (96% nucleotide sequence identity by Blast to GenBank) to a low pathogenicity North American avian H7N3 virus (A/Canada goose/BC/3752/2007), and that the NA genome segment was very closely related (96% nucleotide sequence identity by Blast to GenBank) to a low pathogenicity North American avian H1ON9 virus (A/king eider/Alaska/44397-858/2008). Although our software generates the sequences of the oligonucleotides used for rescue, user intervention is needed when there are ambiguities in the available sequence data. In this case, the unknown terminal UTR sequences were generated based on sequence alignments with a limited number of related full-length H7 sequences and by comparison with consensus UTRs for H7 and N9 genomic segments created from high quality sequence data in GenBank. This analysis revealed heterogeneity in the non-coding regions of NA genes of H7N9 strains (U/C at 1434 in the positive-sense orientation). So, alternative sets of 5′ NA oligonucleotides were used to construct two variants of the NA cassettes.

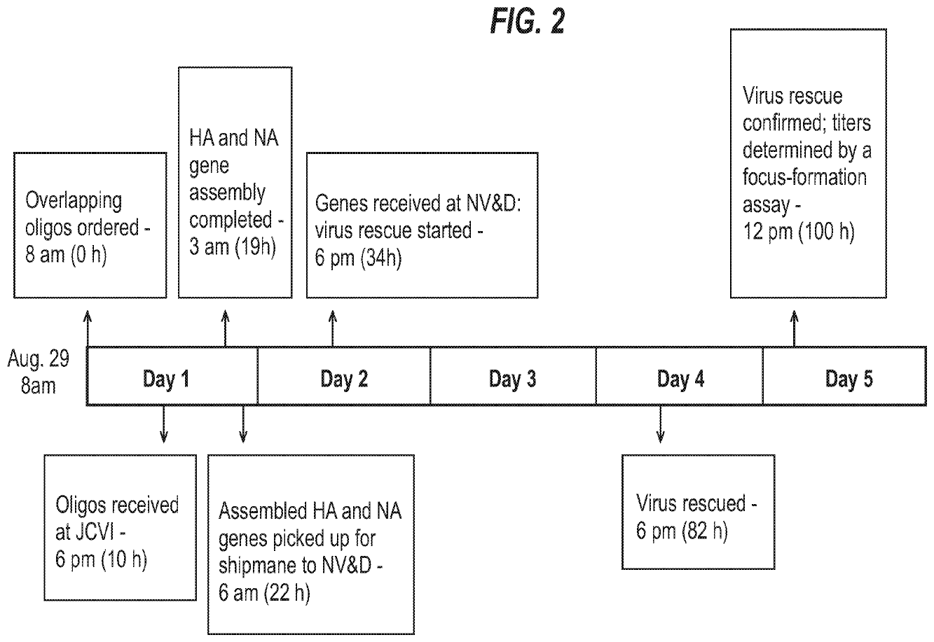

Oligonucleotide synthesis began at 8:00 am EDT on Monday, Aug. 29, 2011 (FIG. 2). By noon on Friday, September 4, immunostaining of a secondary culture confirmed that the virus had been rescued. The 4 days and 4 hours from start of synthesis to detection of rescued virus included time spent shipping DNA from the oligonucleotide synthesis and gene assembly laboratories in California to the virus rescue laboratory in Massachusetts. When all functions are consolidated in one location, the potential for delays and mishaps due to shipping will be reduced. The original proof-of-concept rescues were conducted using 293T cells; rescue of the strains using MDCK cells, as would be done during an actual pandemic response, slows detection of rescued virus by approximately 24 hours (FIG. 6). The sequences of the HA and NA genome segments of the synthetic H7N9 reassortant viruses from the proof-of-concept exercise were determined following two rounds of virus amplification in MDCK 33016PF cells and were identical to those used to program oligonucleotide synthesis. Two-way hemagglutination inhibition (HI) testing (reciprocal HI assays using antigen from the synthetic and natural strains and ferret sera drawn after synthetic and natural virus infection) (19, 20) demonstrated antigenic identity of the synthetic virus to A/goose/Nebraska/17097-4/2011 (H7N9), which had subsequently been revealed as the wild type virus from which the sequences that were electronically transmitted to the virus synthesis group had been obtained (Table 1).

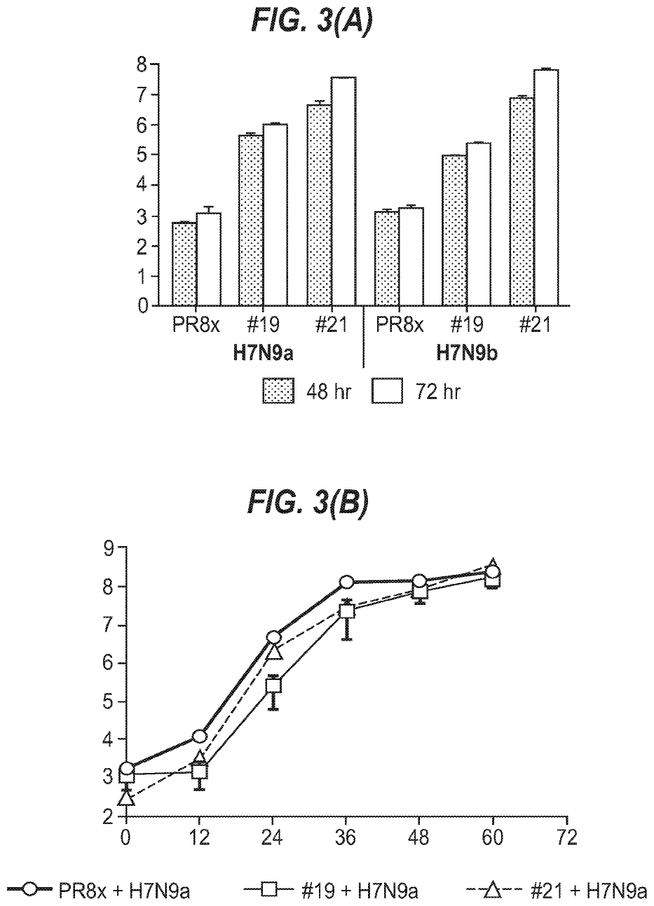

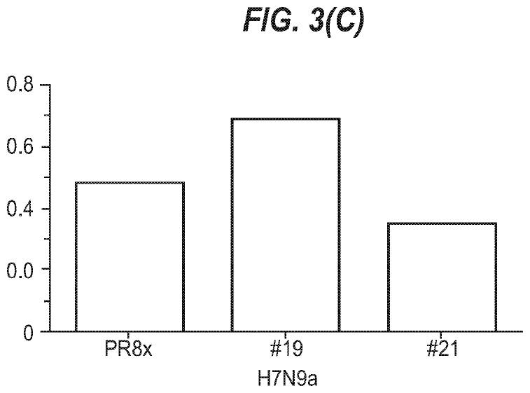

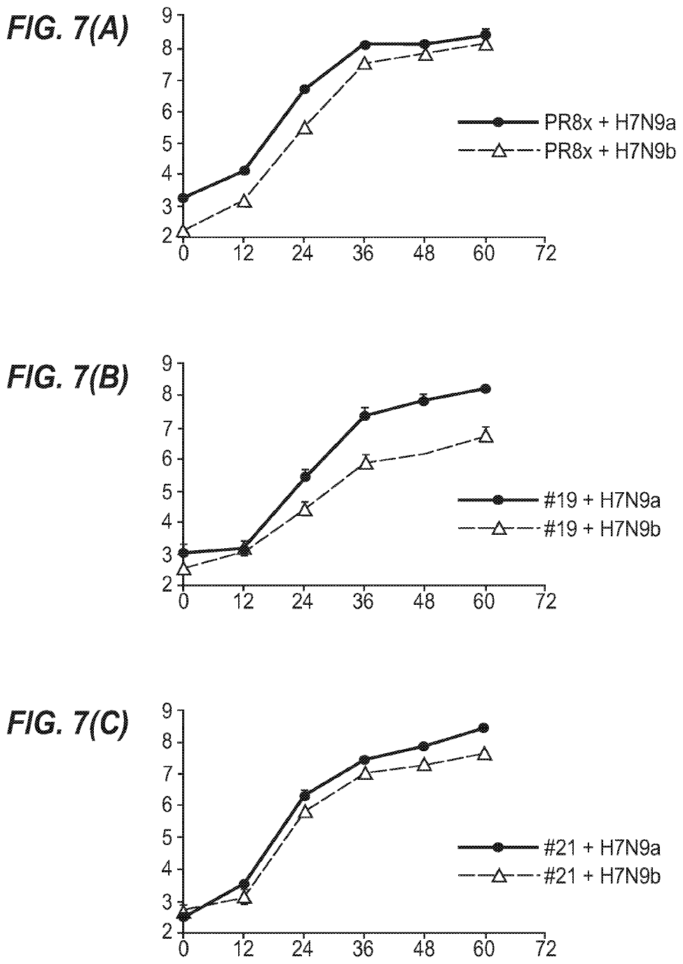

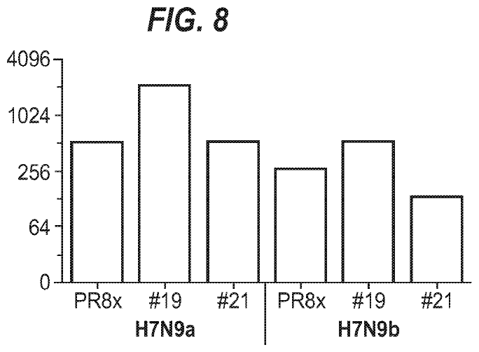

The A/goose/Nebraska/17097-4/2011 HA and NA genes were rescued with PR8x, #19, and #21 backbones. Virus rescue was more efficient using the #19 and #21 backbones than the PR8x backbone, based on the titers of viruses harvested 48 and 72 hours after transfection (FIG. 3(A)). To test growth characteristics, the synthetic viruses were amplified once in MDCK 33016 PF monolayers and then used to infect suspension MDCK 33016PF cultures at a multiplicity-of-infection (m.o.i.) of 0.001. Despite differences in the efficiency of virus recovery, viruses exhibited similar growth characteristics, regardless of backbone (FIG. 3(B)). The H7N9a set of viruses (C1434 positive sense NA) achieved infectious titers approximately 10-fold higher than their H7N9b counterparts (U1434 positive sense NA; FIG. 7(A), FIG. 7(B), and FIG. 7(C)). The viruses with the highest infectious yields also produced the most HA per volume of infected MDCK suspension culture (FIG. 3(C)). Thus, the single nucleotide substitution in the 5′ NA non-coding region of the genomic RNA strongly influenced both infectious titer and HA yield (FIG. 8). The H7N9a virus with the #19 backbone produced 1.5-fold more HA than a virus with the same HA and NA in the context of the standard PR8x backbone (FIG. 3(C)). This demonstration confirmed the importance of rescuing multiple HA or NA variants with multiple backbones to increase the probability of identifying high yielding vaccine virus strains early in the vaccine seed generation process. Simultaneous rescue of multiple variants is faster and more easily accomplished using the synthetic approach than standard plasmid mutagenesis approaches. This example also indicates the importance for pandemic response of including as complete genome segment sequences as possible in genetic databases and of clearly delineating terminal sequences originating from viral genome segments from those originating from sequencing primers.

Robustness of the Synthetic Approach to Vaccine Virus Generation.

By combining gene synthesis, enzymatic error correction, optimized rescue protocols, and optimized backbones, the synthetic approach provides a robust tool to obtain influenza vaccine viruses. To date, the team has not encountered any influenza virus strain that cannot be rescued synthetically. The synthetic process has been used to generate a wide variety of influenza strains, including H1N1 (pre- and post-2009 variants), seasonal H3N2, swine origin H3N2v, B (Yamagata and Victoria lineages), attenuated H5N1, and H7N9 strains (table 3). The robustness of synthetic influenza virus recovery on MDCK cells is in striking contrast to the unreliability of conventional vaccine virus isolation using eggs, particularly for recent H3N2 strains (21).

TABLE 3
Diversity of synthetic influenza virus strains rescued.

| SEASONAL SEROTYPE A VIRUSES | Backbone | | |
|---|---|---|---|
| Source of synthetic HA NA | PR8X | #19 | #21 |
| A/H1N1/Brisbane/10/2010 | + | + | + |
| A/H1N1/Christchurch/16/2010 (NIB74) | + | + | + |
| A/H1N1/Christchurch/16/2010 NIB74-K170E | n/a | n/a | + |
| A/H1N1/Christchurch/16/2010 NIB74-K171E | n/a | n/a | + |
| A/H1N1/Christchurch/16/2010 NIB74-G172E | n/a | n/a | + |
| A/H1N1/Christchurch/16/2010 NIB74-G173D | n/a | n/a | + |
| A/H3N2/Uruguay/716/2007 | + | + | + |
| A/H3N2/Victoria/210/2009 (X187) | + | + | + |
| A/H3N2/Victoria/361/2011 (CDC E3) | + | + | + |
| A/H3N2/Victoria/361/2011 (WHO E3) | + | + | + |
| A/H3N2/Victoria/361/2011 (MDCK) | + | + | + |
| A/H3N2/Berlin/93/2011 (egg-derived) | + | + | + |
| A/H3N2/Berlin/93/2011 (cell-derived) | + | + | + |
| A/H3N2/Brisbane/402/2011 | + | + | + |
| A/H3N2/Victoria/304/2011 NVD p2/E3 | − | − | + |
| A/H3N2/Brisbane/256/2011 MDCK P2 | + | + | + |
| A/H3N2/Brisbane/256/2011 P2/E3 | − | + | + |
| A/H3N2/South Australia/34/2011 | − | + | + |
| A/H3N2/Brisbane/299/2011 (IVR164) | + | + | + |
| A/H3N2/Brisbane/299/2011 (E5) | + | + | + |
| A/H3N2/South Australia/3/2011 | + | + | + |
| A/H3N2/Wisconsin/1/2001 | + | + | + |

| SEASONAL SEROTYPE B VIRUSES | Backbone | |
|---|---|---|
| Source of synthetic HA NA | Bris | #B34 |
| B/Yam/Hubei-Wujiangang/158/2009 | + | + |
| B/Yam/Wisconsin/1/2010 | + | + |
| B/Yam/Brisbane/3/2007 | + | + |
| B/Yam/Jiangsu/10/2003 | + | + |
| B/Yam/Johannesburg/05/1999 | + | + |
| B/Yam/Yamanashi/166/1998 | + | + |
| B/Yam/Yamagata/16/1988 | + | + |
| B/Yam/Texas/6/2011 | + | − |
| B/Vic/New Hampshire/1/2012 | + | + |
| B/Vic/Malaysia/2506/2004 | + | + |
| B/Vic/Brisbane/32/2002 | + | + |
| B/Vic/Brisbane/60/2008 (cell) | + | + |
| B/Vic/Brisbane/60/2008 (egg) | + | n/a |
| B/Vic/Nevada/3/2011 | + | + |

| PANDEMIC VIRUSES | Backbone | | |
|---|---|---|---|
| Source of synthetic HA NA | PR8X | #19 | #21 |
| A/H5N1/Hubei/1/2010 | + | + | + |
| A/H5N1/Egypt/N03072/2010 | + | + | + |
| A/H5N1/Turkey/Turkey/1/2005 | + | + | + |
| A/H7N9/goose/Nebraska/11-017097-4/2011 | + | + | + |
| A/H3N2v/Indiana/8/2011 | + | + | + |

n/a = not attempted;
+ = virus recovered in ≤6 days post-transfection;
− = virus not recovered by 6 days post-transfection.

Implications for the Global Strain Change and Pandemic Response Systems.

The speed, ease, and accuracy with which higher yielding influenza vaccine seeds can be produced using synthetic techniques promises more rapid future pandemic responses and a more reliable supply of better matched seasonal and pandemic influenza vaccines. The potential for propagation of adventitious agents from the human nasal secretions used for original influenza virus isolation will be eliminated when such materials are used only to generate sequence information, not for propagation into viruses used to seed vaccine production bioreactors or eggs. The speed of the technical steps of synthesis and virus rescue is actually a relatively minor component of the potential acceleration of seed generation based on synthetic technology. If the performance of synthetic vaccine viruses is sufficient, much greater time savings will result from the ability of synthetic technology to alleviate the need to ship viruses and clinical specimens between laboratories and use a classic reassortment approach to generate high-yielding vaccine strains.

Today, the more than 120 National Influenza Centers (NICs) that conduct influenza surveillance periodically ship clinical specimens to WHO Collaborating Centers, where attempts are made to propagate the wild type viruses in MDCK cells. With synthetic vaccine viruses, the system could realize increased efficiency. Sequence data obtained by directly sequencing HA and NA genomic RNAs in clinical specimens at the NICs could be posted on publically accessible websites, where they can be downloaded immediately by manufacturers, public health agencies, and other researchers worldwide. Continuous comparison of the stream of sequence data to databases of sequence and HI data by algorithms now under development could identify those emerging viruses that are most likely to have significant antigenic differences from current vaccine strains. Efficient primary synthetic rescue with a panel of high growth backbones will simultaneously generate the viruses needed for antigenic testing and the best vaccine seed candidates to be used if a virus is found to be antigenically distinct and epidemiologically important.

Today, vaccine viruses are only shipped from WHO Collaborating Centers or reassortant generating laboratories to manufacturers after they are fully tested, and testing often takes longer than the generation of the vaccine strains. The decentralization of seed generation permitted by these synthetic techniques could allow manufacturers to undertake scale up and process development at risk for strains that they could generate immediately after the NICs post sequences. Carrying out these manufacturing activities simultaneously with seed testing would cut additional weeks from pandemic response times. Libraries of synthetic influenza genes could further accelerate pandemic responses, if the pre-synthesized genes in the libraries match future pandemic strains.

Growth Characteristics of Reassortant Viruses Containing PR8-X or Canine Adapted PR8-X Backbones

In order to provide high-growth donor strains, the inventors found that a reassortant influenza virus comprising the PB1 segment of A/California/07/09 and all other backbone segments from PR8-X shows improved growth characteristics compared with reassortant influenza viruses which contain all backbone segments from PR8-X. This influenza backbone is referred to as #21.

In order to test the suitability of the #21 strain as a donor strain for virus reassortment, reassortant influenza viruses are produced by reverse genetics which contain the HA and NA proteins from various influenza strains (including zoonotic, seasonal, and pandemic-like strains) and the other viral segments from either PR8-X or the #21 backbone. The HA content, HA yield and the viral titres of these reassortant viruses are determined. As a control a reference vaccine strain which does not contain any backbone segments from PR8-X or A/California/07/09 is used. These viruses are cultured either in embyronated chicken eggs or in MDCK cells.

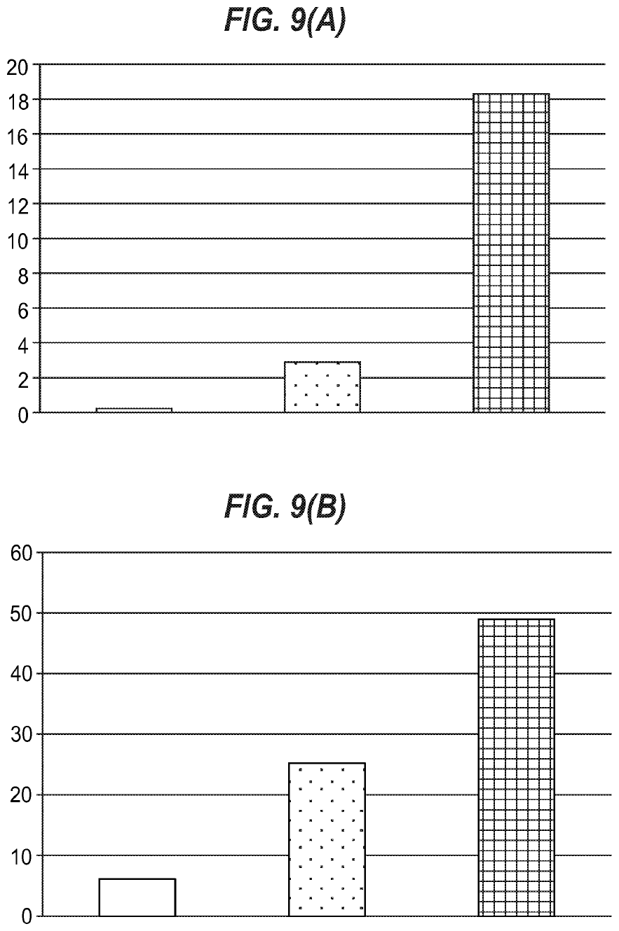

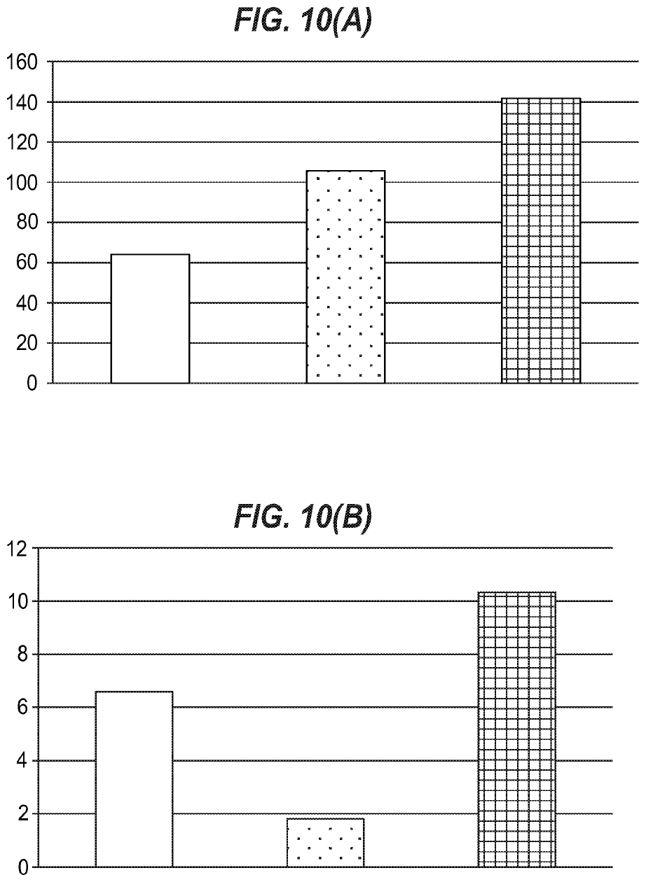

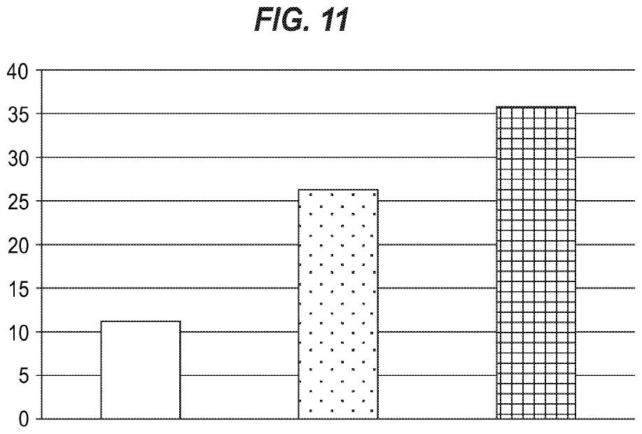

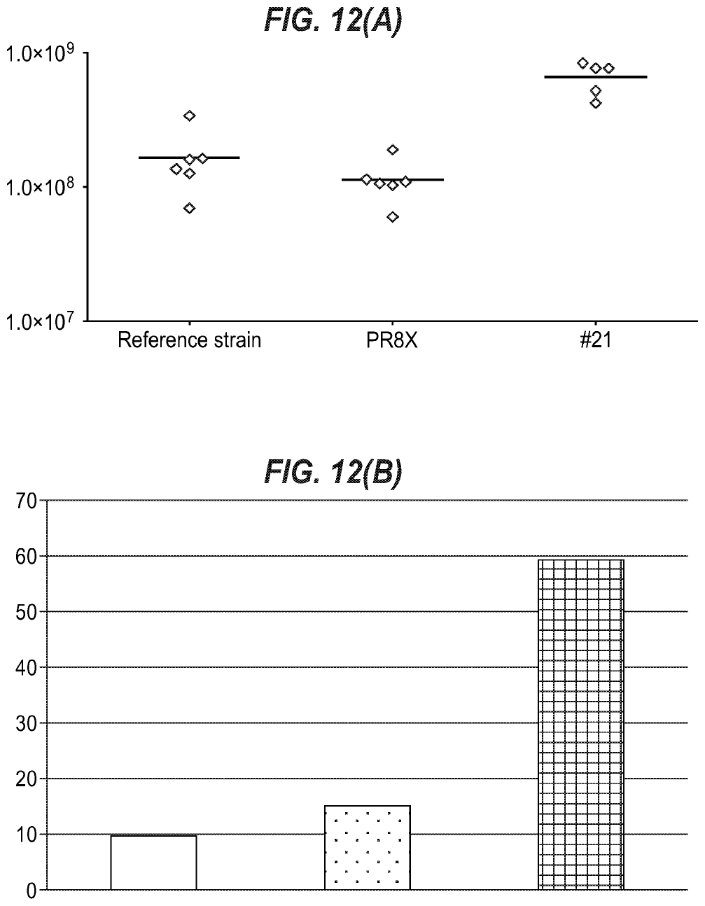

The results indicate that reassortant viruses which contain the #21 backbone consistently give higher viral titres and HA yields compared with the control virus and the virus which contains all backbone segments from PR8-X in both eggs and cell culture. This difference is due to the PB1 segment because this is the only difference between #21 reassortants and PR8-X reassortants (see FIG. 8, FIG. 9(A), FIG. 9(B), FIG. 10(A), FIG. 10(B), and FIG. 11).

In order to test the effect of canine-adapted mutations on the growth characteristics of PR8-X, the inventors introduce mutations into the PA segment (E327K, N444D, and N675D), or the NP segment (A27T, E375N) of PR8-X. These backbones are referred to as PR8-X(cPA) and PR8-X(cNP), respectively. Reassortant influenza viruses are produced containing the PR8-X(cPA) and PR8-X(cNP) backbones and the HA and NA segments of a pandemic-like H1 influenza strain (strain 1) or a H3 influenza strain (strain 2). As a control a reference vaccine strain which does not contain any backbone segments from PR8-X is used. The reassortant influenza viruses are cultured in MDCK cells.

The results show that reassortant influenza viruses which contain canine-adapted backbone segments consistently grow to higher viral titres compared with reassortant influenza viruses which contain unmodified PR8-X backbone segments (see FIG. 8, FIG. 9(A), and FIG. 9(B)).

Growth Characteristics of Reassortant Viruses Containing PR8-X, #21 or #21C Backbones

In order to test whether canine-adapted mutations in the backbone segments improve the growth characteristics of the #21 backbone, the inventors modify the #21 backbone by introducing mutations into the PR8-X PB2 segment (R389K, T559N). This backbone is referred to as #21C. Reassortant influenza viruses are produced by reverse genetics which contain the HA and NA proteins from two different pandemic-like H1 strains (strains 1 and 2) and the other viral segments from either PR8-X, the #21 backbone or the #21C backbone. As a control a reference vaccine strain which does not contain any backbone segments from PR8-X or A/California/07/09 is used. These viruses are cultured in MDCK cells. The virus yield of these reassortant viruses is determined. For reassortant influenza viruses containing the HA and NA segments from the pandemic-like H1 strain (strain 1) and the PR8-X or #21C backbones the HA titres are also determined.

The results show that reassortant influenza viruses which contain the #21C backbone consistently grow to higher viral titres compared with reassortant influenza viruses which contain only PR8-X backbone segments or the #21 backbone (see FIG. 5(A), FIG. 5(B), FIG. 5(C), FIG. 5(D), FIG. 5(E), and FIG. 5(F), FIG. 6, FIG. 7(A), FIG. 7(B), and FIG. 7(C)). Reassortant influenza viruses comprising the #21C backbone also show higher HA titres compared with PR8-X reassortants.

Growth Characteristics of Reassortant Influenza B Viruses

Reassortant influenza B viruses are produced by reverse genetics which contain the HA and NA proteins from various influenza strains and the other viral segments from B/Brisbane/60/08 and/or B/Panama/45/90. As a control the corresponding wild-type influenza B strain is used. These viruses are cultured either in embyronated chicken eggs or in MDCK cells. The following influenza B strains are used:

TABLE 4

| | Backbone segments | | | | | | Antigenic determinants | |
|---|---|---|---|---|---|---|---|---|
| combo # | PA | PB1 | PB2 | NP | NS | M | HA | NA |
| 1 (WT) | Brisbane | Brisbane | Brisbane | Brisbane | Brisbane | Brisbane | Brisbane | Brisbane |
| 2 | Panama | Brisbane | Brisbane | Brisbane | Brisbane | Brisbane | Brisbane | Brisbane |
| 3 | Brisbane | Panama | Brisbane | Brisbane | Brisbane | Brisbane | Brisbane | Brisbane |
| 4 | Brisbane | Brisbane | Panama | Brisbane | Brisbane | Brisbane | Brisbane | Brisbane |
| 5 | Brisbane | Brisbane | Brisbane | Panama | Brisbane | Brisbane | Brisbane | Brisbane |
| 6 | Panama | Panama | Brisbane | Brisbane | Brisbane | Brisbane | Brisbane | Brisbane |
| 7 | Panama | Brisbane | Panama | Brisbane | Brisbane | Brisbane | Brisbane | Brisbane |
| 8 | Panama | Brisbane | Brisbane | Panama | Brisbane | Brisbane | Brisbane | Brisbane |
| 9 | Brisbane | Panama | Panama | Brisbane | Brisbane | Brisbane | Brisbane | Brisbane |
| 10 | Brisbane | Panama | Brisbane | Panama | Brisbane | Brisbane | Brisbane | Brisbane |
| 11 | Brisbane | Brisbane | Panama | Panama | Brisbane | Brisbane | Brisbane | Brisbane |
| 12 | Panama | Panama | Panama | Brisbane | Brisbane | Brisbane | Brisbane | Brisbane |
| 13 | Panama | Panama | Brisbane | Panama | Brisbane | Brisbane | Brisbane | Brisbane |
| 14 | Panama | Brisbane | Panama | Panama | Brisbane | Brisbane | Brisbane | Brisbane |
| 15 | Brisbane | Panama | Panama | Panama | Brisbane | Brisbane | Brisbane | Brisbane |
| 16 | Panama | Panama | Panama | Panama | Brisbane | Brisbane | Brisbane | Brisbane |
| 17 | Panama | Panama | Panama | Panama | Panama | Panama | Brisbane | Brisbane |
| 20 | Brisbane | Panama | Panama | Panama | Panama | Panama | Panama | Panama |
| 21 | Panama | Brisbane | Panama | Panama | Panama | Panama | Panama | Panama |
| 22 | Panama | Panama | Brisbane | Panama | Panama | Panama | Panama | Panama |
| 23 | Panama | Panama | Panama | Brisbane | Panama | Panama | Panama | Panama |
| 24 | Brisbane | Brisbane | Panama | Panama | Panama | Panama | Panama | Panama |
| 25 | Brisbane | Panama | Brisbane | Panama | Panama | Panama | Panama | Panama |
| 26 | Brisbane | Panama | Panama | Brisbane | Panama | Panama | Panama | Panama |
| 27 | Panama | Brisbane | Brisbane | Panama | Panama | Panama | Panama | Panama |
| 28 | Panama | Brisbane | Panama | Brisbane | Panama | Panama | Panama | Panama |
| 29 | Panama | Panama | Brisbane | Brisbane | Panama | Panama | Panama | Panama |
| 30 | Brisbane | Brisbane | Brisbane | Panama | Panama | Panama | Panama | Panama |
| 31 | Brisbane | Brisbane | Panama | Brisbane | Panama | Panama | Panama | Panama |
| 32 | Brisbane | Panama | Brisbane | Brisbane | Panama | Panama | Panama | Panama |
| 33 | Panama | Brisbane | Brisbane | Brisbane | Panama | Panama | Panama | Panama |
| 34 | Brisbane | Brisbane | Brisbane | Brisbane | Panama | Panama | Panama | Panama |
| 35 | Brisbane | Brisbane | Brisbane | Brisbane | Brisbane | Brisbane | Panama | Panama |

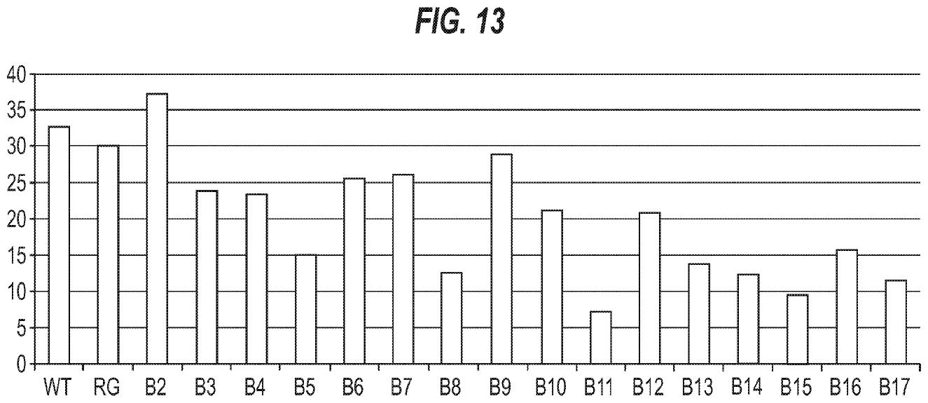

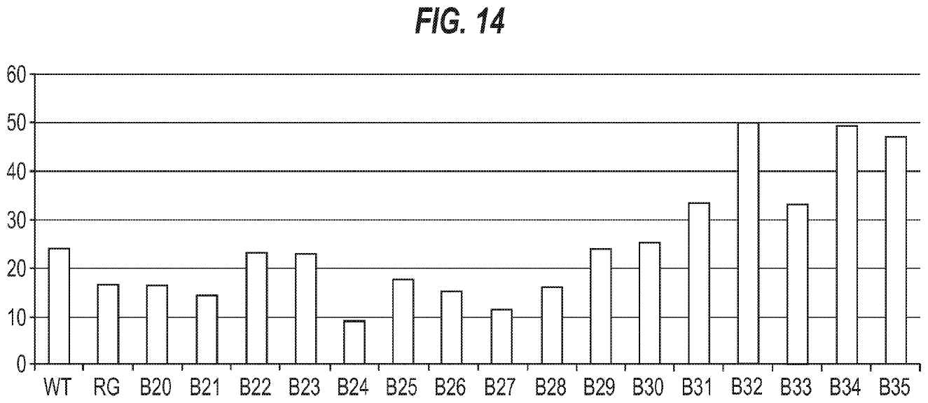

The results indicate that reassortant viruses #2, #9, #30, #31, #32, #33, #34 and #35 grow equally well or even better in the culture host (see FIGS. 13 and 14) than the corresponding wild-type strain. Most of these strains comprise the NP segment from B/Brisbane/60/08 and some (in particular those which grew best) further contain the PB2 segment from B/Brisbane/60/08. All of these viruses also contain viral segments from the BNictoria/2/87-like strain and the B/Yamagata/16/88-like strain at a ratio 7:1, 6:2, 4:4, 3:4 or 1:7.

It will be understood that the invention has been described by way of example only and modifications may be made whilst remaining within the scope and spirit of the invention

### REFERENCES

[1] WO2007/002008

[2] WO2007/124327

[3] WO2011/012999

[4] Verity et al. (2012); Influenza Other Respi Viruses;6(2):101-9.

[5] Zhou et al. (2009) J Virol.;83(19):10309-13

[6] Gibson et al. (2010); Nature Methods 7, 901-903.

[7] Gibson et al. (2009) Nature Methods 6, 343-345.

[8] U.S. Pat. No. 6,576,453

[9] Yount et al. (2002) J Virol 76:11065-78.

[10] Kodumal et al. (2004) Proc Natl Acad Sci USA. 101(44):15573-8.

[11] Yount et al. (2002) J Virol 76:11065-78.

[12] Sambrook et al, Molecular Cloning: A Laboratory Manual, 2 ed., 1989, Cold Spring Harbor Press, Cold Spring Harbor, N.Y.

[13] WO2010/133964

[14] WO2009/000891

[15] Sambrook et al, Molecular Cloning: A Laboratory Manual, 2 ed., 1989, Cold Spring Harbor Press, Cold Spring Harbor, N.Y.

[16] WO2011048560

[17] Neumann et al. (2005) Proc Natl Acad Sci USA 102: 16825-9

[18] Kistner et al. (1998) Vaccine 16:960-8.

[19] Kistner et al. (1999) Dev Biol Stand 98:101-110.

[20] Bruhl et al. (2000) Vaccine 19:1149-58.

[21] WO2006/108846.

[22] Pau et al. (2001) Vaccine 19:2716-21.

[23] http://www.atcc.org/

[24] http://locus.umdnj.edu/

[26] Brands et al. (1999) Dev Biol Stand 98:93-100.

[27] Halperin et al. (2002) Vaccine 20:1240-7.

[28] EP-A-1260581 (WO01/64846).

[29] WO2006/071563.

[30] WO2005/113758.

[31] Grachev et al. (1998) Biologicals;26(3):175-93.

[32] Herlocher et al. (2004) J Infect Dis 190(9):1627-30.

[33] Le et al. (2005) Nature 437(7062):1108.

[34] Needleman & Wunsch (1970) J. Mol. Biol. 48, 443-453.

[35] Rice et al. (2000) Trends Genet 16:276-277.

[36] Rota et al. (1992) J Gen Virol 73:2737-42.

[37] GenBank sequence GI:325176.

[38] McCullers et al. (1999) J Virol 73 :7343-8.

[39] GenBank sequence GI:325237.

[40] U.S. Pat. No. 6,468,544.

[41] WO97/37001

[42] WO02/28422.

[43] WO02/067983.

[44] WO02/074336.

[45] WO01/21151.

[46] WO02/097072.

[47] WO2005/113756.

[48] Huckriede et al. (2003) Methods Enzymol 373:74-91.

[49] Vaccines. (eds. Plotkins & Orenstein). 4th edition, 2004, ISBN: 0-7216-9688-0

[50] Treanor et al. (1996) J Infect Dis 173:1467-70.

[51] Keitel et al. (1996) Clin Diagn Lab Immunol 3:507-10.

[52] Herlocher et al. (2004) J Infect Dis 190(9):1627-30.

[53] Le et al. (2005) Nature 437(7062):1108.

[54] WO2008/068631.

[55] Gennaro (2000) Remington: The Science and Practice of Pharmacy. 20th edition, ISBN: 0683306472.

[56] Banzhoff (2000) Immunology Letters 71:91-96.

[57] Nony et al. (2001) Vaccine 27:3645-51.

[58] EP-B-0870508.

[59] US 5948410.

[60] WO2007/052163.

[61] WO2007/052061

[62] WO90/14837.

[63] Podda & Del Giudice (2003) Expert Rev Vaccines 2:197-203.

[64] Podda (2001) Vaccine 19: 2673-2680.

[65] Vaccine Design: The Subunit and Adjuvant Approach (eds. Powell & Newman) Plenum Press 1995 (ISBN 0-306-44867-X).

[66] Vaccine Adjuvants: Preparation Methods and Research Protocols (Volume 42 of Methods in Molecular Medicine series). ISBN: 1-59259-083-7. Ed. O'Hagan.

[67] WO2008/043774.

[68] Allison & Byars (1992) Res Immunol 143:519-25.

[69] Hariharan et al. (1995) Cancer Res 55:3486-9.

[70] US-2007/014805.

[71] US-2007/0191314.

[72] Suli et al. (2004) Vaccine 22(25-26):3464-9.

[73] WO95/11700.

[74] U.S. Pat. No. 6,080,725.

[75] WO2005/097181.

[76] WO2006/113373.

[77] Potter & Oxford (1979) Br Med Bull 35: 69-75.

[78] Greenbaum et al. (2004) Vaccine 22:2566-77.

[79] Zurbriggen et al. (2003) Expert Rev Vaccines 2:295-304.

[80] Piascik (2003) J Am Pharm Assoc (Wash D.C.). 43:728-30.

[81] Mann et al. (2004) Vaccine 22:2425-9.

[82] Halperin et al. (1979) Am J Public Health 69:1247-50.

[83] Herbert et a/. (1979) J Infect Dis 140:234-8.

[84] Chen et al. (2003) Vaccine 21:2830-6.

[85] Current Protocols in Molecular Biology (F.M. Ausubel et al., eds., 1987) Supplement 30.

[86] Smith & Waterman (1981) Adv. Appl. Math. 2: 482-489.

[87] Suphaphiphat et al. (2010), Virology J. 7, 157.

[88] Hoffmann et al. (2000) PNAS 97, 6108-6113.

[89] Nicolson et al. (2005) Vaccine 23, 2943-2952.

[90] Ozaki et al (2004) J. Virol. 78, 1851-1857.

[91] Boettcher et al. (2006) J. Virol. 80, 9896-9898.

1. A method of preparing an influenza virus, comprising:
a) preparing one or more expression construct(s) which comprise(s) coding sequences for expressing at least one segment of an influenza virus genome;
b) introducing into a cell which is not 293T one or more expression construct(s) which encode(s) the viral segments of an influenza virus, wherein at least one expression construct is the expression construct prepared in step (a); and
c) culturing the cell in order to produce a reassortant influenza virus from the express construct(s) introduced in step (b);
wherein steps (a) to (c) are performed in a time period of 124 hours or less.

2. A method of preparing an influenza virus comprising the steps of
a) preparing one or more expression construct(s) which comprise(s) coding sequences for expressing at least one segment of an influenza virus genome;
b) introducing into a cell one or more expression construct(s) which encode(s) the viral segments of an influenza virus, wherein at least one expression construct is the expression construct prepared in step (a); and
c) culturing the cell in order to produce a reassortant influenza virus from the expression construct(s) introduced in step (b);
wherein steps (a) to (c) are performed in a time period of 100 hours or less.

3. The method of claim 1, wherein the cell is a non-human cell or a human non-kidney cell.

4. A method of preparing a reassortant influenza virus, comprising:
a) providing a synthetic expression construct which comprises coding sequences for expressing at least one segment of an influenza virus genome by (i) synthesising a plurality of overlapping fragments of the synthetic expression construct, wherein the overlapping fragments span the complete synthetic expression construct, and (ii) joining the fragments to provide the synthetic expression construct;
b) introducing into a cell which is not 293T one or more expression construct(s) which encode(s) the viral segments required to produce an influenza virus, wherein at least one expression construct is the synthetic expression construct prepared in step (a); and
c) culturing the cell in order to produce a reassortant influenza virus from the viral segments introduced in step (b);
wherein steps (a) to (c) are performed in a time period of 124 hours or less.

5. The method of claim 4, wherein the cell is a non-human cell or a human non-kidney cell.

6. The method of claim 1, further comprising (d) contacting a cell which is of the same cell type as the cell used in step (c) with the virus produced in step (c) to produce further reassortant influenza virus.

7. A method of preparing an influenza virus, comprising:
a) providing a synthetic expression construct which comprises coding sequences for expressing at least one segment of an influenza virus genome by (i) synthesising a plurality of overlapping fragments of the synthetic expression construct, wherein the overlapping fragments span the complete synthetic expression construct, and (ii) joining the fragments to provide the synthetic expression construct;
b) introducing into a cell one or more expression construct(s) which encode(s) the viral segments of an influenza virus, wherein at least one expression construct is the synthetic expression construct prepared in step (a);
c) culturing the cell in order to produce a reassortant influenza virus from the viral segments introduced in step (b); and
d) contacting a cell which is of the same cell type as the cell used in step (c) with the virus produced in step (c) to produce further reassortant influenza virus;
wherein steps (a) to (c) are performed in a time period of 124 hours or less.

8. The method of claim 7, wherein the cell used in steps (c) and (d) is not 293T.

9. The method of claim 7, wherein the cell used in steps (c) and (d) is a non-human cell or a human non-kidney cell.

10. The method of claim 1, wherein the synthetic expression construct comprises coding sequences for the HA and/or NA segment.

11. The method of claim 1, wherein the synthetic expression construct is linear.

12. The method of claim 1, wherein the fragments have a length between 61 and 100 nucleotides.

13. The method of claim 12, wherein the fragments have a length between 61 and 74 nucleotides.

14. The method of claim 1, wherein the fragments have an overlap of about 40 nucleotides.

15. The method of claim 1, wherein at least part of the synthetic expression construct obtained in step (a) is amplified.

16. The method of claim 1, wherein the step of providing the synthetic expression construct comprises: (i) synthesising a plurality of overlapping fragments of the synthetic expression construct, wherein the overlapping fragments span the complete synthetic expression construct; (ii) joining the fragments to provide a DNA molecule; (iii) melting the DNA molecule; (iv) re-annealing the DNA in the presence of an agent which excises mismatched nucleotides from the DNA molecule; and (v) amplifying the DNA to produce the synthetic expression construct.

17. The method of claim 1, wherein the reassortant influenza virus is a reassortant influenza A virus.

18. The method of claim 17, wherein the reassortant influenza A virus comprises one or more backbone segments having at least 95%, at least 96%, at least 97%, at least 98%, at least 99% or 100% identity to the sequence of SEQ ID NOs 9 to 14.

19. The method of claim 17, wherein the reassortant influenza A virus comprises one or more backbone segments having at least 95%, at least 96%, at least 97%, at least 98%, at least 99% or 100% identity to the sequence of SEQ ID NOs 42 to 47.

20. The method of claim 17, wherein the reassortant influenza A virus comprises backbone segments from two or more influenza A strains.

21. The method of claim 17, wherein the reassortant influenza A virus comprises the PB1 segment of SEQ ID NO:
43; the PB2 segment of SEQ ID NO: 44; the PA segment of SEQ ID NO: 9; the NP segment of SEQ ID NO: 45; the M segment of SEQ ID NO: 13; and the NS segment of SEQ ID NO: 14.

22. The method of claim 17, wherein the reassortant influenza A virus comprises the PB1 segment of SEQ ID NO: 18; the PB2 segment of SEQ ID NO: 11; the PA segment of SEQ ID NO: 9; the NP segment of SEQ ID NO: 12; the M segment of SEQ ID NO: 13; and the NS segment of SEQ ID NO: 14.

23. The method of claim 1, wherein the reassortant influenza virus is a reassortant influenza B virus.

24. The method of claim 23, wherein the reassortant influenza B viruses comprises the PA segment 25 of SEQ ID NO: 71, the PB1 segment of SEQ ID NO: 72, the PB2 segment of SEQ ID NO: 73, the NP segment of SEQ ID NO: 74, the NS segment of SEQ ID NO: 76 and the M segment of SEQ ID NO: 75.

25. The method of claim 23, wherein the reassortant influenza B viruses comprises the PA segment of SEQ ID NO: 71, the PB 1 segment of SEQ ID NO:
72, the PB2 segment of SEQ ID NO: 73, the NP segment of SEQ ID NO: 74, the NS segment of SEQ ID NO: 76 and the M segment of SEQ ID NO: 81.

26. A method of preparing an influenza vaccine, comprising:
a) contacting a cell with a reassortant influenza virus prepared by the method of claim 1;
b) culturing the cell in order to produce an influenza virus; and
c) preparing a vaccine from the influenza virus produced in step (b).

27. The method of claim 26, wherein the cell is a human non-kidney cell or a non-human cell.

28. The method of claim 26, wherein the cell used in step (a) is of the same cell type as the cell used to prepare the reassortant influenza virus.

29. The method of claim 26, wherein step (c) involves inactivating the virus.

30. The method of claim 26, wherein the vaccine is a whole virion vaccine.

31. The method of claim 26, wherein the vaccine is a split virion vaccine.

32. The method of claim 26, wherein the vaccine is a surface antigen vaccine.

33. The method of claim 26, wherein the vaccine is a virosomal vaccine.

34. The method of claim 26, wherein the vaccine contains less than 10 ng of residual host cell DNA per dose.

35. A method of preparing a synthetic expression construct which encodes a viral segment from an influenza virus, comprising:
a) providing the sequence of at least part of the coding region of the HA or NA segment from an influenza virus;
b) identifying the HA and/or NA subtype of the influenza virus from which the coding region is derived;
c) providing a UTR sequence from an influenza virus with the same HA or NA subtype as the subtype identified in step (b); and
d) preparing a synthetic expression construct which encodes a viral segment comprising the coding sequence and the UTR.

36. The method of claim 1, wherein the cell is a mammalian cell or an avian cell.

37. The method of claim 36, wherein the cell is an MDCK, Vero or PerC6 cell.

38. The method of claim 37, wherein the cell is of the cell line MDCK 33016 (DSM ACC2219).

39. The method of claim 36, wherein the cell grows in suspension.

40. The method of claim 1, wherein the cell grows adherently.

41. A library comprising two or more influenza backbones.
